SEQUENCE LISTING (44 sequences)

SEQ ID NO: 1 - protein, 121 aa, Artificial Sequence
  Synthetic GA1 - Heavy chain variable domain region
QVQLQQSGPGLVRPSQTLSLSCDISGDSVSSNSATWNWIRQSPSRGLEWLGRTFYRSKWYHDYALSVKSRITINPDTSKNQFSLQLNSVSPGDTAVYFCVREDIDGRLDYWGQGTLVTVSS

SEQ ID NO: 2 - protein, 111 aa, Artificial Sequence
  Synthetic GA1 - Light chain variable domain region
LPVLTQPASVSGSPGQSITISCTGTSSDVGGYNYVSWYQQHPGKAPKLMIYEVSKRPSGVPDRFSGSKSGNTASLTVSGLQAEDEADYYCSAWDDSLNADVFGGGTKLTVL

SEQ ID NO: 3 - protein, 119 aa, Artificial Sequence
  Synthetic GA2 - Heavy chain variable domain region
QVQLVQSGSELKKPGASVKISCKASGYIFSDNGVNWVRQAPGQGLEWMGWINTKIGNPTYAQGFTGRFVFSLDTSVSTTYLQISSLQAGDTAVYYCAREHDYYYGMDVWGQGTTVTVSS

SEQ ID NO: 4 - protein, 110 aa, Artificial Sequence
  Synthetic GA2 - Light chain variable domain region
QPVLTQPPSASGTPGQRVTISCSGSSSNIGTNTVNWYQQVPGTAPKLLIHGNDQRPSGVPDRFSGSKSDTSASLAITGLQSDDDADYYCSAWDDSLNADVFGGGTKLTVL

SEQ ID NO: 5 - protein, 122 aa, Artificial Sequence
  Synthetic GB1 - Heavy chain variable domain region
MAEVQLLESGAEVKKPGASVKVSCKASGYTFTSHYMHWVRQAPGQGLEWMGVINPSGGSTSYAQKFQGRVTMTRDTSTSTVYMDLSSLRSEDTAVYYCARRSEAYYHGMDVWGQGTTVTVSS

SEQ ID NO: 6 - protein, 110 aa, Artificial Sequence
  Synthetic GB1 - Light chain variable domain region
QAVLTQPPSASATPGQRVTISCSGSDSNIGTNYVYWYQQFPGTAPQPLIYRDNQRPSGVPDRFSGSKSGTSASLAISGLRSEDEATYFCSTWDDSLNGWVFGGGTKLTVL

SEQ ID NO: 7 - protein, 119 aa, Artificial Sequence
  Synthetic GB6 - Heavy chain variable domain region
EVQLVQSGSELKKPGASVKISCKASGYIFSDNGVNWVRQAPGQGLEWMGWINTKIGNPTYAQGFTGRFVFSLDTSISTTYLQISSLQAGDTAVYYCAREHDYYYGMDVWGQGTTVTVSS

SEQ ID NO: 8 - protein, 111 aa, Artificial Sequence
  Synthetic GB6 - Light chain variable domain region
QPVLTQPRSVSGSPGQSITTSCTGTSSDVGGYNYVSWYQQHPGKAPKLMIYEVSNRPSGVSNRFSGSKSGNTASLTISGLQAEDEADYYCSSYTSSSTLEVFGTGTKVTVL

SEQ ID NO: 9 - protein, 131 aa, Artificial Sequence
  Synthetic GH1 - Heavy chain variable domain region
QVQLVESGGGLVQPGGSLRLSCEATGFTFSRYWMHWVRQAPGKGLEWVSSISSSSSYIYYADSVKGRFTISRDNAKNSLYLQMNSLRAEDTAVYYCARDTLEYYGSGILENAMGYYGMDVWGQGTTVTVSS

SEQ ID NO: 10 - protein, 111 aa, Artificial Sequence
  Synthetic GH1 - Light chain variable domain region
SYELMQPPSVSGAPGQRVTISCTGSSSNIGAAYDVHWYQQLPGKAPKLVMFANSNRPSGVPDRFSGSKSGTSASLAITGLQAEDEADYYCQSYDISLRAYVFGTGTKLTVL

SEQ ID NO: 11 - protein, 119 aa, Artificial Sequence
  Synthetic A2 -Heavy chain variable domain region
EVQLVESGGGLVRPGGSLRLACAASGFSFSDYYMTWIRQAPGRGLEWIAYISDSGQTVHYADSVKGRFTISRDNTKNSLFLQVNTLRAEDTAVYYCAREDLLGYYLQSWGQGTLVTVSS

SEQ ID NO: 12 - protein, 110 aa, Artificial Sequence
  Synthetic A2 -Light chain variable domain region
SYELMQPPSASGTPGQRVTISCSGSSSNIGTNTVNWYQHLPGTAPKLLIYSNNQRPSGVPDRFSGSKSGTSASLAISGLQSEDEADYYCATWDDSPNGWVFGGGTKLTVL

SEQ ID NO: 13 - protein, 118 aa, Artificial Sequence
  Synthetic C7 -Heavy chain variable domain region
EVQLVESGGGVVQPGRSLRLSCAASGFTFSSYWMSWVRQAPGKGLEWVANIKQDGSEKYYVDSVKGRFTISRDNAKNSLYLQMNSLRAEDTAVYYCAREGEHDAFDIWGQGTMVTVSS

SEQ ID NO: 14 - protein, 109 aa, Artificial Sequence
  Synthetic C7 -Light chain variable domain region
QAVLTQPPSVSAAPGQRVTISCSGSNSNIADTYVSWYQQLPGTAPRLLIYDNDQRPSGIPDRFSGSKSGTSATLGITGLQTGDEADYYCGTWDSSLSGVFGTGTKVTVL

SEQ ID NO: 15 - protein, 119 aa, Artificial Sequence; identical to SEQ ID NO: 3
  Synthetic H7 -Heavy chain variable domain region

SEQ ID NO: 16 - protein, 109 aa, Artificial Sequence
  Synthetic H7 -Light chain variable domain region
QSVLTQPASVSGSPGQSVTISCTGSSSDVGAYNFVSWYRQYPGKAPKLLIYEVNKRPSDVPDRFSGSKFGNTASLTVSGLQADDEADYYCSSYAGSTDVFGTGTKVTVL

SEQ ID NO: 17 - protein, 119 aa, Artificial Sequence; identical to SEQ ID NO: 3
  Synthetic SH-A4 - Heavy chain variable domain region

SEQ ID NO: 18 - protein, 110 aa, Artificial Sequence
  Synthetic SH-A4 - Light chain variable domain region
LPVLTQPPSVSGTPGQRVTISCSGSSSNIGSNTVNWYQQLPGTAPKLLIYTNNQRPSGVPDRFSGSKSGTSASLAISGLQSEDEADYYCAAWDESLNGDVFGTGTKVTVL

SEQ ID NO: 19 - protein, 120 aa, Artificial Sequence
  Synthetic SH-A9 - Heavy chain variable domain region
QVQLVQSGAEVKKPGASVKVSCKASGYTFTRYTMHWVRQAPGQRLEWMGWINAGNGNTKYSQKFQGRVTITRDTSASTAYMELSSLRSEDTAVYYCAKVSAGTESWFDPWGQGTLVTVSS

SEQ ID NO: 20 - protein, 107 aa, Artificial Sequence
  Synthetic SH-A9 - Light chain variable domain region
AIRMTQSPSFLSASVGDRVTITCRTSQNIYNYLNWYQQKPGKAPELLIFVASSLQSGVPSRFSGSGSGTDFTLTISSLQPEDFATYFCLQDHSYPYTFGQGTKVEIK

SEQ ID NO: 21 - protein, 117 aa, Artificial Sequence
  Synthetic RG1B3 - Heavy chain variable domain region
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGLYGDEDYWGQGTLVTVSS

SEQ ID NO: 22 - protein, 110 aa, Artificial Sequence
  Synthetic RG1B3 - Light chain variable domain region
LPVLTQPPSVSEVPGQRVTISCSGGISNIGSNAVNWYQHFPGKAPKLLIYYNDLLPSGVSDRFSASKSGTSASLAISGLRSEDEADYYCAAWDDNLSAYVFATGTKVTVL

SEQ ID NO: 23 - protein, 119 aa, Artificial Sequence
  Synthetic RG1H10 -Heavy chain variable domain region
QVQLVQSGSELKKPGASVKISCKASGYIFSDNGVNWVRQAPGQGLEWMGWINTKIGNPTYAQGFTGRFVFSLDTSISTTYLQISSLQAGDTAVYYCAREHDYYYGMDVWGQGTTVTVSS

SEQ ID NO: 24 - protein, 111 aa, Artificial Sequence
  Synthetic RG1H10, RG1H10-H2A-22-1S, RG1H10-H2A-27-2S, RG1H10-3C, RG1H10-16C, RG1H10-17C, RG1H10-19C, RG1H10-21C, RG1H10-23C2 - Light chain variable domain region
QSALTQPPSASGSPGQSVTISCTGTSSDVGGYNYVSWYQHHPGKAPKLMIYEVSKRPSGVPDRFSGSKSAITASLTISGLLTEDEADYYCSAWDDSLNADVFGGGTKVTVL

SEQ ID NO: 25 - protein, 125 aa, Artificial Sequence
  Synthetic RG1H11 -Heavy chain variable domain region
QMQLVQSGAEVKKPGESLKISCKGSGYSFTSYWIGWVRQMPGKGLEWMGIIYPGDSDTRYSPSFQGQVTISADKSISTAYLQWSSLKASDTAMYYCASGVTRKRYSSSWPPFDYWGQGTLVTVSS

SEQ ID NO: 26 - protein, 107 aa, Artificial Sequence
  Synthetic RG1H11 -Light chain variable domain region
DIQMTQSPSSLSASVGDRVTITCRASQSISTYLNWYQQKPGKAPKVLITDASSLETGVPSRFSGSGSGTDFTFTISSLQPEDTATYFCQQYDDLPPTFGQGTKLEIK

SEQ ID NO: 27 - protein, 120 aa, Artificial Sequence
  Synthetic RG2H7 - Heavy chain variable domain region
QVQLQQWGAGLLKSSETLSLSCAVYGGTFRDDHWSWIRQPPGKGLEWIGESHHTGRTIYNPSLRSRVTMSIDTSKNEFSLILRSVTAADTATYFCARGNNYVWGNQEDFWGQGTLVTVSS

SEQ ID NO: 28 - protein, 110 aa, Artificial Sequence
  Synthetic RG2H7 - Light chain variable domain region
QAGLTQPRSVSGSPGQSVTISCTGTSSDVGGYNYVSWYQQHPGKAPKLMIYDVTKRPSGVSNRFSGSKSGNTASLTISGLQAEDEADYYCSSYTSSSTYVFGTGTKVTVL

SEQ ID NO: 29 - protein, 121 aa, Artificial Sequence; identical to SEQ ID NO: 1
  Synthetic RG2H10 -Heavy chain variable domain region

SEQ ID NO: 30 - protein, 111 aa, Artificial Sequence
  Synthetic RG2H10 -Light chain variable domain region
QPVLTQPASVSGSPGQSITISCTGTSSDVGGYNYVSWYQQHPGKAPKLMIYDVSNRPSGVSNRFSGSKSGNTASLAITGLQSDDDADYYCSAWDDSLNADVFGGGTKLTVL

SEQ ID NO: 31 - protein, 123 aa, Artificial Sequence
  Synthetic RG3E12 -Heavy chain variable domain region
EVQLVESGGALVQPGGSLRLSCAVSGFTFSDHYMDWVRQAPGKGLEWVARSRNKGNSYTTEYAASVRGRFTISRDDSKNSLYLQMNSLKTEDTAVYYCVRVGVVPALDGMDVWGQGTTVTVSS

SEQ ID NO: 32 - protein, 110 aa, Artificial Sequence
  Synthetic RG3E12 -Light chain variable domain region
QAGLTQPPSVSKGLRQTATLTCTGNSNNIGDQGAAWLQQHQGHPPRLLSYRNNNRPSGISERLSASRSGNIASLTITGLQPEDEADYYCSAWDSSLSVWVFGGGTKLTVL

SEQ ID NO: 33 - protein, 131 aa, Artificial Sequence
  Synthetic RG4A6 - Heavy chain variable domain region
EVQLVESGGGLVQPGGSLRLSCAASGFTFSSYWMHWVRQAPGKGLEWVSSISSSSSYIYYADSVKGRFTISRDNAKNSLYLQMNSLRAEDTAVYYCARDTLEYYGSGILENAMGYYGMDVWGQGTTVTVSS

SEQ ID NO: 34 - protein, 110 aa, Artificial Sequence
  Synthetic RG4A6 - Light chain variable domain region
QAVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGYVFGTGTKLTVL

SEQ ID NO: 35 - protein, 123 aa, Artificial Sequence
  Synthetic RG5D9 - Heavy chain variable domain region
EVQLLESGGGVVQTGRSLRLSCSDSGSTFRSQAMHWVRQTPGKGLEWLAVTSHDGSKTYYADSVKGRFTISRDNSKNTLYLQMNSLRGEDTAVYYCARGGRGYTYDHSFFDYWGQGTLVTVSS

SEQ ID NO: 36 - protein, 107 aa, Artificial Sequence
  Synthetic RG5D9 - Light chain variable domain region
AIRMTQSPSTLSASVGDRVTITCRASENIRNLLAWYQQKPGKAPELLIHGASTLGTGVPSRFSGGGSGTEFTLTISSLQPDDFATYYCQQYESYFNTFGQGTKVEIK

SEQ ID NO: 37 - protein, 119 aa, Artificial Sequence
  Synthetic RG1H10-H2A-22-1S -Heavy chain variable domain region
QVQLVQSGSELKKPGASVKISCKASGYIFSDNGVNWVRQAPGQGLEWMGWINTKDGNPTYAQGFTGRFVFSLDTSISTTYLQISSLQAGDTAVYYCAREHDYYYGMDVWGQGTTVTVSS

SEQ ID NO: 38 - protein, 119 aa, Artificial Sequence
  Synthetic RG1H10-H2A-27-2S -Heavy chain variable domain region
QVQLVQSGSELKKPGASVKISCKASGYIFSDNGVNWVRQAPGQGLEWMGWINTKTGNPTYAQGFTGRFVFSLDTSISTTYLQISSLQAGDTAVYYCAREHDYYYGMDVWGQGTTVTVSS

SEQ ID NO: 39 - protein, 119 aa, Artificial Sequence
  Synthetic RG1H10-3C - Heavy chain variable domain region
QVQLVQSGSELKKPGASVKISCKASGYTFSDNGVNWVRQAPGQGLEWMGWINTKDGNPTYAQGFTGRFVFSLDTSISTTYLQISSLQAGDTAVYYCAREHDYYYGMDVWGQGTTVTVSS

SEQ ID NO: 40 - protein, 119 aa, Artificial Sequence
  Synthetic RG1H10-16C -Heavy chain variable domain region
QVQLVQSGSELKKPGASVKISCKASGYTFSDNGVNWVRQAPGQGLEWMGWINTKTGNPTYAQGFTGRFVFSLDTSISTTYLQISSLQAGDTAVYYCAREHDYYYGMDVWGQGTTVTVSS

SEQ ID NO: 41 - protein, 119 aa, Artificial Sequence
  Synthetic RG1H10-17C -Heavy chain variable domain region
QVQLVQSGSELKKPGASVKISCKASGYKFSDNGVNWVRQAPGQGLEWMGWINTKSGNPTYAQGFTGRFVFSLDTSISTTYLQISSLQAGDTAVYYCAREHDYYYGMDVWGQGTTVTVSS

SEQ ID NO: 42 - protein, 119 aa, Artificial Sequence
  Synthetic RG1H10-19C -Heavy chain variable domain region
QVQLVQSGSELKKPGASVKISCKASGYKFSDNGVNWVRQAPGQGLEWMGWINTKTGNPTYAQGFTGRFVFSLDTSISTTYLQISSLQAGDTAVYYCAREHDYYYGMDVWGQGTTVTVSS

SEQ ID NO: 43 - protein, 119 aa, Artificial Sequence
  Synthetic RG1H10-21C -Heavy chain variable domain region
QVQLVQSGSELKKPGASVKISCKASGYTFSDNGVNWVRQAPGQGLEWMGWINTKSGNPTYAQGFTGRFVFSLDTSISTTYLQISSLQAGDTAVYYCAREHDYYYGMDVWGQGTTVTVSS

SEQ ID NO: 44 - protein, 119 aa, Artificial Sequence
  Synthetic RG1H10-23C2 - Heavy chain variable domain region
QVQLVQSGSELKKPGASVKISCKASGYKFSDNGVNWVRQAPGQGLEWMGWINTKDGNPTYAQGFTGRFVFSLDTSISTTYLQISSLQAGDTAVYYCAREHDYYYGMDVWGQGTTVTVSS

(19) United States
(12) Patent Application Publication
(10) Pub. No.: US 2024/0279339 A1
(43) Pub. Date: Aug. 22, 2024

(54) METHOD OF TREATING CANCER BY ADMINISTRATION OF AN ANTI-PD-1 OR ANTI-PD-L1 THERAPEUTIC AGENT VIA A LYMPHATIC MICRONEEDLE DELIVERY DEVICE

(71) Applicant: VIVASOR, INC., San Diego, CA (US)

(72) Inventors: Russell Frederick Ross, San Diego, CA (US); David Francis, San Diego, CA (US)

(21) Appl. No.: 18/568,260

(22) PCT Filed: Jun. 8, 2022
(86) PCT No.: PCT/US2022/032747

§ 371 Date: Dec. 7, 2023

Related U.S. Application Data
(60) Provisional application No. 63/208,804, filed on Jun. 9, 2021.

(51) Int. Cl. C07K 16/28; A61K 9/00; A61K 39/00; A61P 35/00

(52) U.S. Cl. CPC C07K 16/2827; A61K 9/0021; A61P 35/00; C07K 16/2818; A61K 2039/505; A61K 2039/54

(57) ABSTRACT

Methods, devices and systems for delivery of an anti-PD-1 or an anti-PD-L1 therapeutic agent to the lymphatic system for treating cancer in a patient are described.

### CROSS REFERENCE TO RELATED APPLICATIONS

This application claims priority to U.S. provisional patent application No. 63/208,804, filed on Jun. 9, 2021, the content and disclosure of which is incorporated by reference in its entirety for all purposes.

### TECHNICAL FIELD

The present disclosure relates to methods, devices and systems for administration of immune checkpoint anti-PD-1 or anti-PD-L1 therapeutic agents for treatment of cancer in patients.

### BACKGROUND

Checkpoint inhibitors are a form of cancer therapy that directly affect the functioning of the immune system of the patient. Immune system checkpoints can be either stimulatory or inhibitory, and some cancers are known to affect these checkpoints to prevent the immune system from attacking them. As such, checkpoint inhibitors can block these inhibitory checkpoints thereby restoring proper immune system function. Examples of checkpoints include, but are not limited to, CTLA-4, PD-1, and PD-L1. Some checkpoint inhibitors that are currently approved by the FDA include, but are not limited to, ipilimumab (CTLA-4 inhibitor; sold under the tradename of Yervoy®, Bristol-Myers Squibb Company, Delaware), nivolumab (PD-1 inhibitor; sold under the tradename of Opdivo®, Bristol-Myers Squibb), pembrolizumab (PD-1 inhibitor; sold under the tradename of Keytruda®, Merck Sharp & Dohme, New Jersey), and atezolizumab (PD-L1 inhibitor; sold under the tradename of Tecentriq®, Genentech, Inc., Delaware). As used herein, the term checkpoint inhibitor encompasses therapeutic agents that are used to modulate the activity of an immune system checkpoint.

Side effects of targeting molecules of the immune system include immune-related Adverse Events (irAEs). Accordingly, there is need to develop dosing regimens or methods that maintain a therapeutically effective dose of the therapeutic agent in a patient while reducing the overall patient exposure to the therapeutic agent, thereby reducing or minimizing irAEs in such patient.

### SUMMARY

The present disclosure provides a method of treating cancer in a patient. The method includes placing a device comprising a plurality of microneedles on the skin of the patient proximate to a first position under the skin of the patient, wherein the first position is proximate to lymph vessels and/or lymph capillaries in the patient's lymphatic system, and wherein the microneedles have a surface comprising nanotopography; inserting the plurality of microneedles into the patient to a depth whereby at least the epidermis is penetrated and an end of at least one of the microneedles is proximate to the first position; and administering via the plurality of microneedles to the first position an effective amount of an anti-PD-1 therapeutic agent or an effective amount of an anti-PD-L1 therapeutic agent to treat cancer in a patient. The present disclosure also provides devices and/or systems configured for administering an anti-PD-1 or anti-PD-L1 therapeutic agent, wherein the device and/or system comprises an amount of an anti-PD-1 therapeutic agent or anti-PD-L1 therapeutic agent effective for treating cancer in a patient.

The present disclosure also provides a method of preventing or reducing cancer metastasis in a patient. The method includes locating at least one lymph node in the patient that intervenes in the lymphatic system between a solid cancer tumor and a draining duct; placing a device comprising a plurality of microneedles on the skin of the patient proximate to a first position under the skin of the patient located between the intervening lymph node and the solid cancer tumor, wherein the first position is proximate to lymph vessels and/or lymph capillaries in the patient's lymphatic system, and wherein the microneedles have a surface comprising nanotopography; inserting the plurality of microneedles into the patient to a depth whereby at least the epidermis is penetrated and an end of at least one of the microneedles is proximate to the first position; and administering via the plurality of microneedles to the first position a therapeutically effective amount of an anti-PD-1 therapeutic agent or an anti-PD-L1 therapeutic agent that is effective for preventing or reducing cancer metastasis in the patient. The present disclosure also provides devices and/or systems configured for administering an anti-PD-1 or anti-PD-L1 therapeutic agent, wherein the device and/or system comprises an amount of an anti-PD-1 therapeutic agent or anti-PD-L1 therapeutic agent effective for preventing or reducing cancer metastasis in a patient.

The present disclosure also provides a method of preventing or reducing cancer metastasis in a patient, the method including locating a solid cancer tumor in the patient; locating at least one lymph node in the patient that intervenes in the lymphatic system between the solid cancer tumor and a draining duct; placing a device that comprises a plurality of microneedles on the skin of the patient at a first location on the skin of the patient that is proximate to lymph capillaries and/or lymph vessels that flow into the intervening lymph node, wherein the microneedles have a surface comprising nanotopography; inserting the plurality of microneedles into the patient to a depth whereby at least the epidermis is penetrated; and administering via the plurality of microneedles to the lymph capillaries and/or lymph vessels that flow into the intervening lymph node a therapeutically effective amount of an anti-PD-1 therapeutic agent or an anti-PD-L1 therapeutic agent that is effective in preventing or reducing cancer metastasis in the patient. The present disclosure also provides devices and/or systems configured for administering an anti-PD-1 or anti-PD-L1 therapeutic agent, wherein the device and/or system comprises an amount of an anti-PD-1 therapeutic agent or anti-PD-L1 therapeutic agent effective for preventing or reducing cancer metastasis in a patient.

According to various further embodiments of the methods, devices and/or systems, which may all be combined with one another unless clearly mutually exclusive:
i) the cancer may comprise a tumor.
ii) the lymph node may be a tumor draining lymph node.
iii) the cancer may be a cancer susceptible to treatment with an anti-PD-1 therapeutic agent or anti-PD-L1 therapeutic agent.
iv) serum bioavailability of the anti-PD-1 therapeutic agent or the anti-PD-L1 therapeutic agent may be up to 10%, 15% 20%, 25%, 30%, 35%, 40%, 45%, 50%, 55%, 60%, 65%, or 70%.
v) a serum Tmax of the anti-PD-1 therapeutic agent or the anti-PD-L1 therapeutic agent may be from 10 to 100 hours.
vi) a serum Cmax of the anti-PD-1 therapeutic agent or the anti-PD-L1 therapeutic agent may be decreased up to 1.5-fold, 2-fold, 2.5-fold, 3-fold, 3.5-fold, or 4-fold, as compared to a serum Cmax following an equivalent amount of the anti-PD-1 therapeutic agent or the anti-PD-L1 therapeutic agent administered by an intravenous delivery route.
vii) a serum AUC₀₋ₜ of the anti-PD-1 therapeutic agent or the anti-PD-L1 therapeutic agent may be decreased up to 1.5-fold, 2-fold, 2.5-fold, 3-fold, 3.5-fold, or 4-fold as compared to a serum AUC₀₋ₜ following an equivalent amount of the anti-PD-1 therapeutic agent or the anti-PD-L1 therapeutic agent administered by an intravenous delivery route.
viii) delivery of the anti-PD-1 therapeutic agent or the anti-PD-L1 therapeutic agent to one or more lymph nodes may be increased up to 1.5-fold, 2-fold, 2.5-fold, 3-fold, 3.5-fold, or 4-fold as compared to delivery to one or more lymph nodes of an equivalent amount of the anti-PD-1 therapeutic agent or the anti-PD-L1 therapeutic agent administered by an intravenous delivery route.
ix) levels of the anti-PD-1 therapeutic agent or anti-PD-L1 therapeutic agent in one or more systemic organs may be decreased 10-75% over a period of time as compared to levels in one or more systemic organs following an equivalent amount of the anti-PD-1 therapeutic agent or the anti-PD-L1 therapeutic agent administered by an intravenous delivery route over a same period of time.
x) the organ may be a liver or a kidney.
xi) the period of time may be up to 12 hours, 24 hours, 36 hours, 48 hours, 60 hours, or 72 hours.
xii) the anti-PD-1 therapeutic agent or the anti-PD-L1 therapeutic agent may be at least 90%, 95%, 99%, or 99.9% cleared from the patient's serum by 28 days after administration.
xiii) the administering may result in an undetectable primary tumor and/or an undetectable secondary tumor.
xiv) an incidence or probability of an undetectable primary tumor and/or an undetectable secondary tumor may be at least 30%, 40%, 50%, 60%, 70%, 80%, 90%, 95%, or 99%.
xv) exposure of the anti-PD-1 therapeutic agent or the anti-PD-L1 therapeutic agent to T-cells in the patient's lymphatic system may be increased up to 1.5-fold, 2-fold, or 2.5-fold, as compared to exposure to T-cells in the patient's lymphatic system following an equivalent amount of the anti-PD-1 therapeutic agent or the anti-PD-L1 therapeutic agent administered by an intravenous delivery route.
xvi) exposure of the anti-PD-1 therapeutic agent or the anti-PD-L1 therapeutic agent to one or more solid cancer tumors in the patient's lymphatic system may be increased up to 1.5-fold, 2-fold, or 2.5-fold, as compared to exposure to one or more solid cancer tumors in the patient's lymphatic system following an equivalent amount of the anti-PD-1 therapeutic agent or the anti-PD-L1 therapeutic agent administered by an intravenous delivery route.
vii) tumor-infiltrating lymphocytes may be increased up to 1.5-fold, 2-fold, or 2.5-fold as compared to tumor-infiltrating lymphocytes following an equivalent amount of the anti-PD-1 therapeutic agent or the anti-PD-L1 therapeutic agent administered by an intravenous delivery route.
xviii) incidence or severity of one or more immune-related Adverse Events may be reduced as compared to one or more immune-related Adverse Events following an equivalent amount of the anti-PD-1 therapeutic agent or the anti-PD-L1 therapeutic agent administered by an intravenous delivery route.
xix) tumor growth inhibition may be increased up to 10-fold, 20-fold, 30-fold, 40-fold, or 50-fold compared to tumor growth inhibition following an equivalent amount of the anti-PD-1 therapeutic agent or the anti-PD-L1 therapeutic agent administered by an intravenous delivery route.
xx) tumor growth inhibition may be equal to or better than tumor growth inhibition following up to 1.5-fold, 2-fold, 2.5-fold, 3-fold, 3.5-fold, 4-fold, 4.5-fold, or 5-fold greater amount of the anti-PD-1 therapeutic agent or the anti-PD-L1 therapeutic agent administered by an intravenous delivery route.

### BRIEF DESCRIPTION OF THE DRAWINGS

For a more complete understanding of the present disclosure and the associated features and advantages, reference is now made to the following description, taken in conjunction with the accompanying drawings, which are not to scale, and in which:

FIG. 1 is a schematic of (i) an example SOFUSA® (Sorrento Therapeutics, Inc. Corporation Delaware) Nanotopographical Device for infusing anti-PD-1 or anti-PD-L1 into the sub-epidermal space, which includes a (ii) microfluidic fluid block with a microfluidic distributor (green) and silicon microneedle array (gray). As an exemplary embodiment, each microneedle is 350 μm long, 110 μm wide with a 30 μm hole located off center through which the therapeutic agent flows out. (iii) SOFUSA® infuses therapeutic agent into the sub-epidermal space where the initial lymphatics provide uptake, whereas deeper subcutaneous injections deposit drug below the initial lymphatics, reducing uptake.

FIG. 2 is a schematic of an example SOFUSA® Nanotopographical Device, showing (i) the Microfluidic Fluid Block with a perforated attachment adhesive (tan), microfluidic distributor (green), perforated attachment adhesive (yellow), and silicon microneedle array (gray). Each microneedle is 350 μm long, 110 μm wide with a 30 μm hole located off center through which the drug flows out. Also shown is (ii) Scanning Electron Microscopy (SEM) image of nanotopographic film heat formed over the silicon microneedles (scale bar represents 300 m) and (iii) SEM of individual microneedle, and (iv) SEM image of the nanostructures on each microneedle (scale bar=3 μm).

FIG. 3 is (i) a schematic showing example placement of SOFUSA® on dorsal back of mice and Near-Infrared Fluorescence (NIRF) (ii) dorsal and (iii) lateral images of SOFUSA® delivery of indocyanine green (ICG) to brachial lymph nodes (LN).

FIG. 4A and FIG. 4B include a set of example images showing in FIG. 4A, left: Applied SOFUSA® device infusing ICG and FIG. 4A, right: NIRF imaging of lymphatic vessels propelling ICG-laden lymph during infusion (right) and intradermal (i.d.) injection (left) of ICG in the medial ankle and lateral calf with arrows showing i.d. injection and SOFUSA® infusion placement. FIG. 4B. is example near-infrared fluorescence images of SOFUSA® delivery of ICG into the axilla and inguinal LNs of healthy volunteers.

FIG. 5A is an example graph reporting the average±SE ratio of lymphatic contractile pumping in lymphatic vessels draining SOFUSA® infusion sites to that in vessels draining from contralateral i.d. injections sites as a function of infusion flowrate for administration sites in the arm, ankle, and calf. The ratios reported in FIG. 5A were obtained by dividing the SOFUSA® infusion data by the intradermal infusion data, for each body region, at each time point indicated.

FIG. 5B is example photographs of tissue sites after removal of SOFUSA® device that infused ICG at rates of less than 1 mL/h (left) and at 1 mL/h (right) with the latter showing “pooling” of ICG in the epidermis.

FIG. 6 is a schematic of an example site of PD-1 or PD-L1 as drug targets for locally advanced metastatic cancer. Inset (A) shows in tumor-draining LNs, T-cell activation to antigen-presenting cells (APC) presenting tumor antigen (Ag) can be inhibited by PD-L1 and blocking inhibition by administration of anti-PD-L1 or anti-PD-1 could result in T-cell activation against tumor Ag. Inset (B) shows in regional or distant LNs where tumor Ag may not be presented, anti-PD-1 or anti-PD-L1 blocking of inhibition against T-cell activation to APC cells presenting self Ag response can create immune-related Adverse Events (irAEs). Inset (C) shows a tumor cell displaying PD-L1 on its cell surface and anti-PD-1 or anti-PD-L1 blocking PD-1:PD-L1 suppression of T-cell response against the tumor cell. Modified from FIG. 1 of Sunkuk Kwon, Fred Christian Velasquez, John C. Rasmussen, Matthew R. Greives, Kelly D. Turner, John R. Morrow, Wen-Jen Hwu, Russell F. Ross, Songlin Zhang, and Eva M. Sevick-Muraca (2019), Nanotopography-based lymphatic delivery for improved anti-tumor responses to checkpoint blockade immunotherapy, Theranostics, 9(26): 8332-8343, doi:10.7150/thno.35280.

FIG. 7 is a graph reporting example PK curves for the SOFUSA® DoseConnect™ and intravenous administrations of anti-PD-L1 mAb in C57/BL6 mice.

FIG. 8 is a graph reporting example PK curves for the SOFUSA® DoseConnect™ and intravenous administrations of anti-PD-L1 mAb labelled with ⁸⁹Zr radioisotope in C57/BL6 mice.

FIG. 9 is a graph reporting example anti-PD-L1 mAb lymph node concentrations for SOFUSA® DoseConnect™ and intravenous delivery.

FIG. 10A and FIG. 10B is a pair of graphs reporting example biodistribution for ⁸⁹Zr-anti-PD-L1 mAb in healthy C57/BL6 mice at 1 hour after beginning SOFUSA® DoseConnect™ (FIG. 10A) and 1 hour after intravenous administration (FIG. 10B)

FIG. 11A and FIG. 11B is a pair of graphs reporting example biodistribution for ⁸⁹Zr-anti-PD-L1 mAb in healthy C57/BL6 mice at 24 hours after beginning SOFUSA® DoseConnect™ (FIG. 11A) and 24 hours after intravenous administration (FIG. 11B)

FIG. 12A and FIG. 12B is a pair of graphs reporting example biodistribution for ⁸⁹Zr-anti-PD-L1 mAb in healthy C57/BL6 mice at 72 hours after beginning SOFUSA® DoseConnect™ (FIG. 12A) and 72 hours after intravenous administration (FIG. 12B)

FIG. 13 is a Table of information of the example treatment groups and dosing schedules in a multi-phase exploratory toxicity and toxicokinetics study of anti-PD1 monoclonal antibody STI-A1110 (Sorrento Pharmaceuticals) following lymphatic delivery (SOFUSA® DoseConnect™) in cynomolgus monkeys.

FIG. 14 is a Table of information of the example clinical pathology assay information in the multi-phase exploratory toxicity and toxicokinetics study of anti-PD1 monoclonal antibody STI-A1110 (Sorrento Pharmaceuticals).

FIG. 15A is a graph reporting example pharmacokinetic (PK) enzyme-linked immunosorbent assay (ELISA) results of serum concentrations of anti-PD1 monoclonal antibody STI-A1110 (Sorrento Pharmaceuticals) following intravenous dosing in monkey 4501 and lymphatic delivery using SOFUSA® DoseConnect™ in monkey 6501.

FIG. 15B is another graph reporting example pharmacokinetic (PK) enzyme-linked immunosorbent assay (ELISA) results of serum concentrations of anti-PD1 monoclonal antibody STI-A1110 (Sorrento Pharmaceuticals) following intravenous dosing in monkeys 4501 and 4001 and lymphatic delivery using SOFUSA® DoseConnect™ in monkeys 6501 and 6001.

FIG. 15C is a graph reporting example comparison of platelet levels in monkeys after 40 mg/kg dosing of anti-PD1 monoclonal antibody STI-A1110 (Sorrento Pharmaceuticals) intravenously or using SOFUSA® DoseConnect™.

FIG. 16 is a Table of example dosing of pembrolizumab in CTCL patients using the SOFUSA® DoseConnect™.

FIG. 17A is a graph reporting example average serum concentration of anti-PD1 monoclonal antibody STI-2949 (Sorrento Pharmaceuticals; also referred to herein as STI-A1110) in Sprague Dawley rats following a single intravenous injection of STI-2949 on day 0, or following a single administration of STI-2949 on day 0 using SOFUSA® DoseConnect™.

FIG. 17B is a graph reporting example serum concentration of anti-PD1 monoclonal antibody STI-2949 (Sorrento Pharmaceuticals) in individual Sprague Dawley rats following a single intravenous injection of STI-2949 on day 0, or following a single administration of STI-2949 on day 0 using SOFUSA® DoseConnect™ FIG. 18A is example serum radiance images and a graph reporting example average radiance signal in blood taken from mice following administration of a fluorescently labeled anti-PD1 monoclonal antibody STI-2949 (Sorrento Pharmaceuticals) intaperitoneally (i.p.) or using SOFUSA® DoseConnect™.

FIG. 18B is example images of radiance in draining- or non-draining lymph nodes and a graph reporting example average radiance signal in draining- or non-draining lymph nodes taken from mice following administration of a fluorescently labeled anti-PD1 monoclonal antibody STI-2949 (Sorrento Pharmaceuticals) i.p. or using SOFUSA® DoseConnect™ FIG. 19A is a schematic of an example timeline of a study investigating anti-PD1 (BB9 clone, Sorrento Pharmaceuticals) administered i.p. or using SOFUSA® DoseConnect™ on tumor growth in an MC38 colon carcinoma mouse model.

FIG. 19B is a graph reporting example percentage tumor growth inhibition in an MC38 colon carcinoma mouse model following anti-PD1 monoclonal antibody (BB9 clone, Sorrento Pharmaceuticals) administered i.p. or using SOFUSA® DoseConnect™, at doses of 10, 5, or 1 mg/kg.

### DETAILED DESCRIPTION

The present disclosure generally relates to methods of delivery to the lymphatic system, for example using a nanotopography-based microneedle array, for improved checkpoint blockade immunotherapy using an anti-PD-1 or anti-PD-L1 therapeutic agent. The present disclosure also relates to devices and systems described herein configured for lymphatic delivery of an effective amount of an anti-PD-1 or anti-PD-L1 therapeutic agent. As described herein, advantages of the methods disclosed herein using the lymphatic administration of these immune checkpoint blockade inhibitors include, inter alia, reducing systemic exposure to the therapeutic agent, and maximizing delivery of the therapeutic agent to tumor-draining lymph nodes (TDLNs) where tumor antigens (Ags) are present, as well as maximizing delivery of the therapeutic agent to tumors.

Checkpoint inhibitor therapy is a form of cancer immunotherapy. The therapy targets immune checkpoints, key regulators of the immune system that when stimulated can dampen the immune response to an immunologic stimulus. Some cancers can protect themselves from attack by stimulating immune checkpoint targets. Checkpoint inhibitor therapy can block inhibitory checkpoints, restoring immune system function.

The terms “anti-PD-1 therapeutic agent” and “anti-PD-L1 therapeutic agent” as used herein refer to any molecule that is capable of inhibiting an immune checkpoint function of PD-1 and/or PD-L1, respectively. The anti-PD-1 and anti-PD-L1 therapeutic agents of the present disclosure include, without limitation, molecules capable of binding to and inhibiting an immune checkpoint function of PD-1 and/or PD-L1, respectively, such as antibodies, including but not limited to monoclonal antibodies, fully human antibodies, humanized antibodies, chimeric antibodies, IgG1 antibodies, IgG2 antibodies, IgG3 antibodies, IgG4 antibodies, antigen-binding fragments (Fabs), and bi-specific antibodies. The anti-PD-1 and anti-PD-L1 therapeutic agents of the present disclosure also include, without limitation, other types of molecules capable of binding to and inhibiting an immune checkpoint function of PD-1 and/or PD-L1, respectively, such as polypeptides or proteins, including without limitation, human polypeptides or proteins, humanized polypeptides or proteins, or humanized polypeptides or proteins, and fusion proteins or fusion polypeptides, or small molecule inhibitors.

Immunotherapies are typically administered intravenously (i.v.). For example, both anti-PD-1 and anti-CTLA-4 immunotherapies are typically administered i.v. and have been shown to induce anti-tumor responses in patients with cancers, including melanoma, non-small cell lung cancer, and renal cell carcinomas. Because anti-CTLA-4 monotherapy is associated with lower response rates and higher rates of severe Grade 3-4 toxicities than anti-PD-1 monotherapy, anti-PD-1 monotherapy has become the preferred immunotherapy therapy in patients with advanced melanoma. Yet even for immunogenic melanoma, only 50% of patients are responsive to anti-PD-1 monotherapy. For these patients, combination of anti-CTLA-4 and anti-PD-1 therapies have been shown to have complementary activity of up to 50-60% response rates in advanced Stage III or IV melanoma, but disappointingly, they act synergistically to amplify immune-related adverse events (irAEs) and severe toxicity in up to 60% of all patients. Using analysis of outcome data from the CheckMate-067 trial, Oh, et al. argue that the elevated costs associated with irAEs due to anti-CTLA-4 in combination with anti-PD-1 antibody render it cost-ineffective despite the benefits of improved disease-free survival in responsive cancers. Maximizing exposure of anti-PD-1 and/or anti-PD-L1 therapeutic agents in TDLNs where cytotoxic T cells are activated against tumor Ags may improve anti-tumor responses, as well as minimize dose dependent irAEs.

In embodiments described herein, lymphatic delivery of checkpoint inhibitors including anti-PD-1 and/or anti-PD-L1 therapeutic agents may improve anti-tumor responses and alleviate irAEs in patients with cancers over that currently treated by conventional i.v. infusion.

PD-1 is expressed by T-cells in TDLNs and additionally PD-L1 is expressed by tumors to tolerize and limit T cell effector function in the tumor microenvironment. Accordingly, in some embodiments, lymphatic delivery of anti-PD-1 or anti-PD-L1 therapeutic agent may selectively remove mechanisms for inducing tolerance to tumor Ags within the TDLN or inhibition of T-cells by the tumors. In some embodiments, since lymph ultimately empties in the blood circulation, lymphatic delivery may also remove tolerance acquisition at peripheral LNs and other tumor sites, such as secondary tumor sites.

In some embodiments, due to enhanced exposure to drug targets, lymphatic delivery may allow a reduction of dose and may reduce dose-dependent irAEs that limit clinical use of anti-PD-1 and/or anti-PD-L1 therapeutic agents.

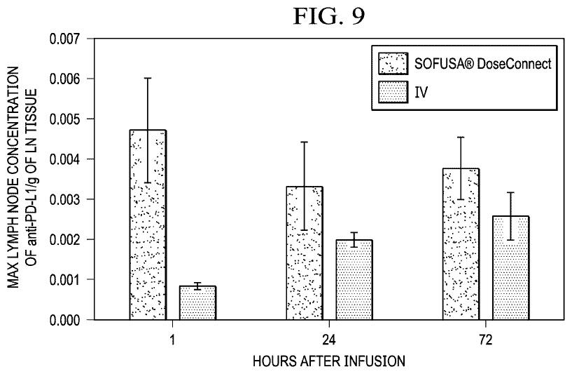

Lymph nodes (LNs) are part of the open, unidirectional lymphatic vasculature. Lymph nodes contain both T and B lymphocytes in addition to other cells associated with the immune system. The entry point for capillary filtrate, macromolecules, and immune cells is at the “initial” lymphatics that (i) lie immediately below the epidermis, (ii) surround the periphery of all organs, and (iii) can be formed at the tumor periphery through the process of tumor lymphangiogenesis (see FIG. 6). These “initial lymphatics” are immature capillaries without a basement membrane and with loose endothelial cell tight junctions that open and close to uniquely allow entry of waste materials and immune cells. From the initial lymphatics, lymph drains through mature, conducting lymphatic vessels that consist of a series of vessel segments bounded by valves and lined with smooth muscle cells that contract to actively propel lymph (often against gravity) to regional LN basins. Following transport through chains of downstream LNs via afferent and efferent lymphatic vessels, lymph is deposited into the blood vasculature via the subclavian vein. Monoclonal antibody access to the regional lymphatics following i.v. administration can occur through the high endothelial venules (HEVs) of LNs that are exclusive entryways for naïve T and B cell entry. In addition, antibodies that have extravasated from the blood vasculature may also be taken up by initial lymphatics for delivery to regional LNs. However, because lymph drains into the blood vasculature, comparatively large i.v. doses may be needed to reach drug targets associated with tumor Ags presentation within tumor draining LNs (see FIG. 6 inset A). Systemic i.v. administration of immune checkpoint inhibitors such as anti-PD-1 or anti-PD-L1 therapeutic agent can also result in activated naïve-T cells in non-tumor draining LNs where non-tumor, self Ags rather than tumor Ags are presented (see FIG. 6, inset B), and may lead to irAEs.

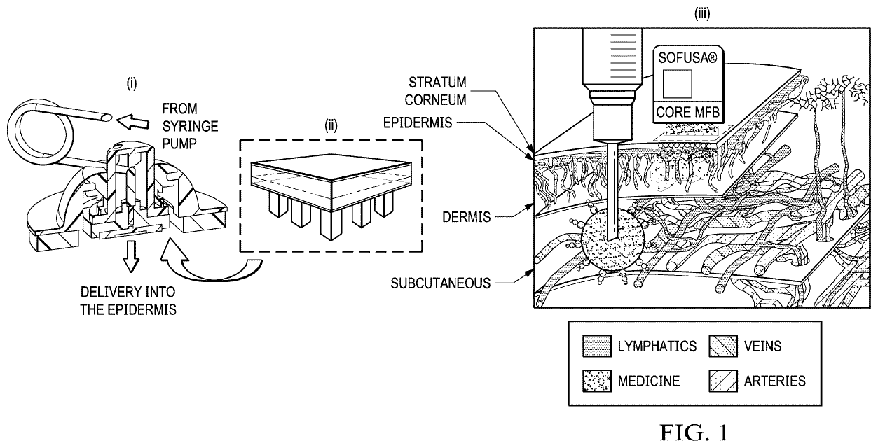

Administration of biologics directly into the lymphatics is challenging, with intradermal (i.d.) or Mantoux administration offering the most accessible entry point into the initial lymphatics below the epidermis (see FIG. 1). Unfortunately, the small sub-epidermal volume can accommodate ˜100 μL and ˜50 μL of injected fluid in humans and in rodent models, respectively, which limits the therapeutic dose of lymphatically delivered biologics in both preclinical and clinical investigations. Deeper, subcutaneous (s.c.) or intramuscular (i.m.) administration below the sub-epidermal space is inefficiently taken up by initial lymphatics and has reduced bioavailability due to off-target drug uptake and cellular processing.

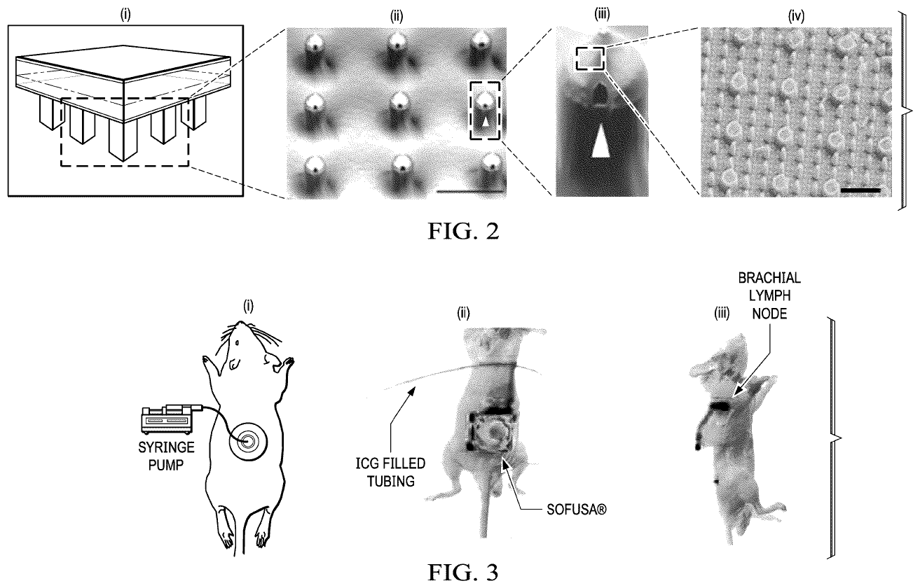

Many medical conditions benefit from having a steady state concentration of the active therapeutic agents for an extended period of time. Lymphatic delivery apparatuses are capable of administering therapeutic agents at a substantially constant rate over an extended period of time. Some devices are capable of delivering a therapeutic agent directly into the lymphatic system of a patient. One such device is the SOFUSA® (Sorrento Therapeutics, Inc. Corporation, Delaware) drug delivery platform. SOFUSA® is a nanotopography-based lymphatic delivery system. In one embodiment, the SOFUSA® lymphatic infusion device includes a single-use, 66 mm² array of 100 microneedles of 110 μm diameter, 350 μm long, and with a 30 μm hole located off-center (see FIG. 2). A 6-micron polyether ketone nanotopographical film heat-formed over each microneedle provides the nanotopographical features. These features have been shown to reversibly remodel tight junction proteins initiated via integrin binding to the nanotopography structures potentially augmenting uptake into the initial lymphatics. In both animals and humans, the device is first affixed via adhesive onto the tissue site of infusion, and then a calibrated applicator pushes the microneedle array into the sub-epidermal space where the initial lymphatics are located. A calibrated syringe pump then delivers drug into the microfluidic chamber, through the microneedle array, and into the sub-epidermal space in volumes not otherwise achievable through i.d injections (see FIG. 1).

SOFUSA® infuses drug within the sub-epidermis space and therefore accesses capillaries of both the hemovascular and lymphovascular systems. Many factors including size, composition, dose, surface charge, and molecular weight affect uptake into lymphatic and/or blood capillaries. For example, large particles, immune cells, and macromolecules are primarily taken up by lymphatic capillaries, while small particles and molecules less than 20 kDa can be absorbed by blood capillary networks. Glycocalyx on the luminal sides of blood vessels and capillaries is responsible for a force opposing capillary pressure and inhibits re-absorption of fluid into the venous vasculature. Thus, while the blood capillaries are intact and comparatively impermeable in the sub-epidermal space, the “initial lymphatics” represent immature capillaries without a basement membrane. They have “loose” lymphatic endothelial cell tight junctions that open and close via fibrils to uniquely allow entry of macromolecules, waste products, and immune cells. It has been estimated that as much as 12 liters of capillary filtrate (carrying small solutes and macromolecules) is collected from peripheral tissues by the initial lymphatics and returned to the blood vasculature. Because lymph drains to the blood vasculature, pharmacokinetic profiling in serum provides a measure of effectiveness of delivery through the lymphatics to the blood vasculature.

Metastasis is thought to be directly or indirectly responsible for more than 90% of all cancer deaths, and the lymphatic system is thought to play a significant role in cancer metastasis. Malignant cells may enter the lymphatic system and are captured by lymph nodes where secondary tumors can be produced. The lymphatic system is also often involved in the spread of tumors to other parts of the body (i.e., metastasis). Consequently, there is need for a method of preventing or reducing the spread of malignant cells via the lymphatic system.

In the methods disclosed herein, two different exemplary modes for delivering an anti-PD-1 or an anti-PD-L1 therapeutic agent to a patient are envisioned. In one mode, the target for the therapeutic agent is clearly identified, and the device comprising a plurality of microneedles is placed such that the anti-PD-1 or anti-PD-L1 therapeutic agent is administered to the lymphatic system of the patient so that the therapeutic agent is carried by the lymph vessels directly to that target. The target may be, for example, a solid tumor. In this case, while some systemic exposure will occur, the administration is much more regionalized.

In the second mode, the therapeutic target or exact location of the target may be unknown or less clearly defined. In such case, delivery of the therapeutic agent is into the lymphatic system of the patient, and the anti-PD-1 or anti-PD-L1 therapeutic agent is intended to traverse the lymphatic system to either the right lymphatic duct or the thoracic duct. The therapeutic agent then enters the circulatory system of the patient leading to systemic exposure to the agent. For example, if a solid tumor has metastasized, the location of secondary sites for these cancer cells may not be known. Although the therapeutic agent may traverse certain lymph nodes before reaching either of the draining ducts, the administration is considered to result in systemic exposure. As such, one skilled in the art can apply methods disclosed herein to provide targeted, regional administration of a therapeutic agent or more widespread systemic administration. A medical professional can determine which mode of administration is appropriate for an individual patient being treated with an anti-PD-1 therapeutic agent or an anti-PD-L1 therapeutic agent and place the device or devices accordingly.

In some aspects, the therapeutic target is a lymph node, a lymph vessel, an organ that is part of the lymphatic system or a combination thereof. In some aspects, the therapeutic target is a lymph node. In some aspects, the therapeutic target is a specific lymph node as described elsewhere herein. In certain embodiments, the target is a tumor.

In some embodiments, delivery of the therapeutic agent to the lymphatic system is delivery into the vessels of the lymphatic vasculature, the lymph nodes as described elsewhere herein, or both. In some aspects, delivery is to the superficial lymph vessels. In yet another aspect, delivery is to one or more lymph nodes. The specific target for delivery will be based on the medical needs of the patient.

In some embodiments, one or more devices described herein may be used to administer the anti-PD-1 or anti-PD-L1 therapeutic agent to a patient. In patients where more than one device is used to deliver the therapeutic agent to a plurality of locations on the body of a patient, the overall dose of the therapeutic agent at each location must be carefully adjusted such that the patient receives a therapeutically effective combined dose of the therapeutic agent. Being able to more selectively target specific locations in or on the body of a patient more precisely may mean that a lower dose is required at each specific location. In some embodiments, the dose administered to target one or more locations on the body of a patient is lower than a dose administered by other routes, including intravenous and subcutaneous administration.

In some embodiments, the anti-PD-1 or anti-PD-L1 therapeutic agent may be administered to one or more lymph nodes. In some embodiments, the anti-PD-1 or anti-PD-L1 therapeutic agent may be administered to one or more lymph vessels. In some embodiments, the anti-PD-1 or anti-PD-L1 therapeutic agent may be administered to lymph vessels that are proximal to a tumor. In some embodiments, the anti-PD-1 or anti-PD-L1 therapeutic agent may be administered to lymph vessels that are distal to a tumor.

Because the lymph fluid circulates throughout the body of a patient in a similar manner to blood in the circulatory system, any single position in the lymphatic vasculature can be upstream or downstream relative to another position. As used herein in reference to the lymphatic vasculature, the term “downstream” refers to a position in the lymphatic system closer (as the fluid travels through the vessels in a patient) to either the right lymphatic duct or the thoracic duct relative to the reference position (e.g., a tumor or internal organ or a joint). As used herein, the term “upstream” refers to a position in the lymphatic system that is farther from the right lymphatic duct or the thoracic duct relative to the reference position. Because the direction of fluid flow in the lymphatic system can be impaired or reversed due to the medical condition of the patient, the terms “upstream” and “downstream” do not specifically refer to the direction of fluid flow in the patient undergoing medical treatment. They are positional terms based on their physical position relative to the draining ducts as described.

In some embodiments, the therapeutic agent, such as an anti-PD-1 or anti-PD-L1 therapeutic agent is delivered to the interstitium of the patient, e.g., to a space between the skin and one or more internal structures, such as an organ, muscle, or vessel (artery, vein, or lymph vessel), or any other spaces within or between tissues or parts of an organ. In still yet another embodiment, the anti-PD-1 or anti-PD-L1 therapeutic agent is delivered to both the interstitium and the lymphatic system. In embodiments where the anti-PD-1 or anti-PD-L1 therapeutic agent is delivered to the interstitium of the patient, it may not be necessary to locate the lymph nodes or lymphatic vasculature of the patient before administering the anti-PD-1 or anti-PD-L1 therapeutic agent.

In some embodiments, a method of treating cancer in a patient is described. The method includes placing a device comprising a plurality of microneedles on the skin of the patient proximate to a first position under the skin of the patient, wherein the first position is proximate to lymph vessels and/or lymph capillaries in the patient's lymphatic system, and wherein the microneedles have a surface comprising nanotopography. The method also includes inserting the plurality of microneedles into the patient to a depth whereby at least the epidermis is penetrated and an end of at least one of the microneedles is proximate to the first position, and administering via the plurality of microneedles to the first position an effective amount of an anti-PD-1 therapeutic agent or an effective amount of an anti-PD-L1 therapeutic agent, thereby treating the cancer. In some embodiments, the present disclosure also relates to devices and/or systems described herein configured for lymphatic delivery of an anti-PD-1 or anti-PD-L1 therapeutic agent, wherein the device and/or system comprises an amount of an anti-PD-1 therapeutic agent or anti-PD-L1 therapeutic agent effective for treating cancer in a patient.

In some embodiments, a method of preventing or reducing cancer metastasis in a patient is described. The method includes locating at least one lymph node in the patient that intervenes in the lymphatic system between a solid cancer tumor and a draining duct, placing a device comprising a plurality of microneedles on the skin of the patient proximate to a first position under the skin of the patient located between the intervening lymph node and the solid cancer tumor, wherein the first position is proximate to lymph vessels and/or lymph capillaries in the patient's lymphatic system, and wherein the microneedles have a surface comprising nanotopography, inserting the plurality of microneedles into the patient to a depth whereby at least the epidermis is penetrated and an end of at least one of the microneedles is proximate to the first position; and administering via the plurality of microneedles to the first position a therapeutically effective amount of an anti-PD-1 therapeutic agent or an anti-PD-L1 therapeutic agent that is effective for preventing or reducing metastasis of the solid cancer tumor. In some embodiments, the present disclosure also relates to devices and/or systems described herein configured for lymphatic delivery of an anti-PD-1 or anti-PD-L1 therapeutic agent, wherein the device and/or system comprises an amount of an anti-PD-1 therapeutic agent or anti-PD-L1 therapeutic agent effective for preventing or reducing cancer metastasis in a patient.

Accordingly, in some embodiments, a method of preventing or reducing cancer metastasis in a patient is described. The method includes locating a solid cancer tumor in the patient, locating at least one lymph node in the patient that intervenes in the lymphatic system between the solid cancer tumor and a draining duct, placing a device that comprises a plurality of microneedles on the skin of the patient at a first location on the skin of the patient that is proximate to lymph capillaries and/or lymph vessels that flow into the intervening lymph node, wherein the microneedles have a surface comprising nanotopography, inserting the plurality of microneedles into the patient to a depth whereby at least the epidermis is penetrated, and administering via the plurality of microneedles to the lymph capillaries and/or lymph vessels that flow into the intervening lymph node a therapeutically effective amount of an anti-PD-1 therapeutic agent or an anti-PD-L1 therapeutic agent that is effective in preventing or reducing cancer metastasis. In some embodiments, the present disclosure also relates to devices and/or systems described herein configured for lymphatic delivery of an anti-PD-1 or anti-PD-L1 therapeutic agent, wherein the device and/or system comprises an amount of an anti-PD-1 therapeutic agent or anti-PD-L1 therapeutic agent, effective for preventing or reducing cancer metastasis in a patient.

In some embodiments, the cancer comprises a tumor. In some embodiments, the lymph node is a tumor draining lymph node. Tumor draining lymph node refers to a lymph node that is downstream from a solid cancer tumor and is the first lymph node impacted by metastasis of the tumor. The first lymph node affected by metastasis is often referred to as the sentinel lymph node.

Because metastasis can be a systemic issue for a patient rather than just a localized one, in some embodiments the device is placed on the patient to effect systemic delivery of the anti-PD-1 therapeutic agent or anti-PD-L1 therapeutic agent, rather than just targeted delivery to an identified lymph node. In some embodiments, the device is placed such that the anti-PD-1 therapeutic agent or anti-PD-L1 therapeutic agent is not targeting a specific lymph node, although it may traverse one or more lymph nodes after administration; the device is placed with the expectation that the anti-PD-1 therapeutic agent or anti-PD-L1 therapeutic agent will enter the circulatory system of the patient after traversing the lymphatic vasculature leading to systemic exposure to the anti-PD-1 therapeutic agent or anti-PD-L1 therapeutic agent. This type of administration is intended to treat metastasized cancer cells that have moved past the local environment of the primary solid cancer tumor. Such metastasized cancer cells may not yet be exhibiting symptoms in that new location, but may do so if left untreated.

In some embodiments, the device is placed, relative to the tumor, distal to the draining duct. In some embodiments, at least one lymph node in the patient intervenes in the lymphatic system between the tumor and a draining duct, and the first position is located between the intervening lymph node and the tumor. In some embodiments, the device is placed at a location on the skin of the patient having lymphatic capillaries and/or vessels that flow directly into the intervening lymph node without first passing through any prior lymph node.

In some embodiments, disclosed herein is a method for treating a solid cancer tumor in a patient. The method generally comprises locating the solid cancer tumor in the patient; locating a position in the lymphatic system of the patient that is upstream of the solid cancer tumor; placing a device comprising a plurality of microneedles on the skin of the patient proximate to a first position under the skin of the patient located proximate to the position in the lymphatic system of the patient that is upstream of the solid cancer tumor, wherein the first position is proximate to lymph vessels and/or lymph capillaries that are upstream of the solid cancer tumor, and wherein the microneedles have a surface comprising nanotopography; inserting the plurality of microneedles into the patient to a depth whereby at least the epidermis is penetrated; and administering via the plurality of microneedles to the first position a therapeutically effective amount of an anti-PD-1 therapeutic agent or an anti-PD-L1 therapeutic agent, for example that is effective for preventing or reducing metastasis of the solid cancer tumor. In some aspects, the position in the lymphatic system of the patient to which the anti-PD-1 therapeutic agent or anti-PD-L1 therapeutic agent is delivered is downstream of the solid cancer tumor. In some embodiments, the present disclosure also relates to devices and/or systems described herein configured for lymphatic delivery of an anti-PD-1 or anti-PD-L1 therapeutic agent, wherein the device and/or system comprises an amount of an anti-PD-1 therapeutic agent or anti-PD-L1 therapeutic agent effective for treating a solid cancer tumor in a patient.

In some embodiments, administering is done to the lymph vessels upstream to the solid cancer tumor. In other embodiments, administering is done to both the lymph nodes and lymph vessels upstream of the solid cancer tumor. In some aspects, it may not be necessary to locate a lymph node upstream of the tumor before administering the anti-PD-1 therapeutic agent or anti-PD-L1 therapeutic agent to the patient. In some embodiments, the device is placed distal to the draining duct relative to the solid cancer tumor. In yet another aspect, the device is proximal to the draining duct relative to the solid cancer tumor.

Because cancer and other medical conditions can damage the lymphatic system of a patient, the flow of fluid in the lymphatic system can be impaired or even reversed (called backflow). This can lead to swelling in the surrounding tissues and organs of the patient. In some aspects, the device is placed such that backflow in the lymphatic system transports the anti-PD-1 therapeutic agent or anti-PD-L1 therapeutic agent to the targeted location. For example, in a properly functioning lymphatic system, the downstream position relative to a solid cancer tumor would not transport the anti-PD-1 therapeutic agent or anti-PD-L1 therapeutic agent directly into the tumor. However, in an impaired lymphatic system, backflow from a downstream position relative to the solid cancer tumor would transport the anti-PD-1 therapeutic agent or anti-PD-L1 therapeutic agent directly to the tumor. A medical professional skilled in the art understands the manner by which the lymphatic system functions and will make treatment decisions for the patient based on that knowledge.

In some aspects, the device is placed at a location on the skin of the patient such that the lymph vessels and/or capillaries flow directly into a specifically targeted lymph node without first passing through the solid cancer tumor or any other lymph nodes. In this instance, the anti-PD-1 therapeutic agent or anti-PD-L1 therapeutic agent, after administration, would enter the lymph vessels of the patient and flow directly into the targeted lymph node. In yet another aspect, there may be lymph nodes between the site of administration and the targeted lymph nodes. One nonlimiting example when this occurs is if the targeted lymph node is deep within the body of the patient and there are no lymph vessels near the skin of the patient that flow directly into the targeted lymph node.

It is known that certain types of cancer often metastasize to specific lymph nodes, and the placement of the device may be made on this basis. For example, oral and pharynx cancers, in addition to those of the head and neck, metastasize to the jugular lymph node chain, the cervical lymph nodes and the supraclavicular lymph nodes; many skin cancers (e.g., melanomas) metastasize to the draining axillary and/or inguinal lymph node basins depending on the location of the cancer; breast cancer metastasizes to the axillary, internal mammary and supraclavicular lymph nodes; prostate cancer metastasizes to the lumbar, inguinal and peritoneal lymph nodes; brain and central nervous system cancers metastasize into the jugular, cervical and lumbar lymph nodes; ovarian cancers metastasize to the retroperitoneal (pelvic and/or para-aortic) lymph nodes; cancer in the genitals of a patient metastasize to the lumbar lymph nodes, the inguinal lymph nodes, and the peritoneal lymph nodes.

The specific lymph node targeted for delivery of the therapeutic agent is based on any reasonable criteria based on the medical needs and condition of the patient. For example, a lymph node biopsy may be performed to determine if metastatic cancer cells are present in a specific lymph node. Alternatively, a lymph node may be selected based on its location relative to a previously located tumor in the body of a patient. In some embodiments, the lymph node is selected because it is downstream from the solid cancer tumor. Placing the device in a position to target the downstream lymph nodes would affect metastatic cancer cells that are in those lymph nodes and reduce the likelihood of their spreading to other parts of the body. Alternatively, the device may be placed upstream of the tumor in order to take advantage of tumor-induced lymphangiogenesis that often occurs with solid cancer tumors. In this arrangement, the therapeutic agent would flow directly into the tumor thereby more effectively targeting the tumor.

In some embodiments, the cancer is a cancer of the head and neck, and the lymph nodes are selected from the group consisting of the jugular lymph nodes, the cervical lymph nodes, the supraclavicular lymph nodes, and combinations thereof. In some embodiments, the cancer is an oral cavity cancer, and the lymph nodes are selected from the group consisting of the jugular lymph node chain, the cervical lymph nodes, the supraclavicular lymph nodes, and combinations thereof. In some embodiments, the cancer is a cancer of the pharynx, and the lymph nodes are selected from the group consisting of the jugular lymph node chain, the cervical lymph nodes, the supraclavicular lymph nodes, and combinations thereof. In some embodiments, the cancer is a melanoma, and the lymph nodes are selected from the group consisting of axillary lymph nodes, inguinal lymph nodes, jugular lymph nodes, cervical lymph nodes, supraclavicular lymph nodes, and combinations thereof. In some embodiments, the cancer is breast cancer, and lymph nodes are selected from the group consisting of the axillary lymph nodes, the internal mammary lymph nodes, the supraclavicular lymph nodes and combinations thereof. In some embodiments, the cancer is prostate cancer, and the lymph nodes are selected from the group consisting of the lumbar lymph nodes, the inguinal lymph nodes, the peritoneal lymph nodes and combinations thereof. In some embodiments, the cancer is in the genital system of the patient and the lymph nodes are selected from the group consisting of the lumbar lymph nodes, the inguinal lymph nodes, the peritoneal lymph nodes and combinations thereof.

In some embodiments, the amount of the anti-PD-1 therapeutic agent or anti-PD-L1 therapeutic agent required to target the metastatic cancer cells or the tumor is lower than the amount given by other routes of administration. A lower dose that is still therapeutically effective may reduce or eliminate side effects leading to a more positive patient outcome.

In some embodiments, the anti-PD-1 therapeutic agent may be nivolumab (PD-1 inhibitor; sold under the tradename of Opdivo®, Bristol-Myers Squibb), pembrolizumab (PD-1 inhibitor; sold under the tradename of Keytruda®, Merck Sharp & Dohme, New Jersey), cemiplimab (sold under the tradename of Libtayo®, Regeneron Pharmaceuticals, New York), Spartalizumab (PDR001; Novartis), Camrelizumab (SHR1210; Jiangsu HengRui Medicine Co., Ltd.), Sintilimab (IBI308; Innovent and Eli Lilly), Tislelizumab (BGB-A317; BeiGene and Celgene Corp.), Toripalimab (JS 001; Beijing Cancer Hospital), AMP-224 (GlaxoSmithKline), AMP-514 (GlaxoSmithKline), anti-PD1 monoclonal antibody STI-A1110 (Sorrento Pharmaceuticals; also referred to herein as STI-2949 and also referred to as RGIH10 in International Application PCT/US2014/040420 filed May 31, 2014 and published as WO 2014/194302 A2, the contents of which are incorporated by reference herein in it's entirety, AUNP12 (a 29-mer peptide PD-1/PD-L1 inhibitor developed by Aurigene and Laboratoires Pierre Fabre), or a biosimilar thereof, or a bioequivalent thereof.

In some embodiments, the anti-PD-1 therapeutic agent comprises a heavy chain variable domain, or one or more of heavy chain complimentarity determining regions (“CDR”) 1, 2, and 3 (“CDR-H1”, “CDR-H2”, and “CDR-H3”, respectively) contained in such heavy chain variable domain, having at least 80%, at least 81%, at least 82%, at least 83%, at least 84%, at least 85, 86%, at least 87%, at least 88%, at least 89%, at least 90%, at least 91%, at least 92%, at least 93%, at least 94%, at least 95%, at least 96%, at least 97%, at least 98%, at least 99%, at least 90%, at least 95%, at least 96%, at least 97%, at least 98%, at least 99%, or at least 100% identity to an amino acid sequence selected from the group consisting of SEQ ID NO. 1, SEQ ID NO. 3, SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 23, SEQ ID NO. 25, SEQ ID NO. 27, SEQ ID NO. 29, SEQ ID NO. 31, SEQ ID NO. 33, SEQ ID NO. 35, SEQ ID NO. 37, SEQ ID NO. 38, SEQ ID NO. 39, SEQ ID NO. 40, SEQ ID NO. 41, SEQ ID NO. 42, SEQ ID NO. 43, SEQ ID NO. 44, and combinations thereof. In some embodiments, the anti-PD-1 therapeutic agent comprises a light chain variable domain, or one or more of light chain complimentarity determining regions (“CDR”) 1, 2, and 3 (“CDR-L1”, “CDR-L2”, and “CDR-L3”, respectively) contained in such light chain variable domain, having at least 80%, at least 81%, at least 82%, at least 83%, at least 84%, at least 85, 86%, at least 87%, at least 88%, at least 89%, at least 90%, at least 91%, at least 92%, at least 93%, at least 94%, at least 95%, at least 96%, at least 97%, at least 98%, at least 99%, at least 90%, at least 95%, at least 96%, at least 97%, at least 98%, at least 99%, or at least 100% identity to an amino acid sequence selected from the group consisting of SEQ ID NO. 2, SEQ ID NO. 4, SEQ ID NO. 6, SEQ ID NO. 8, SEQ ID NO. 10, SEQ ID NO. 12, SEQ ID NO. 14, SEQ ID NO. 16, SEQ ID NO. 18, SEQ ID NO. 20, SEQ ID NO. 22, SEQ ID NO. 24, SEQ ID NO. 26, SEQ ID NO. 28, SEQ ID NO. 30, SEQ ID NO. 32, SEQ ID NO. 34, SEQ ID NO. 36, and combinations thereof.

In some embodiments, the anti-PD-1 therapeutic agent comprises: a heavy chain variable domain, or one or more of heavy chain complimentarity determining regions (“CDR”) 1, 2, and 3 (“CDR-H1”, “CDR-H2”, and “CDR-H3”, respectively) contained in such heavy chain variable domain, having at least 80%, at least 81%, at least 82%, at least 83%, at least 84%, at least 85, 86%, at least 87%, at least 88%, at least 89%, at least 90%, at least 91%, at least 92%, at least 93%, at least 94%, at least 95%, at least 96%, at least 97%, at least 98%, at least 99%, at least 90%, at least 95%, at least 96%, at least 97%, at least 98%, at least 99%, or at least 100% identity to an amino acid sequence selected from the group consisting of SEQ ID NO. 1, SEQ ID NO. 3, SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 23, SEQ ID NO. 25, SEQ ID NO. 27, SEQ ID NO. 29, SEQ ID NO. 31, SEQ ID NO. 33, SEQ ID NO. 35, SEQ ID NO. 37, SEQ ID NO. 38, SEQ ID NO. 39, SEQ ID NO. 40, SEQ ID NO. 41, SEQ ID NO. 42, SEQ ID NO. 43, SEQ ID NO. 44, and combinations thereof; and a light chain variable domain, or one or more of light chain complimentarity determining regions (“CDR”) 1, 2, and 3 (“CDR-L1”, “CDR-L2”, and “CDR-L3”, respectively) contained in such light chain variable domain, having at least 80%, at least 81%, at least 82%, at least 83%, at least 84%, at least 85, 86%, at least 87%, at least 88%, at least 89%, at least 90%, at least 91%, at least 92%, at least 93%, at least 94%, at least 95%, at least 96%, at least 97%, at least 98%, at least 99%, at least 90%, at least 95%, at least 96%, at least 97%, at least 98%, at least 99%, or at least 100% identity to an amino acid sequence selected from the group consisting of SEQ ID NO. 2, SEQ ID NO. 4, SEQ ID NO. 6, SEQ ID NO. 8, SEQ ID NO. 10, SEQ ID NO. 12, SEQ ID NO. 14, SEQ ID NO. 16, SEQ ID NO. 18, SEQ ID NO. 20, SEQ ID NO. 22, SEQ ID NO. 24, SEQ ID NO. 26, SEQ ID NO. 28, SEQ ID NO. 30, SEQ ID NO. 32, SEQ ID NO. 34, SEQ ID NO. 36, and combinations thereof.

In some embodiments, the anti-PD-1 therapeutic agent comprises a heavy chain variable domain sequence and a light chain variable domain sequence (heavy chain variable domain sequence/light chain variable domain sequence) selected from the group consisting of SEQ ID NO. 1/SEQ ID NO. 2 (called GA1 herein), SEQ ID NO. 3/SEQ ID NO. 4 (called GA2 herein), SEQ ID NO. 5/SEQ ID NO. 6 (called GB1 herein), SEQ ID NO. 7/SEQ ID NO. 8 (called GB6 herein), SEQ ID NO. 9/SEQ ID NO. 10 (called GH1 herein), SEQ ID NO. 11/SEQ ID NO. 12 (called A2 herein), SEQ ID NO. 13/SEQ ID NO. 14 (called C7 herein), SEQ ID NO. 15/SEQ ID NO. 16 (called H7 herein), SEQ ID NO. 17/SEQ ID NO. 18 (called SH-A4 herein), SEQ ID NO. 19/SEQ ID NO. 20 (called SH-A9 herein), SEQ ID NO. 21/SEQ ID NO. 22 (called RG1B3 herein), SEQ ID NO. 23/SEQ ID NO. 24 (called STI-2949, STI-A1110, or RG1H10 herein, used interchangeably throughout), SEQ ID NO. 25/SEQ ID NO. 26 (called RG1H11 herein), SEQ ID NO. 27/SEQ ID NO. 28 (called RG2H7 herein), SEQ ID NO. 29/SEQ ID NO. 30 (called RG2H10 herein), SEQ ID NO. 31/SEQ ID NO. 32 (called RG3E12 herein), SEQ ID NO. 33/SEQ ID NO. 34 (called RG4A6 herein), SEQ ID NO. 35/SEQ ID NO. 36 (called RG5D9 herein), SEQ ID NO. 37/SEQ ID NO. 24 (called RG1H10-H2A-22-15 herein), SEQ ID NO. 38/SEQ ID NO. 24 (called RG1H10-H2A-27-25 herein), SEQ ID NO. 39/SEQ ID NO. 24 (called RG1H10-3C herein), SEQ ID NO. 40/SEQ ID NO. 24 (called RG1H10-16C herein), SEQ ID NO. 41/SEQ ID NO. 24 (called RG1H10-17C herein), SEQ ID NO. 42/SEQ ID NO. 24 (called RG1H10-19C herein), SEQ ID NO. 43/SEQ ID NO. 24 (called RG1H10-21C herein), SEQ ID NO. 44/SEQ ID NO. 24 (called RG1H10-23C2 herein), and combinations thereof.

In some embodiments, the anti-PD-L1 therapeutic agent may be atezolizumab (PD-L1 inhibitor; sold under the tradename of Tecentriq®, Genentech, Inc., Delaware), avelumab (sold under the tradename of Bavencio®, Merck, Germany), durvalumab (sold under the tradename of Imfinzi®, AstraZeneca, Sweden), KN035 (Alphamab and 3D Medicines), AUNP12 (a 29-mer peptide PD-1/PD-L1 inhibitor developed by Aurigene and Laboratoires Pierre Fabre), CA-170 (Aurigene/Curis; a small molecule PD-L1 inhibitor), BMS-986189 (Bristol-Myers Squibb; a macrocyclic peptide), or a biosimilar thereof, or a bioequivalent thereof.

In some embodiments, the anti-PD-L1 therapeutic agent comprises a heavy chain variable domain, or one or more of heavy chain complimentarity determining regions (“CDR”) 1, 2, and 3 (“CDR-H1”, “CDR-H2”, and “CDR-H3”, respectively) contained in such heavy chain variable domain, having at least 80%, at least 81%, at least 82%, at least 83%, at least 84%, at least 85, 86%, at least 87%, at least 88%, at least 89%, at least 90%, at least 91%, at least 92%, at least 93%, at least 94%, at least 95%, at least 96%, at least 97%, at least 98%, at least 99%, at least 90%, at least 95%, at least 96%, at least 97%, at least 98%, at least 99%, or at least 100% identity to an amino acid sequence disclosed in WO 2013/181634, the contents of which are hereby incorporated by reference in its entirety. In some embodiments, the anti-PD-L1 therapeutic agent comprises a light chain variable domain, or one or more of light chain complimentarity determining regions (“CDR”) 1, 2, and 3 (“CDR-L1”, “CDR-L2”, and “CDR-L3”, respectively) contained in such light chain variable domain, having at least 80%, at least 81%, at least 82%, at least 83%, at least 84%, at least 85, 86%, at least 87%, at least 88%, at least 89%, at least 90%, at least 91%, at least 92%, at least 93%, at least 94%, at least 95%, at least 96%, at least 97%, at least 98%, at least 99%, at least 90%, at least 95%, at least 96%, at least 97%, at least 98%, at least 99%, or at least 100% identity to an amino acid sequence disclosed in WO 2013/181634 or US 2017/0218066, the contents of which are hereby incorporated by reference in their entireties.

In some embodiments, the anti-PD-L1 therapeutic agent comprises: a heavy chain variable domain, or one or more of heavy chain complimentarity determining regions (“CDR”) 1, 2, and 3 (“CDR-H1”, “CDR-H2”, and “CDR-H3”, respectively) contained in such heavy chain variable domain, having at least 80%, at least 81%, at least 82%, at least 83%, at least 84%, at least 85, 86%, at least 87%, at least 88%, at least 89%, at least 90%, at least 91%, at least 92%, at least 93%, at least 94%, at least 95%, at least 96%, at least 97%, at least 98%, at least 99%, at least 90%, at least 95%, at least 96%, at least 97%, at least 98%, at least 99%, or at least 100% identity to an amino acid sequence disclosed in WO 2013/181634, the contents of which are hereby incorporated by reference in its entirety; and a light chain variable domain, or one or more of light chain complimentarity determining regions (“CDR”) 1, 2, and 3 (“CDR-L1”, “CDR-L2”, and “CDR-L3”, respectively) contained in such light chain variable domain, having at least 80%, at least 81%, at least 82%, at least 83%, at least 84%, at least 85, 86%, at least 87%, at least 88%, at least 89%, at least 90%, at least 91%, at least 92%, at least 93%, at least 94%, at least 95%, at least 96%, at least 97%, at least 98%, at least 99%, at least 90%, at least 95%, at least 96%, at least 97%, at least 98%, at least 99%, or at least 100% identity to an amino acid sequence disclosed in WO 2013/181634 or US 2017/0218066, the contents of which are hereby incorporated by reference in their entireties.

In certain embodiments, a “therapeutic agent”, such as an anti-PD-1 therapeutic agent or an anti-PD-L1 therapeutic agent, may comprise and “antigen binding protein”. In certain embodiments a “therapeutic agent”, which may comprise an “antigen binding protein”, comprises a protein or an antigen binding portion or fragment thereof and/or, optionally, a scaffold or framework portion, that allows the antigen binding portion to adopt a conformation that promotes binding of the antigen binding protein to an antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold.

An antigen binding protein can have, for example, the structure of a naturally occurring immunoglobulin. An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

The variable regions of naturally occurring immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4. The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991. Other numbering systems for the amino acids in immunoglobulin chains include IMGT.RTM. (international ImMunoGeneTics information system; Lefranc et al, Dev. Comp. Immunol. 29:185-203; 2005) and AHo (Honegger and Pluckthun, J. Mol. Biol. 309(3):657-670; 2001).

Antibodies can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecfic antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen.

An “antibody” refers to an intact immunoglobulin or to an antigen binding portion thereof that competes with the intact antibody for specific binding, unless otherwise specified. Antigen binding portions may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

A Fab fragment is a monovalent fragment having the V_{L}, V_{H}, C_{L} and C_{H1} domains; a F(ab′)₂ fragment is a bivalent fragment having two Fab fragments linked by a disulfide bridge at the hinge region; a Fd fragment has the V_{H} and C_{H1} domains; an Fv fragment has the V_{L} and V_{H} domains of a single arm of an antibody; and a dAb fragment has a V_{H} domain, a V_{L} domain, or an antigen-binding fragment of a V_{H} or VL domain (U.S. Pat. Nos. 6,846,634; 6,696,245, US App. Pub. 20/0202512; 2004/0202995; 2004/0038291; 2004/0009507; 20 03/0039958, and Ward et al., Nature 341:544-546, 1989).

A single-chain antibody (scFv) is an antibody in which a V_{L} and a V_{H} region are joined via a linker (e.g., a synthetic sequence of amino acid residues) to form a continuous protein chain wherein the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83). Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48, and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

Complementarity determining regions (CDRs) and framework regions (FR) of a given antibody may be identified using the system described by Kabat et al. supra; Lefranc et al., supra and/or Honegger and Pluckthun, supra. One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

An antigen binding protein may have one or more binding sites. If there is more than one binding site, the binding sites may be identical to one another or may be different. For example, a naturally occurring human immunoglobulin typically has two identical binding sites, while a “bispecific” or “bifunctional” antibody has two different binding sites.

The term “human antibody” includes all antibodies that have one or more variable and constant regions derived from human immunoglobulin sequences. In one embodiment, all of the variable and constant domains are derived from human immunoglobulin sequences (a fully human antibody). These antibodies may be prepared in a variety of ways, examples of which are described below, including through the immunization with an antigen of interest of a mouse that is genetically modified to express antibodies derived from human heavy and/or light chain-encoding genes.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies. In one embodiment, one or more of the CDRs are derived from a human antibody. In another embodiment, all of the CDRs are derived from a human antibody. In another embodiment, the CDRs from more than one human anti-PD-1 antibodies are mixed and matched in a chimeric antibody. For instance, a chimeric antibody may comprise a CDR1 from the light chain of a first human anti-PD-1 antibody, a CDR2 and a CDR3 from the light chain of a second human anti-PD-1 antibody, and the CDRs from the heavy chain from a third anti-PD-1 antibody. Other combinations are possible. In another embodiment, the CDRs from more than one human anti-PD-L1 antibodies are mixed and matched in a chimeric antibody. For instance, a chimeric antibody may comprise a CDR1 from the light chain of a first human anti-PD-L1 antibody, a CDR2 and a CDR3 from the light chain of a second human anti-PD-L1 antibody, and the CDRs from the heavy chain from a third anti-PD-L1 antibody. Other combinations are possible.

Further, the framework regions may be derived from one of the same anti-PD-1 antibodies or anti-PD-L1 antibodies, respectively, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind PD-1 or PD-L1).

A “neutralizing antibody” or an “inhibitory antibody” is an antibody that inhibits the proteolytic activation of PD-1 or PD-L1 when an excess of the anti-PD-1 antibody or the anti-PD-L1 antibody reduces the amount of activation by at least about 20% using an assay such as those described herein in the Examples. In various embodiments, the antigen binding protein reduces the amount of amount of proteolytic activation of PD-1 or PD-L1 by at least 30%, 40%, 50%, 60%, 70%, 75%, 80%, 85%, 90%, 95%, 97%, 99%, and 99.9%.

Fragments or analogs of antibodies can be readily prepared by those of ordinary skill in the art following the teachings of this specification and using techniques known in the art. Preferred amino- and carboxy-termini of fragments or analogs occur near boundaries of functional domains. Structural and functional domains can be identified by comparison of the nucleotide and/or amino acid sequence data to public or proprietary sequence databases. Computerized comparison methods can be used to identify sequence motifs or predicted protein conformation domains that occur in other proteins of known structure and/or function. Methods to identify protein sequences that fold into a known three-dimensional structure are known. See, Bowie et al., 1991, Science 253:164.

A “CDR grafted antibody” is an antibody comprising one or more CDRs derived from an antibody of a particular species or isotype and the framework of another antibody of the same or different species or isotype.

A “multi-specific antibody” is an antibody that recognizes more than one epitope on one or more antigens. A subclass of this type of antibody is a “bi-specific antibody” which recognizes two distinct epitopes on the same or different antigens.

An antigen binding protein “specifically binds” to an antigen (e.g., human PD-1 or PD-L1) if it binds to the antigen with a dissociation constant of 1 nanomolar or less.

An “antigen binding domain,” “antigen binding region,” or “antigen binding site” is a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains.

An “epitope” is the portion of a molecule that is bound by an antigen binding protein (e.g., by an antibody). An epitope can comprise non-contiguous portions of the molecule (e.g., in a polypeptide, amino acid residues that are not contiguous in the polypeptide's primary sequence but that, in the context of the polypeptide's tertiary and quaternary structure, are near enough to each other to be bound by an antigen binding protein).

The “percent identity” of two polynucleotide or two polypeptide sequences is determined by comparing the sequences using the GAP computer program (a part of the GCG Wisconsin Package, version 10.3 (Accelrys, San Diego, Calif.)) using its default parameters.

If two or more devices are used, the anti-PD-1 therapeutic agent administered to the patient using the two or more devices may be the same or different. If two or more devices are used, the anti-PD-L1 therapeutic agent administered to the patient using the two or more devices may be the same or different. If two or more devices are used, the anti-PD-1 therapeutic agent and/or the anti-PD-L1 therapeutic agent administered to the patient using the two or more devices may each independently be the same or different.

In yet another aspect, two or more devices comprising a plurality of microneedles are used to administer a single anti-PD-1 therapeutic agent or anti-PD-L1 therapeutic agent. In this case each individual dose administered by each device may be smaller than a therapeutically effective dose, but the combined dose administered by the two or more devices is therapeutically effective.

When the methods, devices and/or systems disclosed herein are used to treat solid cancer tumors or treat, reduce or eliminate cancer metastasis, the cancer may be any type susceptible to treatment with an anti-PD-1 therapeutic agent or anti-PD-L1 therapeutic agent. The type of cancer includes, but is not limited to, Cutaneous T-cell lymphomas (CTCLs), adenoid cystic carcinoma, adrenal gland tumor, amyloidosis, anal cancer, ataxia-telangiectasia, atypical mole syndrome, beckwith wiedemann syndrome, bile duct cancer, birt hogg dube syndrome, bladder cancer, bone cancer, brain tumor, breast cancer, carcinoid tumor, carney complex, cervical cancer, colorectal cancer, ductal carcinoma, endometrial cancer, esophageal cancer, familial-adenomatous polyposis, gastric cancer, gastrontestinal stromal tumor, islet cell tumor, juvenile polyposis syndrome, Kaposi's sarcoma, kidney cancer, laryngeal cancer, liver cancer, lobular carcinoma, lung cancer, small cell lung cancer, Hodgkin's lymphoma, non-Hodgkin's lymphoma, lynch syndrome, malignant glioma, mastocytosis, melanoma, meningioma, multiple endocrine neoplasia type 1, multiple Endocrine Neoplasia type 2, multiple myeloma, myelodysplastic syndrome, Nasopharyngeal cancer, Neuroendocrine tumor, Nevoid basal cell carcinoma syndrome, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, pleuropulmonary blastoma, polycythemia vera, prostate cancer, renal cell cancer, retinoblastoma, salivary gland cancer, sarcoma, alveolar soft part and cardiac sarcoma, Kaposi sarcoma, skin cancer, small bowel cancer, small Intestine cancer, stomach cancer, testicular cancer, thymoma, thyroid cancer, turcot syndrome, uterine (endometrial) cancer, vaginal cancer, von-Hippel-Lindau syndrome, Wilms' tumor (childhood), xeroderma pigmentosum and combinations thereof. In some aspects, the cancer is selected from the group consisting of bladder cancer, breast cancer, cervical cancer, colorectal cancer, esophageal cancer, Hodgkin's lymphoma, non-Hodgkin's lymphoma, kidney cancer, lung cancer, small cell lung cancer, melanoma, oral cancer, pancreatic cancer, pancreatic neuroendocrine tumors, penile cancer, prostate cancer, renal cell cancer, stomach cancer, testicular cancer, thyroid cancer, uterine (endometrial) cancer, and vaginal cancer.

In some aspects, administering the anti-PD-1 therapeutic agent or anti-PD-L1 therapeutic agent is directly to a lymph node, a lymph vessel, an organ that is part of the lymphatic system or a combination thereof. In some aspects, administering is to a lymph node. In some aspects, administering is to a specific lymph node as described elsewhere herein. In yet another aspect, administering is to lymph vessels that are upstream of and known to flow into specific lymph nodes. In yet another aspect, administering is to lymph vessels that are upstream of and known to flow into a solid cancer tumor.

It is understood that when multiple doses of an anti-PD-1 therapeutic agent or anti-PD-L1 therapeutic agent are administered to a patient, each individual dose may not be therapeutically effective, but the combined doses are therapeutically effective. The combined doses that are therapeutically effective may be smaller than a therapeutically effective dose if the anti-PD-1 therapeutic agent or anti-PD-L1 therapeutic agent is administered by a different route (e.g., subcutaneous, intravenous, etc.).

In some embodiments, delivery of the anti-PD-1 therapeutic agent or anti-PD-L1 therapeutic agent to the lymphatic system is delivery into the lymphatic vasculature, the lymph nodes as described elsewhere herein, or both. In some aspects, delivery is to the superficial lymph vessels. In yet another aspect, delivery is to one or more lymph nodes. The specific target for delivery will be based on the medical needs of the patient. In one nonlimiting example, if a lymph node biopsy or other medical assessment (e.g., lymph node swelling) is found to be positive for possible metastatic cancer cells, then the device comprising a plurality of microneedles can be placed on the patient such that it delivers the anti-PD-1 therapeutic agent or anti-PD-L1 therapeutic agent directly to the lymph node. In another nonlimiting example, a sentinel lymph node biopsy is performed where the sentinel lymph nodes are selected based on the type of cancer and the assessment of a medical professional. Alternatively, the device can be placed upstream of the lymph node such that the anti-PD-1 therapeutic agent or anti-PD-L1 therapeutic agent is delivered to the lymph vessels that feed into the targeted lymph node. In some embodiments, two or more devices are used to target two or more different locations in the lymphatic system of the patient. In another nonlimiting example, the device is placed upstream of a solid cancer tumor such that the anti-PD-1 therapeutic agent or anti-PD-L1 therapeutic agent feeds directly into the tumor. In another example, the device is placed directly downstream from a solid cancer tumor such that the anti-PD-1 therapeutic agent or anti-PD-L1 therapeutic agent would traverse the same lymphatic vessels as metastatic cells. In yet another aspect, one device is placed upstream of the solid cancer tumor and a second device is placed downstream of the solid cancer tumor. This would effectively treat both the solid cancer tumor and any possible metastatic cells that have begun to spread in the patient.

In some embodiments, the patient is a mammal. In some embodiments, the patient is a human.

In some embodiments, the methods, devices and/or systems for treating cancer in a patient described herein, or the methods devices and/or systems for preventing or reducing cancer metastasis in a patient described herein, or the methods, devices and/or systems for treating a solid cancer tumor in a patient may include the following details, which may be combined with each other, unless clearly mutually exclusive.

Following lymphatic administration of an anti-PD-1 therapeutic agent or anti-PD-L1 therapeutic agent using the methods, devices and/or systems described herein, the anti-PD-1 or anti-PD-L1 may be taken up by initial lymphatics and delivered to one or more lymph nodes. For example, Examples 1 and 2 shows SOFUSA® is capable of delivering drug to LNs as shown through near intra-red imaging (NIRF) lymphatic imaging of ICG (indocyanine green, a fluorescent dye). By delivering an anti-PD-1 therapeutic agent or anti-PD-L1 therapeutic agent through the lymphatics, anti-PD-1 therapeutic agent or anti-PD-L1 therapeutic agent exposure to targets that reside within lymphatics are maximized for more effective anti-tumor responses.

In some embodiments, the device may be placed at one or more locations on the body of a patient, for example such as wrist, ankles, calf, or foot, among other positions on the body. The particular location on the body may be selected according to treatment need. In some embodiments, device placement and microneedle penetration may be optimized for infusions at the selected body part.

In some embodiments, lymphatic administration of an anti-PD-1 therapeutic agent or anti-PD-L1 therapeutic agent using the methods, devices and/or systems described herein may be associated with decreased pain experienced by the patient, as compared to intravenous administration (e.g., see Example 2). In some embodiments, pain may be reduced as compared to an equivalent amount of anti-PD-1 or anti-PD-L1 antibodies administered by intravenous, subcutaneous, intramuscular, intradermal or parenteral delivery routes.

In some embodiments, the lymphatic pumping rate may be increased following administration of anti-PD-1 or anti-PD-L1 antibodies using the methods, devices and/or systems described herein as compared to intradermal administration (e.g., see Example 2). In some embodiments, the lymphatic pumping rate may be between 0.1-5.0 pulses per minute. In some embodiments, the lymphatic pumping rate may be increased as compared to an equivalent amount of anti-PD-1 or anti-PD-L1 antibodies administered by intravenous, subcutaneous, intramuscular, intradermal or parenteral delivery routes. In some embodiments, the lymphatic pumping rate may be increased up to 1.2-fold, up to 1.6-fold, up to 1.8-fold, up to 2-fold, or up to 2.2-fold more as compared to an equivalent amount of anti-PD-1 or anti-PD-L1 antibodies administered by intravenous, subcutaneous, intramuscular, intradermal or parenteral delivery routes.

In some embodiments, the rate of serum concentration increase may be more gradual, and decreased following lymphatic administration of an anti-PD-1 therapeutic agent or anti-PD-L1 therapeutic agent using the methods, devices and/or systems described herein, as compared to rate of serum concentration increase following intravenous injection (e.g., see Examples 3 and 5). Accordingly, in some embodiments, a slope of serum concentration, e.g. ng/mL per hour, over a period of time, may be decreased following administration using the methods, devices and/or systems described herein, as compared to an equivalent amount of anti-PD-1 or anti-PD-L1 antibodies administered by intravenous, subcutaneous, intramuscular, intradermal or parenteral delivery routes. In some embodiments, the period of time may be up to, e.g. 5 minutes, 30 minutes, 1 hour, 2 hours, 5 hours, 12 hours, 24 hours, 48 hours or 72 hours. In some embodiments, serum concentration may increase more gradually as compared to an equivalent amount of anti-PD-1 or anti-PD-L1 antibodies administered by intravenous, subcutaneous, intramuscular, intradermal or parenteral delivery routes.

In some embodiments, following lymphatic administration of an anti-PD-1 therapeutic agent or anti-PD-L1 therapeutic agent using the methods, devices and/or systems described herein, bioavailability of the anti-PD-1 therapeutic agent or anti-PD-L1 therapeutic agent, may be up to, or up to about, 10%, 15% 20%, 25%, 30%, 35%, 40%, 45%, 50%, 55%, 60%, 65%, or 70%, for example such as 20% (e.g., see Example 3) or 35% (e.g., see Example 5), or 69% (e.g., see Example 7).

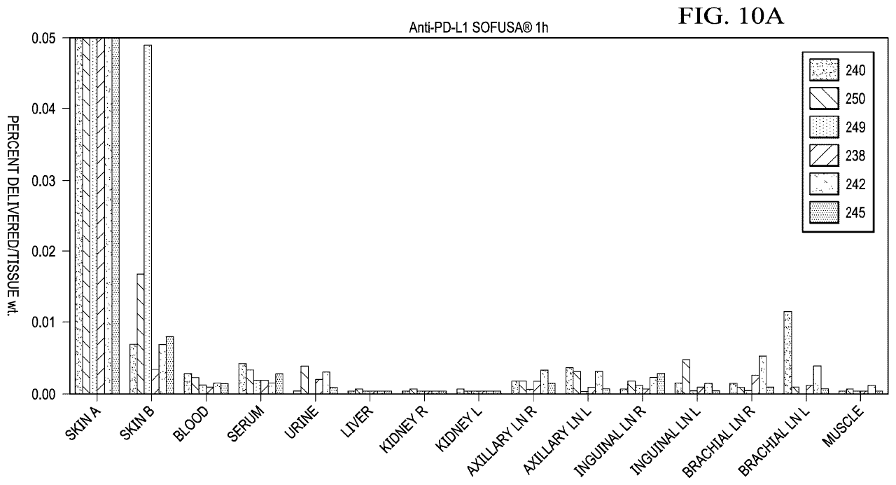

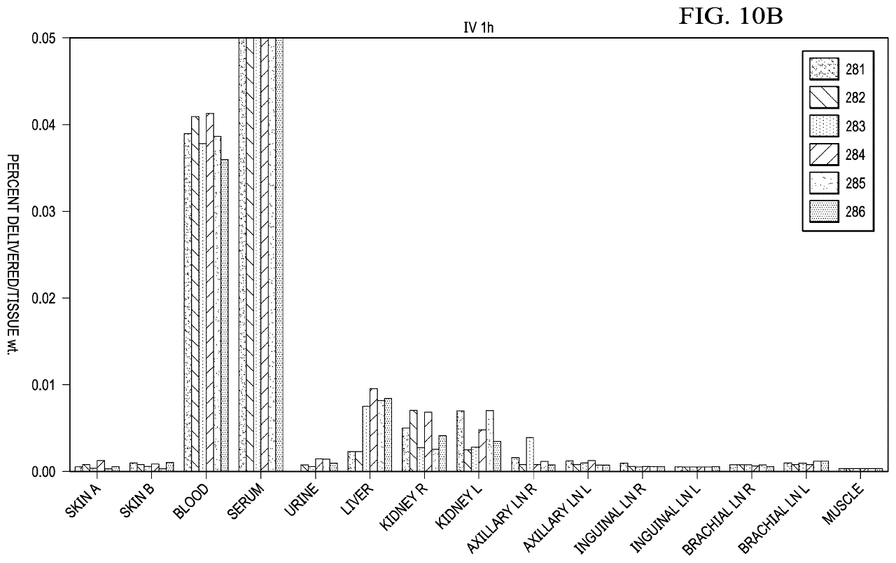

“Bioavailability” is measured herein as the area under the serum concentration versus time curve (AUC) over a period of time of an anti-PD-1 therapeutic agent or anti-PD-L1 therapeutic agent following lymphatic delivery divided by the dose, as compared to (AUC) over the period of time following intravenous administration of the anti-PD-1 therapeutic agent or anti-PD-L1 therapeutic agent, divided by the dose, assumed to deliver 100% of the anti-PD-1 therapeutic agent or anti-PD-L1 therapeutic agent into systemic circulation. In some embodiments, the period of time may be up to, e.g. 5 minutes, 30 minutes, 1 hour, 2 hours, 5 hours, 12 hours, 24 hours, 48 hours or 72 hours, or longer. For example, the period of time may be 672 hours (see, e.g., FIG. 7) or 72 hours (see, e.g., FIG. 8).

Without limitation to theory, the bioavailability may be correlated to the infusion time. Accordingly, in some embodiments, longer infusion durations may be used to increase bioavailability if necessary to achieve tumor inhibition.

In some embodiments, following lymphatic administration of an anti-PD-1 therapeutic agent or anti-PD-L1 therapeutic agent using the methods, devices and/or systems described herein, serum Tmax may be increased compared to intravenous administration. For example, in some embodiments, Tmax may be increased from about 5 minutes following intravenous administration to, or to about 10-100 hours, such as, or such as about, 10, 20, 30, 40, 50, 60, 70, 80, 90, or 100 hours, for example such as, or such as about, 24 hours (e.g., see Example 3) or 48 hours (e.g., see Examples 3 and 5) following lymphatic administration of an anti-PD-1 therapeutic agent or anti-PD-L1 therapeutic agent using the methods, devices and/or systems described herein.

In some embodiments, following lymphatic administration of an anti-PD-1 or anti-PD-L1 therapeutic agent using the methods, devices and/or systems described herein, serum Cmax may be decreased compared to intravenous administration. For example, in some embodiments, Cmax may be decreased about 2-fold following lymphatic administration as compared to intravenous administration (e.g., see Examples 3 and 5). In Example 3, Cmax was 85,000 ng/ml for intravenous compared to 31,000 ng/ml following lymphatic delivery. In some embodiments, serum Cmax may be decreased up to, or up to about, 1.5-fold, 2-fold, 2.5-fold, 3-fold, 3.5-fold, 4-fold, or more, as compared to an equivalent amount of anti-PD-1 or anti-PD-L1 antibodies administered by intravenous, subcutaneous, intramuscular, intradermal or parenteral delivery routes.

In some embodiments, following lymphatic administration of an anti-PD-1 or anti-PD-L1 therapeutic agent using the methods, devices and/or systems described herein, serum area under the curve AUC₀₋ₜ (e.g., in ng-hr/ml) over a period of time, e.g., denoted as time 0 to time t (0-t), may be decreased compared to intravenous administration. For example, in some embodiments, serum AUC₀₋ₜ may be decreased about 4-fold following administration of an anti-PD-1 or anti-PD-L1 therapeutic agent using the methods, devices and/or systems described herein, as compared to intravenous administration (e.g., see Examples 3 and 5). In Example 3, AUC₀₋₆₇₂ hours was 14,680,000 ng-hr/ml for intravenous administration compared to 3,414,000 ng-hr/ml for lymphatic delivery. In Example 5, AUC_{0-500 hours} was 41,300 ug/hr/ml for intravenous administration compared to 14,550 ug/hr/ml for lymphatic delivery. In some embodiments, serum AUC₀₋ₜ may be decreased up to, or up to about, 1.5-fold, 2-fold, 2.5-fold, 3-fold, 3.5-fold, 4-fold, or more, as compared to an equivalent amount of anti-PD-1 or anti-PD-L1 antibodies administered by intravenous, subcutaneous, intramuscular, intradermal or parenteral delivery routes.

PK may be measured using techniques known in the art, such as ELISA or using radiolabeled antibodies (e.g., see Example 3). Standard, highly accurate and precise methodologies for measuring blood serum concentration and therapeutic monitoring at desired time points may be used that are well known in the art, such as radioimmunoassays, high-performance liquid chromatography (HPLC), fluorescence polarization immunoassay (FPIA), enzyme immunoassay (EMIT) or enzyme-linked immunosorbant assays (ELISA). For calculating the absorption rate using the methods described above, the drug concentration at several time points may be measured starting immediately following administration and incrementally thereafter until a Cmax value is established and the associated absorption rate calculated.

In some embodiments, lymphatic administration of an anti-PD-1 or anti-PD-L1 therapeutic agent using the methods, devices and/or systems described herein, may result in increased delivery to LNs as compared to intravenous administration. For example, using the the methods, devices and/or systems described herein may result in about 2-fold more delivery to LNs, such as axillary, inguinal, and brachial lymph nodes compared to intravenous administration. (e.g., see Example 3). In some embodiments, delivery to LNs may be increased up to, or up to about, 1.5-fold, 2-fold, 2.5-fold, 3-fold, 3.5-fold, 4-fold, or more, as compared to an equivalent amount of anti-PD-1 or anti-PD-L1 antibodies administered by intravenous, subcutaneous, intramuscular, intradermal or parenteral delivery routes.

In some embodiments, for example, the lymphatic administration methods, devices and/or systems described herein may result in increased levels of anti-PD-1 or anti-PD-L1 in LNs, such as axillary, inguinal, and brachial lymph nodes, at early time points following administration, such as at the 1-hour timepoint, compared to IV. (e.g., see Examples 3 and 5). In some embodiments, the methods, devices and/or systems described herein may result in up to, or up to about, 120%, 140%, 160%, 180%, 200%, or more increased levels of anti-PD-1 or anti-PD-L1 in LNs at early time points following administration, such as at the 1-hour, 24-hour, or 72-hour timepoint, or at any intervening timepoint, as compared to an equivalent amount of anti-PD-1 or anti-PD-L1 antibodies administered by intravenous, subcutaneous, intramuscular, intradermal or parenteral delivery routes.

In some embodiments, for example, following lymphatic administration of an anti-PD-1 or anti-PD-L1 therapeutic agent using the methods, devices and/or systems described herein, lymph node levels may remain substantially or statistically constant over a period of time, e.g. over a period of up to 12 hours, 24 hours, 36 hours, 48 hours, 60 hours, 72 hours, or longer, whereas intravenous levels increase over the same period of time, such as up to 12 hours, 24 hours, 36 hours, 48 hours, 60 hours, 72 hours, or longer (e.g., see Examples 3 and 5).

In some embodiments, levels of therapeutic agent e.g. anti-PD-1 therapeutic agent or anti-PD-L1 therapeutic agent in systemic organs (e.g. liver and kidney) following lymphatic administration using the methods, devices and/or systems described herein may be decreased compared to intravenous administration for a period of time, such as up to 12 hours, 24 hours, 36 hours, 48 hours, 60 hours, 72 hours, or longer. (e.g., see Example 3). In some embodiments, levels of therapeutic agent e.g. anti-PD-1 therapeutic agent or anti-PD-L1 therapeutic agent in systemic organs (e.g. liver and kidney) following lymphatic administration using the methods, devices and/or systems described herein may be decreased from up to 10% to 75% as compared to intravenous administration. For example, in some embodiments, levels of therapeutic agent e.g. anti-PD-1 therapeutic agent or anti-PD-L1 therapeutic agent in one or more systemic organs (e.g. liver and kidney) following lymphatic administration using the methods, devices and/or systems described herein may be decreased up to 10%, 15% 20%, 25%, 30%, 35%, 40%, 45%, 50%, 55%, 60%, 65%, 70% or 75% as compared to levels of therapeutic agent e.g. anti-PD-1 therapeutic agent or anti-PD-L1 therapeutic agent in systemic organs (e.g. liver and kidney) as compared to an equivalent amount of anti-PD-1 or anti-PD-L1 antibodies administered by intravenous, subcutaneous, intramuscular, intradermal or parenteral delivery routes. Levels of anti-PD-1 therapeutic agent or anti-PD-L1 therapeutic agent in one or more systemic organs may be determined using any method known in the art identifiable by skilled persons upon reading the present disclosure. In some embodiments, levels of therapeutic agent e.g. anti-PD-1 therapeutic agent or anti-PD-L1 therapeutic agent in systemic organs (e.g. liver and kidney) may be decreased over a period of time, such as up to 12 hours, 24 hours, 36 hours, 48 hours, 60 hours, 72 hours, or longer, as compared to an equivalent amount of anti-PD-1 or anti-PD-L1 antibodies administered by intravenous, subcutaneous, intramuscular, intradermal or parenteral delivery routes.

In some embodiments, following lymphatic administration of a therapeutic agent e.g. anti-PD-1 therapeutic agent or anti-PD-L1 therapeutic agent using the methods, devices and/or systems described hererin, more than 90%, 95%, 99%, or 99.9% may be cleared by 28 days after administration. In contrast, for example, following intravenous administration, almost 83% was cleared in 28 days but the levels remained high and above 14 μg/mL (e.g., see Example 3).

In some embodiments, lymphatic administration of an anti-PD-1 or anti-PD-L1 therapeutic agent using the methods, devices and/or systems described herein provides improved efficacy of treatment of patients as compared to other administration routes, such as compared to an equivalent amount of anti-PD-1 or anti-PD-L1 antibodies administered by intravenous, subcutaneous, intramuscular, intradermal or parenteral delivery routes. Improved efficacy of treatment using administration of anti-PD-1 therapeutic agent or anti-PD-L1 therapeutic agent is expected, for example, based on improved efficacy in preclinical testing of a therapeutic anti-CTLA-4 antibody in a mouse model of cancer using SOFUSA® administration, as described in Sunkuk Kwon, Fred Christian Velasquez, John C. Rasmussen, Matthew R. Greives, Kelly D. Turner, John R. Morrow, Wen-Jen Hwu, Russell F. Ross, Songlin Zhang, and Eva M. Sevick-Muraca (2019), Nanotopography-based lymphatic delivery for improved anti-tumor responses to checkpoint blockade immunotherapy, Theranostics 9(26):8332-8343, 9(26): 8332-8343, 2019, doi:10.7150/thno.35280, the disclosure of which is incorporated herein by reference in its entirety.

In some embodiments, lymphatic administration of an anti-PD-1 or anti-PD-L1 therapeutic agent using the methods, devices and/or systems described herein may provide improved efficacy of anti-tumor response as compared to intravenous administration (e.g., see Prophetic Example 4). In some embodiments, administration of an anti-PD-1 or anti-PD-L1 therapeutic agent using the methods, devices and/or systems described herein provides improved efficacy of anti-tumor response as compared to an equivalent amount of anti-PD-1 or anti-PD-L1 antibodies administered by intravenous, subcutaneous, intramuscular, intradermal or parenteral delivery routes.

In some embodiments, following lymphatic administration of an anti-PD-1 or anti-PD-L1 therapeutic agent using the methods, devices and/or systems described herein, rate of tumor growth may be decreased as compared to intravenous administration. (e.g., see Prophetic Example 4). In some embodiments, rate of tumor growth may be decreased from an earlier time point compared to IV. In some embodiments, rate of tumor growth may be decreased, optionally from an earlier time point following administration, as compared to an equivalent amount of anti-PD-1 or anti-PD-L1 antibodies administered by intravenous, subcutaneous, intramuscular, intradermal or parenteral delivery routes

In some embodiments, lymphatic administration of an anti-PD-1 or anti-PD-L1 therapeutic agent using the methods, devices and/or systems described herein may provide a complete response to treatment. In some embodiments, the probability of a complete response to treatment may be increased following administration of an anti-PD-1 or anti-PD-L1 therapeutic agent using the methods, devices and/or systems described herein as compared to intravenous administration (e.g., see Prophetic Example 4). In some embodiments, the frequency or probability of a complete response following lymphatic administration of anti-PD-1 or anti-PD-L1 using the methods, devices and/or systems described herein may be at least, or at least about, 30%, 40%, 50%, 60%, 70%, 80%, 90%, 95%, or 99%. In some embodiments, the term “complete response” may refer to complete eradication of a tumor, such as a primary tumor, and/or a secondary tumor, or may refer to an undetectable tumor, such as an undetectable primary tumor or an undetectable secondary tumor. The term “secondary tumor” as used herein refers to a tumor in a different location to the primary tumor, as a result of metastasis of the primary tumor. In some embodiments, the probability of a complete response to treatment may be increased following administration of an anti-PD-1 or anti-PD-L1 therapeutic agent using the methods, devices and/or systems described herein as compared to an equivalent amount of anti-PD-1 or anti-PD-L1 antibodies administered by intravenous, subcutaneous, intramuscular, intradermal or parenteral delivery routes.

In some embodiments, lymphatic administration of an anti-PD-1 or anti-PD-L1 therapeutic agent using the methods, devices and/or systems described herein may allow the use of lower therapeutically effective doses of the anti-PD-1 or anti-PD-L1 therapeutic agent compared to therapeutically effective doses of the anti-PD-1 or anti-PD-L1 therapeutic agent administered by an intravenous, subcutaneous, intramuscular, intradermal or parenteral delivery route.

In some embodiments, lymphatic administration of an anti-PD-1 or anti-PD-L1 therapeutic agent using the methods, devices and/or systems described herein may result in tumor growth inhibition that is increased by up to 10%, 20%, 30%, 40%, 50%, 60%, 70%, 80%, 90% or 100%, or up to 2-fold, 3-fold, 4-fold, 5-fold, 6-fold, 7-fold, 8-fold, 9-fold, 10-fold, 20-fold, 30-fold, 40-fold, or 50-fold compared to tumor growth inhibition following an equivalent amount of the anti-PD-1 therapeutic agent or the anti-PD-L1 therapeutic agent administered by an intravenous, subcutaneous, intramuscular, intradermal or parenteral delivery route (e.g., see Example 8).

In some embodiments, lymphatic administration of an anti-PD-1 or anti-PD-L1 therapeutic agent using the methods, devices and/or systems described herein may result in tumor growth inhibition that is equal to or better than tumor growth inhibition following up to 1.5-fold, 2-fold, 2.5-fold, 3-fold, 3.5-fold, 4-fold, 4.5-fold, or 5-fold greater amount of the anti-PD-1 therapeutic agent or the anti-PD-L1 therapeutic agent administered by an intravenous, subcutaneous, intramuscular, intradermal or parenteral delivery route (e.g., see Example 8).

In some embodiments, lymphatic administration of an anti-PD-1 or anti-PD-L1 therapeutic agent using the methods, devices and/or systems described herein may result in decreased metastasis as compared to intravenous administration. (e.g., see Prophetic Example 4). In some embodiments, lymphatic administration of an anti-PD-1 or anti-PD-L1 therapeutic agent using the methods, devices and/or systems described herein may result in up to 20%, 25%, 30%, 35%, 40%, 45%, 50%, 55%, 60%, 65%, 70% or 75% decrease in metastasis, such as a decrease in number, size, or probability of formation of secondary tumors, as compared to an equivalent amount of anti-PD-1 or anti-PD-L1 antibodies administered by intravenous, subcutaneous, intramuscular, intradermal or parenteral delivery routes.

In some embodiments, lymphatic administration of an anti-PD-1 or anti-PD-L1 therapeutic agent using the methods, devices and/or systems described herein may result in a greater exposure of anti-PD-1 or anti-PD-L1 to T-cells in TDLNs as compared to following intravenous administration. In some embodiments, lymphatic administration of an anti-PD-1 or anti-PD-L1 therapeutic agent using the methods, devices and/or systems described herein may result in a greater exposure of anti-PD-1 or anti-PD-L1 to T-cells in TDLNs, e.g. an increase of up to 1.5-fold, 2-fold, 2.5-fold, or more, as compared to an equivalent amount of anti-PD-1 or anti-PD-L1 antibodies administered by intravenous, subcutaneous, intramuscular, intradermal or parenteral delivery routes.

In some embodiments, lymphatic administration of an anti-PD-1 or anti-PD-L1 therapeutic agent using the methods, devices and/or systems described herein may result in a greater exposure of anti-PD-1 or anti-PD-L1 to tumor cells in a lymphatic system as compared to following intravenous administration. In some embodiments, lymphatic administration of an anti-PD-1 or anti-PD-L1 therapeutic agent using the methods, devices and/or systems described herein may result in a greater exposure of anti-PD-1 or anti-PD-L1 to tumor cells in the lymphatic system, e.g. an increase of up to 1.5-fold, 2-fold, 2.5-fold, or more, as compared to an equivalent amount of anti-PD-1 or anti-PD-L1 antibodies administered by intravenous, subcutaneous, intramuscular, intradermal or parenteral delivery routes.

In some embodiments, lymphatic administration of an anti-PD-1 or anti-PD-L1 therapeutic agent using the methods, devices and/or systems described herein may result in a greater number of tumor-infiltrating lymphocytes (TILs) as compared to intravenous administration (e.g., see Prophetic Example 4). In some embodiments, lymphatic administration of an anti-PD-1 or anti-PD-L1 therapeutic agent using the methods, devices and/or systems described herein may result in a greater number of TILs, e.g. an increase of up to 1.5-fold, 2-fold, 2.5-fold, or more, as compared to an equivalent amount of anti-PD-1 or anti-PD-L1 antibodies administered by intravenous, subcutaneous, intramuscular, intradermal or parenteral delivery routes.

In some embodiments, lymphatic administration of an anti-PD-1 or anti-PD-L1 therapeutic agent using the methods, devices and/or systems described herein may result in decreased toxicity compared with intravenous administration (e.g., see Example 5). In some embodiments, toxicity may include hematological toxicity, such as decreased platelets. In some embodiments, lymphatic administration of an anti-PD-1 or anti-PD-L1 therapeutic agent using the methods, devices and/or systems described herein may result in decreased toxicity, such as one or more parameters of hematological toxicity as compared to an equivalent amount of anti-PD-1 or anti-PD-L1 antibodies administered by intravenous, subcutaneous, intramuscular, intradermal or parenteral delivery routes. For example, in some embodiments, intravenous administration of an anti-PD-1 or anti-PD-L1 therapeutic agent may result in up to a 90% reduction in platelet levels, as compared to platelet levels following lymphatic administration of an anti-PD-1 or anti-PD-L1 therapeutic agent using the methods, devices and/or systems described herein (see Example 5).

In some embodiments, administration of an anti-PD-1 or anti-PD-L1 therapeutic agent using the methods, devices and/or systems described herein may result in more consistent, less variable, serum levels of anti-PD-1 or anti-PD-L1 as compared with intravenous administration (e.g., see Example 5). In some embodiments, administration of an anti-PD-1 or anti-PD-L1 therapeutic agent using the methods, devices and/or systems described herein may result in less variable serum levels of anti-PD-1 or anti-PD-L1 as compared to an equivalent amount of anti-PD-1 or anti-PD-L1 antibodies administered by intravenous, subcutaneous, intramuscular, intradermal or parenteral delivery routes.

In some embodiments, administration of an anti-PD-1 or anti-PD-L1 therapeutic agent using the methods, devices and/or systems described herein may result in reduced systemic exposure and maximize delivery to the tumor and to tumor draining LNs where tumor antigens are present (e.g., see prophetic Example 6).

In some embodiments, lymphatic administration of an anti-PD-1 or anti-PD-L1 therapeutic agent using the methods, devices and/or systems described herein may result in reduced incidence or severity of one or more Adverse Events (AE's) as compared to intravenous administration (e.g., see prophetic Example 6), or as compared to an equivalent amount of anti-PD-1 or anti-PD-L1 antibodies administered by intravenous, subcutaneous, intramuscular, intradermal or parenteral delivery routes.

The term Adverse Event or AE as used herein refers to any noxious, unintended, or untoward medical occurrence that may appear or worsen in a patient following administration. It may be a new intercurrent illness, a worsening concomitant illness, an injury, or any concomitant impairment of the participant's health, including laboratory test values, regardless of etiology. Any worsening (e.g., any clinically significant adverse change in the frequency or intensity of a pre-existing condition) may be considered an AE.

In general, immunotherapies as a group have off-target effects and toxicities common to them. Some of these include interstitial pneumonitis, colitis, skin reactions, low levels of platelets and white blood cells, inflammation of the brain or spinal cord, neuromuscular adverse events including myositis, Guillain-Barré syndrome, myasthenia gravis; myocarditis and cardiac insufficiency, acute adrenal insufficiency, and nephritis, among others.

For example, in some embodiments, the AE may be fatigue, musculoskeletal pain, decreased appetite, pruritus, diarrhea, nausea, rash, pyrexia, cough, dyspnea, constipation, pain, and abdominal pain (e.g., see KEYTRUDA® (pembrolizumab) package insert). Merck & Co., Inc., Whitehouse Station, NJ; 2019).

In some embodiments, the AE may be an immune-related Adverse Event or irAE. The term immune-related Adverse Event or irAE as used herein refers to toxicities associated with checkpoint inhibitors that are autoimmune or autoinflammatory in origin. The toxicities may differ in their severity, grade, and tolerability. Immune-related adverse events may occur at any part of the body, and may include, for example, interstitial pneumonia, colitis, hypothyroidism, liver dysfunction, skin rash, vitiligo, hypophysitis, type 1 diabetes, renal dysfunction, myasthenia gravis, neuropathy, myositis, among others.

In some embodiments, the irAE may include cytokine release syndrome (CRS) or cytokine storm (CS). The term cytokine storm as used herein refers to a form of systemic inflammatory response syndrome that may arise as a complication of some monoclonal antibody drugs. Symptoms of CRS or CS may include fever, fatigue, loss of appetite, muscle and joint pain, nausea, vomiting, diarrhea, rashes, fast breathing, rapid heartbeat, low blood pressure, seizures, headache, confusion, delirium, hallucinations, tremor, and loss of coordination. In addition, infusion-related reactions may including hypersensitivity and anaphylaxis, and other infusion-related reactions including rigors, chills, wheezing, pruritus, flushing, rash, hypotension, hypoxemia, and fever.

In some embodiments, administration of an anti-PD-1 or anti-PD-L1 therapeutic agent using the methods, devices and/or systems described herein may result in improved pharmacodynamic effects as compared with an equivalent amount of anti-PD-1 or anti-PD-L1 antibodies administered by intravenous, subcutaneous, intramuscular, intradermal or parenteral delivery routes, such as improved levels of T-cell exhaustion markers (e.g., PD-1, Lag-3, Tim-3, and ICOS in malignant CD4+ and tumor-infiltrating CD8 T-cells in tumor tissue); detection of pembrolizumab in tumor tissue; and Ki67 expression in tumor tissue (e.g., see prophetic Example 6).

In some embodiments, administration of an anti-PD-1 or anti-PD-L1 therapeutic agent using the methods, devices and/or systems described herein may result in improvements in Ki67 expression in blood; receptor occupancy of anti-PD-1 or anti-PD-L1 in blood, as compared an equivalent amount of anti-PD-1 or anti-PD-L1 antibodies administered by intravenous, subcutaneous, intramuscular, intradermal or parenteral delivery routes (e.g., see prophetic Example 6).

In some embodiments, administration of an anti-PD-1 or anti-PD-L1 therapeutic agent using the methods, devices and/or systems described herein may result in improved efficacy of treatment as compared to an equivalent amount of anti-PD-1 or anti-PD-L1 antibodies administered by intravenous, subcutaneous, intramuscular, intradermal or parenteral delivery routes. For example, as described in prophetic Example 6, for assessment of efficacy of treating CTCL, efficacy may be assessed using methods known in the art, such as (1) Modified Severity Weighted Assessment Tool (mSWAT) for response in skin, (2) Composite Assessment of Index Lesion Severity for response in skin, (3) Flow cytometry for Sezary cell count for response in blood, (4) PET/CT scan for participants with stage IB disease with >30% skin involvement, stage IIB-IVB disease, Sezary Syndrome (SS), or transformed Mycosis Fungoides (MF). Scans not needed for stage IB participants or participants with <30% skin involvement, and (5) Global Response Score for assessment of response, among others.

Pharmacokinetic and biodistribution studies using the device described herein, e.g. the SOFUSA® platform, show improved effects as compared to other administration routes, including the following. SOFUSA® has higher LN/Blood concentration during first 30 hours (e.g., with a wear/administration time of 1 hour). Biodistribution results demonstrate sustained higher lymphatic concentrations with lower systemic exposure in other organ systems. SOFUSA® delivery of to the lymph system and lymph nodes is direct delivery that starts immediately upon initiation of the SOFUSA® infusion. These results may be obtained with 50% of the dose of IV. The biodistribution profile of SOFUSA® is consistent with a differentiated safety and efficacy profile versus intravenous administration (e.g. higher immune system concentrations and target exposure with lower systemic concentrations, reduced dosing, reduced Adverse Events (AE's), such as immune-related AE's, due to lower systemic concentrations and higher lymphatic concentrations, and location of treatment accessing tumor draining lymph nodes.

In some embodiments, the delivery of the anti-PD-1 therapeutic agent or anti-PD-L1 therapeutic agent to the lymphatic system as described herein may be combined with administration of the anti-PD-1 therapeutic agent or anti-PD-L1 therapeutic agent via one or more additional routes of administration, such as one or more of intravenous, subcutaneous, intramuscular, intradermal or parenteral delivery routes.

Pre-clinical and clinical studies show SOFUSA® nanotopography-draped microneedles result in significantly higher rates of absorption with both small and large molecules as compared to undraped microneedles. Preclinical data suggest enhanced absorption with the SOFUSA® device as compared to subcutaneous injections resulting in higher bioavailability as compared to subcutaneous injection. Biodistribution results as compared to IV, subcutaneous, or intradermal injections demonstrate sustained higher lymphatic concentrations with lower systemic exposure in other organ systems. Radiolabeling data indicates direct delivery to the lymph nodes starts immediately upon initiation and was sustained up to 36 hours after the device was removed. Improved effects of SOFUSA® administration include efficacy and safety, including lack of bolus, lower systemic concentrations, and lower dose. SOFUSA® biodistribution profile show the potential for differentiated safety and efficacy profiles compared to other administration routes such as intravenous and subcutaneous administration, due to higher lymphatic system concentrations and lower systemic concentrations.

Devices that comprise an array of microneedles suitable for use herein are known in the art. Particular exemplary structures and devices comprising a means for controllably delivering one or more agents to a patient are described in International Patent Application Publication Nos. WO 2014/188343, WO 2014/132239, WO 2014/132240, WO 2013/061208, WO 2012/046149, WO 2011/135531, WO 2011/135530, WO 2011/135533, WO 2014/132240, WO 2015/16821, and International Patent Applications PCT/US2015/028154 (published as WO 2015/168214 A1), PCT/US2015/028150 (published as WO 2015/168210 A1), PCT/US2015/028158 (published as WO 2015/168215 A1), PCT/US2015/028162 (published as WO 2015/168217 A1), PCT/US2015/028164 (published as WO 2015/168219 A1), PCT/US2015/038231 (published as WO 2016/003856 A1), PCT/US2015/038232 (published as WO 2016/003857 A1), PCT/US2016/043623 (published as WO 2017/019526 A1), PCT/US2016/043656 (published as WO 2017/019535 A1), PCT/US2017/027879 (published as WO 2017/189258 A1), PCT/US2017/027891 (published as WO 2017/189259 A1), PCT/US2017/064604 (published as WO 2018/111607 A1), PCT/US2017/064609 (published as WO 2018/111609 A1), PCT/US2017/064614 (published as WO 2018/111611 A1), PCT/US2017/064642 (published as WO 2018/111616 A1), PCT/US2017/064657 (published as WO 2018/111620 A1), and PCT/US2017/064668 (published as WO 2018/111621 A1), all of which are incorporated by reference herein in their entirety.

In some aspects of the embodiments described herein, the one or more therapeutic agents are administered by applying one or more devices to one or more sites of the skin of the patient. One nonlimiting example of a device comprising a plurality of microneedles that is suitable for use with all of the methods disclosed herein is the SOFUSA® drug delivery platform (Sorrento Therapeutics, Inc.).

In some embodiments, the device is placed in direct contact with the skin of the patient. In some embodiments, an intervening layer or structure will be between the skin of the patient and the device. For example, surgical tape or gauze may be used to reduce possible skin irritation between the device and the skin of the patient. When the microneedles extend from the apparatus, they will contact and, in some instances, penetrate the epidermis or dermis of the patient in order to deliver the therapeutic agent to the patient. The delivery of the therapeutic agent can be to the circulatory system, the lymphatic system, the interstitium, subcutaneous, intramuscular, intradermal or a combination thereof. In some embodiments, the therapeutic agent is delivered directly to the lymphatic system of the patient. In some aspects, the therapeutic agent is delivered to the superficial vessels of the lymphatic system.

The term “proximate” as used herein is intended to encompass placement on and/or near a desired therapeutic target. Placement of the device proximate to the therapeutic target results in the administered therapeutic agent entering the lymphatic system and traversing to the intended therapeutic target. Additionally, placement of the device may be such that the administered therapeutic agent is directly administered to the therapeutic target.

In some embodiments described herein, the methods comprising a device comprising a plurality of microneedles may comprise delivering one or more agents through a device comprising two or more delivery structures that are capable of penetrating the stratum corneum of the skin of a patient and obtaining a delivery depth and volume in the skin and controllably delivering one or more agents at the administration rates as described herein. The delivery structures may be attached to a backing substrate of the device and arranged at one or a plurality of different angles for penetrating the stratum corneum and delivering the one or more agents. In some aspects, described herein the backing substrate comprising the delivery structures may be in contact with the skin of a patient and may have a cylindrical, rectangular, or geometrically irregular shape. The backing substrate further comprises a two dimensional surface area that in some aspects may be from about 1 mm² to about 10,000 mm². In some aspects, the delivery structures may comprise any geometric shape (e.g., a cylindrical, rectangular or geometrically irregular shape). In addition, the delivery structures may comprise a length and cross sectional surface area. In some aspects, the delivery structures may have an overall length that is greater than a cross sectional diameter or width. In some other aspects, the delivery structures may have a cross sectional diameter or width greater than an overall length. In some aspects, the cross sectional width of each of the delivery structures may be from about 5 μm to about 140 μm and the cross sectional area may be from about 25 μm² to about 65,000 μm², including each integer within the specified range. In some embodiments, the length of each of the delivery structures may be from about 10 μm to about 5,000 μm, from about 50 to about 3,000 μm, from about 100 to about 1,500 μm, from about 150 to about 1,000 μm, from about 200 to about 800 μm, from about 250 to about 750 μm, or from about 300 to about 600 μm. In some aspects, the length of each of the delivery structures may be from about 10 μm to about 1,000 μm, including each integer within the specified range. The surface area and cross-sectional surface areas as described herein may be determined using standard geometric calculations known in the art.

The delivery structures described herein need not be identical to one another. A device having a plurality of delivery structures may each have various lengths, outer diameters, inner diameters, cross-sectional shapes, nanotopography surfaces, and/or spacing between each of the delivery structures. For example, the delivery structures may be spaced apart in a uniform manner, such as, for example, in a rectangular or square grid or in concentric circles. The spacing may depend on numerous factors, including height and width of the delivery structures, as well as the amount and type of an agent that is intended to be delivered through the delivery structures. In some aspects, the spacing between each delivery structure may be from about 1 μm to about 1500 μm, including each integer within the specified range. In some aspects, the spacing between each deliver structure may be about 200 μm, about 300 μm, about 400 μm, about 500 μm, about 600 μm, about 700 μm, about 800 μm, about 900 μm, about 1000 μm, about 1100 μm, about 1200 μm, about 1300 μm, about 1400 μm or about 1500 μm. About as used in this context, “about” means±50 m.

In some embodiments described herein, the device may comprise a needle array in the form of a patch. In some aspects, the array of needles are able to penetrate a most superficial layer of the stratum corneum and initially deliver one or more agents as described herein to at least a portion or all of the non-viable epidermis, at least a portion of or all of the viable epidermis, and/or at least a portion of the viable dermis of a subject and subsequently to the lymphatic system of the patient. These needles may further comprise nanotopography on the surface of the needle in a random or organized pattern. In some aspects, the nanotopography pattern may demonstrate fractal geometry.

In some embodiments, the delivery structures may comprise an array of needles in fluid connection with a liquid carrier vehicle comprising one or more agents, such as the anti-PD-1 or anti-PD-L1 therapeutic agents described herein. In some aspects, the needles are microneedles. In some aspects, the array of needles may comprise between 2 and 50,000 needles with structural means for controlling skin penetration and fluid delivery to the skin (e.g., penetrating and delivering to the skin), see e.g., International Patent Application PCT/US2017/064668 (published as WO 2018/111621 A1), which is incorporated by reference herein in its entirety. In some other aspects, the array of needles may further comprise a manufactured random or structured nanotopography on each needle. The needle or needle array may be comprised in a system, such as a system wherein the device is attached to additional components of a therapeutic agent delivery apparatus comprising components such as fluidic delivery rate controls, adhesives for attaching to the skin, fluidic pumps, and the like. If desired, the rate of delivery of the agent may be variably controlled by the pressure-generating means. Desired delivery rates as described herein to the epidermis may be initiated by driving the one or more agents described herein with the application of pressure or other driving means, including pumps, syringes, pens, elastomer membranes, gas pressure, piezoelectric, electromotive, electromagnetic or osmotic pumping, or use of rate control membranes or combinations thereof.

In some embodiments described herein, devices comprising a plurality of microneedles as described herein functions as a permeability enhancer and may increase the delivery of one or more agents through the epidermis. This delivery may occur through modulating transcellular transport mechanisms (e.g., active or passive mechanisms) or through paracellular permeation. Without being bound by any theory, the nanostructured or nanotopography surface may increase the permeability of one or more layers of the viable epidermis, including the epidermal basement membrane by modifying cell/cell tight junctions allowing for paracellular or modifying cellular active transport pathways (e.g., transcellular transport) allowing for diffusion or movement and/or active transport of an administered agent through the viable epidermis and into the underlying viable dermis. This effect may be due to modulation of gene expression of the cell/cell tight junction proteins. As previously mentioned, tight junctions are found within the viable skin and in particular the viable epidermis. The opening of the tight junctions may provide a paracellular route for improved delivery of any agent, such as those that have previously been blocked from delivery through the skin.

Interaction between individual cells and structures of the nanotopography may increase the permeability of an epithelial tissue (e.g., the epidermis) and induce the passage of an agent through a barrier cell and encourage transcellular transport. For instance, interaction with keratinocytes of the viable epidermis may encourage the partitioning of an agent into the keratinocytes (e.g., transcellular transport), followed by diffusion through the cells and across the lipid bilayer again. In addition, interaction of the nanotopography structure and the corneocytes of the stratum corneum may induce changes within the barrier lipids or corneodesmosomes resulting in diffusion of the agent through the stratum corneum into the underlying viable epidermal layers. While an agent may cross a barrier according to paracellular and transcellular routes, the predominant transport path may vary depending upon the nature of the agent.

In some embodiments described herein, the device may interact with one or more components of the epithelial tissue to increase porosity of the tissue making it susceptible to paracellular and/or transcellular transport mechanisms. Epithelial tissue is one of the primary tissue types of the body. Epithelial tissues that may be rendered more porous may include both simple and stratified epithelium, including both keratinized epithelium and transitional epithelium. In addition, epithelial tissue encompassed herein may include any cell types of an epithelial layer including, without limitation, keratinocytes, endothelial cells, lymphatic endothelial cells, squamous cells, columnar cells, cuboidal cells and pseudostratified cells. Any method for measuring porosity may be used including, but not limited to, any epithelial permeability assay. For example, a whole mount permeability assay may be used to measure epithelial (e.g., skin) porosity or barrier function in vivo see, for example, Indra and Leid., Methods Mol Biol. (763) 73-81, which is incorporated by reference herein for its teachings thereof.

In some embodiments described herein, the structural changes induced by the presence of a nanotopography surface on a barrier cell are temporary and reversible. It was surprisingly found that using nanostructured nanotopography surfaces results in a temporary and completely reversible increase in the porosity of epithelial tissues by changing junctional stability and dynamics, which, without being bound by any theory, may result in a temporary increase in the paracellular and transcellular transport of an administered agent through the epidermis and into the viable dermis. Thus, in some aspects, the increase in permeability of the epidermis or an epithelial tissue elicited by the nanotopography, such as promotion of paracellular or transcellular diffusion or movement of one or more agents, returns to a normal physiological state that was present before contacting the epithelial tissue with a nanotopography following the removal of the nanotopography. In this way, the normal barrier function of the barrier cell(s) (e.g., epidermal cell(s)) is restored and no further diffusion or movement of molecules occurs beyond the normal physiological diffusion or movement of molecules within the tissue of a subject.

These reversible structural changes induced by the nanotopography may function to limit secondary skin infections, absorption of harmful toxins, and limit irritation of the dermis. Also, the progressive reversal of epidermal permeability from the top layer of the epidermis to the basal layer may promote the downward movement of one or more agents through the epidermis and into the dermis and prevent back flow or back diffusion of the one or more agents back into the epidermis.

In some embodiments described herein, are methods for applying a device having a plurality of microneedles to the surface of the skin a subject for the treatment of a disease or disorder described herein. In some aspects, the device is applied to an area of the subject's skin, wherein the location of the skin on the body is dense in lymphatic capillaries and/or blood capillaries. Multiple devices may be applied to one or more locations of the skin having a dense network of lymphatic capillaries. In some aspects, 1, 2, 3, 4, 5, or more devices may be applied. These devices may be applied spatially separate or in close proximity or juxtaposed with one another. Exemplary and non-limiting locations dense with lymphatics comprise the palmar surfaces of the hands, the scrotum, the plantar surfaces of the feet and the lower abdomen. The location of the device will be selected based on the medical condition of the patient and the assessment of a medical professional.

In some embodiments described herein, at least a portion of or all of the therapeutic agent may be directly delivered or administered to an initial depth in the skin comprising the nonviable epidermis and/or the viable epidermis. In some aspects, a portion of therapeutic agent may also be directly delivered to the viable dermis in addition to the epidermis. The range of delivery depth will depend on the medical condition being treated and the skin physiology of a given patient. This initial depth of delivery may be defined as a location within the skin, wherein a therapeutic agent first comes into contact as described herein. Without being bound by any theory, it is thought that the administered agent may move (e.g., diffuse) from the initial site of delivery (e.g., the non-viable epidermis, the viable epidermis, the viable dermis, or the interstitium) to a deeper position within the viable skin. For example, a portion of or all of an administered agent may be delivered to the non-viable epidermis and then continue to move (e.g., diffuse) into the viable epidermis and past the basal layer of the viable epidermis and enter into the viable dermis. Alternatively, a portion of or all of an administered agent may be delivered to the viable epidermis (i.e., immediately below the stratum corneum) and then continue to move (e.g., diffuse) past the basal layer of the viable epidermis and enter into the viable dermis. Lastly, a portion of or all of an administered agent may be delivered to the viable dermis. The movement of the one or more active agents throughout the skin is multifactorial and, for example, depends on the liquid carrier composition (e.g., viscosity thereof), rate of administration, delivery structures, etc. This movement through the epidermis and into the dermis may be further defined as a transport phenomenon and quantified by mass transfer rate(s) and/or fluid mechanics (e.g., mass flow rate(s)).

Thus, in some embodiments described herein, the therapeutic agent may be delivered to a depth in the epidermis wherein the therapeutic agent moves past the basal layer of the viable epidermis and into the viable dermis. In some aspects described herein, the therapeutic agent is then absorbed by one or more susceptible lymphatic capillary plexus then delivered to one or more lymph nodes and/or lymph vessels.

In some embodiments, the device comprises a fluid delivery apparatus, wherein the fluid delivery apparatus comprises: a fluid distribution assembly wherein a cap assembly is coupled to a cartridge assembly, and the cartridge assembly is slidably coupled to a plenum assembly, and a mechanical controller assembly is slidably coupled to the cartridge assembly; a collet assembly constituting the housing of the fluid delivery apparatus and being slidably coupled to the fluid distribution assembly; and a plurality of microneedles fluidically connected with the fluid distribution assembly having a surface comprising nanotopography, the plurality of microneedles being capable of penetrating the stratum corneum of the skin of a patient and controllably delivering the anti-PD-1 therapeutic agent or the anti-PD-L1 therapeutic agent to a depth below the surface of the skin.

In some embodiments, the device delivers the anti-PD-1 therapeutic agent or the anti-PD-L1 therapeutic agent to a depth below the surface of the skin of from about 50 μm to about 4000 μm, from about 250 μm to about 2000 μm, or from about 350 μm to about 1000 μm. In some embodiments, each of the microneedles in the device has a length between about 200 to about 800 μm, between about 250 to about 750 μm, or between about 300 to about 600 μm.

In some embodiments described herein, the distribution of depths in the skin, wherein a portion of the one or more agents is initially delivered, which results in uptake of the one or more therapeutic agents by one or more susceptible tumors or inflammatory locus, or by lymph vessels that feed into the tumors or inflammatory locus, ranges from about 5 μm to about 4,500 μm. Because the thickness of the skin can vary from patient to patient based on numerous factors, including, but not limited to, medical condition, diet, gender, age, body mass index, and body part, the required depth to deliver the therapeutic agent will vary. In some aspects, the delivery depth is from about 50 μm to about 4000 μm, from about 100 to about 3500 μm, from about 150 μm to about 3000 μm, from about 200 μm to about 3000 μm, from about 250 μm to about 2000 μm, from about 300 μm to about 1500 μm, or from about 350 μm to about 1000 μm. In some aspects, the delivery depth is about 50 μm, about 100 μm, about 150 μm, about 200 μm, about 250 μm, about 300 μm, about 350 μm, about 400 μm, about 450 μm, about 500 μm, about 600 μm, about 700 μm, about 800 μm, about 900 μm, or about 1000 μm. As used in this context, “about” means±50 μm.

In some embodiments described herein, the therapeutic agent may be delivered in a liquid carrier solution. In one aspect, the tonicity of the liquid carrier may be hypertonic to the fluids within the blood capillaries or lymphatic capillaries. In another aspect, the tonicity of a liquid carrier solution may be hypotonic to the fluids within the blood capillaries or lymphatic capillaries. In another aspect, the tonicity of a liquid carrier solution may be isotonic to the fluids within the blood capillaries or lymphatic capillaries. The liquid carrier solution may further comprise at least one or more pharmaceutically acceptable excipients, diluent, cosolvent, particulates, or colloids. Pharmaceutically acceptable excipients for use in liquid carrier solutions are known, see, for example, Pharmaceutics: Basic Principles and Application to Pharmacy Practice (Alekha Dash et al. eds., 1st ed. 2013), which is incorporated by reference herein for its teachings thereof.

In some embodiments described herein, the therapeutic agent is present in a liquid carrier as a substantially dissolved solution, a suspension, or a colloidal suspension. Any suitable liquid carrier solution may be utilized that meets at least the United States Pharmacopeia (USP) specifications, and the tonicity of such solutions may be modified as is known, see, for example, Remington: The Science and Practice of Pharmacy (Lloyd V. Allen Jr. ed., 22nd ed. 2012. Exemplary non-limiting liquid carrier solutions may be aqueous, semi-aqueous, or nonaqueous depending on the bioactive agent(s) being administered. For example, an aqueous liquid carrier may comprise water and any one of or a combination of a water-miscible vehicles, ethyl alcohol, liquid (low molecular weight) polyethylene glycol, and the like. Non-aqueous carriers may comprise a fixed oil, such as corn oil, cottonseed oil, peanut oil, or sesame oil, and the like. Suitable liquid carrier solutions may further comprise any one of a preservative, antioxidant, complexation enhancing agent, a buffering agent, an acidifying agent, saline, an electrolyte, a viscosity enhancing agent, a viscosity reducing agent, an alkalizing agent, an antimicrobial agent, an antifungal agent, a solubility enhancing agent or a combination thereof.

In some embodiments described herein, the therapeutic agent is delivered to the viable skin, wherein the distribution of depths in the viable skin for delivery of the agent is immediately past the stratum corneum of the epidermis but above the subcutaneous tissue, which results in uptake of the agent by the lymphatic vasculature of the patient. In some aspects, the depth in the viable skin for delivering one or more agents ranges from about 1 μm to about 4,500 μm beyond the stratum corneum, but still within the viable skin above the subcutaneous tissue.

Non-limiting tests for assessing initial delivery depth in the skin may be invasive (e.g., a biopsy) or non-invasive (e.g., imaging). Conventional non-invasive optical methodologies may be used to assess delivery depth of an agent into the skin including remittance spectroscopy, fluorescence spectroscopy, photothermal spectroscopy, or optical coherence tomography (OCT).

Imaging using methods may be conducted in real-time to assess the initial delivery depths. Alternatively, invasive skin biopsies may be taken immediately after administration of an agent, followed by standard histological and staining methodologies to determine delivery depth of an agent. For examples of optical imaging methods useful for determining skin penetration depth of administered agents, see, Sennhenn et al., Skin Pharmacol. 6(2) 152-160 (1993), Gotter et al., Skin Pharmacol. Physiol. 21 156-165 (2008), or Mogensen et al., Semin. Cutan. Med. Surg 28 196-202 (2009), each of which are incorporated by reference herein for their teachings thereof.

In some embodiments described herein are methods for the extended delivery (or administration) of the therapeutic agent as described herein. The device comprising a plurality of microneedles is configured such that that the flow rate of the therapeutic agent from the device into the patient can be adjusted. As such, the length of time required will vary accordingly. In some aspects, the flow rate of the device is adjusted such that the therapeutic agent is administered over from about 0.5 hours to about 72 hours. In some aspects the time period for administration is about 1 hour, 2 hours, 3 hours, 4 hours, 5 hours, 6 hours, 7 hours, 8 hours, 9 hours, 10 hours, 12 hours, 15 hours 18 hours, 21 hours, 24 hours, 27 hours, 30 hours, 33 hours, 36 hours, 39 hours, 42 hours, 45 hours, 48 hours, 51 hours, 54 hours, 57 hours, 60 hours, 63 hours, 66 hours, 69 hours or 72 hours. In other aspects, the time period for administration is selected based on the medical condition of the patient and an assessment by the medical professional treating the patient.

In some embodiments described herein, one or more agents in a liquid carrier solution are administered to an initial approximate volume of space below the outer surface of the skin. The one or more therapeutic agents in a liquid carrier solution initially delivered to the skin (e.g., prior to any subsequent movement or diffusion) may be distributed within, or encompassed by an approximate three dimensional volume of the skin. The one or more initially delivered agents exhibits a Gaussian distribution of delivery depths and will also have a Gaussian distribution within a three dimensional volume of the skin tissue.

In some embodiments described herein, the flow rate of the therapeutic agent to the skin per single microneedle as described herein may be about 0.01 μl per hour to about 500 μl per hour. In some aspects, the flow rate for each individual microneedle is from about 0.1 μl per hour to about 450 μl per hour, about 0.5 μl per hour to about 400 μl per hour, about 1.0 μl per hour to about 350 μl per hour, about 5.0 μl per hour to about 300 μl per hour, about 5.0 μl per hour to about 250 μl per hour, about 10 μl per hour to about 200 μl per hour, about 15 μl per hour to about 100 μl per hour, or about 20 μl per hour to about 50 μl per hour. In some aspects, the flow rate for each individual microneedle is about 1 μl per hour, 2 μl per hour, 5 μl per hour, 10 μl per hour, 15 μl per hour, 20 μl per hour, 25 μl per hour, 30 μl per hour, 40 μl per hour, 50 μl per hour, 75 μl per hour, or 100 μl per hour. Each individual microneedle will have a flow rate that contributes to the overall device flow rated. The maximum overall flow rate will be flow rate of each individual microneedle multiplied by the total number of microneedles. The overall controlled flow rate of all of the combined microneedles may be from about 0.2 μl per hour to about 50,000 μl per hour. The device is configured such that that the flow rate can be controlled appropriately. The flow rate will be based upon the medical condition of the patient and an assessment by the medical professional treating the patient.

The disclosures of the specifications, claims and drawings of the following non-limiting and non-exhaustive list of patent applications and patents related to devices, in addition to other patent applications and patents referenced elsewhere in this disclosure, including lymphatic delivery devices, methods of providing the same, and methods of using the same for lymphatic administration, are incorporated by reference in their entireties: U.S. Pat. Nos. 9,962,536 and 9,550,053, U.S. application Ser. Nos. 15/305,193, 15/305,206, 15/305,201, 15/744,346, 14/354,223, and International Patent Application No.'s PCT/US2017/027879, PCT/US2017/027891, PCT/US2016/043656, PCT/US2017/064604, PCT/US2017/064609, PCT/US2017/064642, PCT/US2017/064614, PCT/US2017/064657, PCT/US2017/064668, and U.S. Provisional Patent Application No. 62/678,601, filed May 31, 2018, U.S. Provisional Patent Application No. 62/678,592, filed May 31, 2018, and U.S. Provisional Patent Application No. 62/678,584, filed May 31, 2018, and International Application No. PCT/US2019/034736.

Informal Sequence Listing

| | Heavy chain variable domain region | Light chain variable domain region |
|---|---|---|
| GA1 | QVQLQQSGPGLVRPSQTLSLSCDISGDS | LPVLTQPASVSGSPGQSITISCTG |
| | VSSNSATWNWIRQSPSRGLEWLGRTF | TSSDVGGYNYVSWYQQHPGKA |
| | YRSKWYHDYALSVKSRITINPDTSKNQ | PKLMIYEVSKRPSGVPDRFSGSK |
| | FSLQLNSVSPGDTAVYFCVREDIDGRL | SGNTASLTVSGLQAEDEADYYC |
| | DYWGQGTLVTVSS SEQ ID NO. 1 | SAWDDSLNADVFGGGTKLTVL |
| | | SEQ ID NO. 2 |
| GA2 | QVQLVQSGSELKKPGASVKISCKASGY | QPVLTQPPSASGTPGQRVTISCSG |
| | IFSDNGVNWVRQAPGQGLEWMGWIN | SSSNIGTNTVNWYQQVPGTAPK |
| | TKIGNPTYAQGFTGRFVFSLDTSVSTTY | LLIHGNDQRPSGVPDRFSGSKSD |
| | LQISSLQAGDTAVYYCAREHDYYYGM | TSASLAITGLQSDDDADYYCSA |
| | DVWGQGTTVTVSS SEQ ID NO. 3 | WDDSLNADVFGGGTKLTVL |
| | | SEQ ID NO. 4 |
| GB1 | MAEVQLLESGAEVKKPGASVKVSCKA | QAVLTQPPSASATPGQRVTISCS |
| | SGYTFTSHYMHWVRQAPGQGLEWMG | GSDSNIGTNYVYWYQQFPGTAP |
| | VINPSGGSTSYAQKFQGRVTMTRDTST | QPLIYRDNQRPSGVPDRFSGSKS |
| | STVYMDLSSLRSEDTAVYYCARRSEA | GTSASLAISGLRSEDEATYFCST |
| | YYHGMDVWGQGTTVTVSS SEQ ID | WDDSLNGWVFGGGTKLTVL |
| | NO. 5 | SEQ ID NO. 6 |
| GB6 | EVQLVQSGSELKKPGASVKISCKASGY | QPVLTQPRSVSGSPGQSITTSCTG |
| | IFSDNGVNWVRQAPGQGLEWMGWIN | TSSDVGGYNYVSWYQQHPGKA |
| | TKIGNPTYAQGFTGRFVFSLDTSISTTY | PKLMIYEVSNRPSGVSNRFSGSK |
| | LQISSLQAGDTAVYYCAREHDYYYGM | SGNTASLTISGLQAEDEADYYCS |
| | DVWGQGTTVTVSS SEQ ID NO. 7 | SYTSSSTLEVFGTGTKVTVL SEQ |
| | | ID NO. 8 |
| GH1 | QVQLVESGGGLVQPGGSLRLSCEATGF | SYELMQPPSVSGAPGQRVTISCT |
| | TFSRYWMHWVRQAPGKGLEWVSSISS | GSSSNIGAAYDVHWYQQLPGKA |
| | SSSYIYYADSVKGRFTISRDNAKNSLYL | PKLVMFANSNRPSGVPDRFSGSK |
| | QMNSLRAEDTAVYYCARDTLEYYGSG | SGTSASLAITGLQAEDEADYYCQ |
| | ILENAMGYYGMDVWGQGTTVTVSS | SYDISLRAYVFGTGTKLTVL SEQ |
| | SEQ ID NO. 9 | ID NO. 10 |
| A2 | EVQLVESGGGLVRPGGSLRLACAASGF | SYELMQPPSASGTPGQRVTISCS |
| | SFSDYYMTWIRQAPGRGLEWIAYISDS | GSSSNIGTNTVNWYQHLPGTAP |
| | GQTVHYADSVKGRFTISRDNTKNSLFL | KLLIYSNNQRPSGVPDRFSGSKS |
| | QVNTLRAEDTAVYYCAREDLLGYYLQ | GTSASLAISGLQSEDEADYYCAT |
| | SWGQGTLVTVSS SEQ ID NO. 11 | WDDSPNGWVFGGGTKLTVL |
| | | SEQ ID NO. 12 |
| C7 | EVQLVESGGGVVQPGRSLRLSCAASGF | QAVLTQPPSVSAAPGQRVTISCS |
| | TFSSYWMSWVRQAPGKGLEWVANIK | GSNSNIADTYVSWYQQLPGTAP |
| | QDGSEKYYVDSVKGRFTISRDNAKNSL | RLLIYDNDQRPSGIPDRFSGSKSG |
| | YLQMNSLRAEDTAVYYCAREGEHDAF | TSATLGITGLQTGDEADYYCGT |
| | DIWGQGTMVTVSS SEQ ID NO. 13 | WDSSLSGVFGTGTKVTVL SEQ |
| | | ID NO. 14 |
| H7 | QVQLVQSGSELKKPGASVKISCKASGY | QSVLTQPASVSGSPGQSVTISCTG |
| | IFSDNGVNWVRQAPGQGLEWMGWIN | SSSDVGAYNFVSWYRQYPGKAP |
| | TKIGNPTYAQGFTGRFVFSLDTSVSTTY | KLLIYEVNKRPSDVPDRFSGSKF |
| | LQISSLQAGDTAVYYCAREHDYYYGM | GNTASLTVSGLQADDEADYYCS |
| | DVWGQGTTVTVSS SEQ ID NO. 15 | SYAGSTDVFGTGTKVTVL SEQ |
| | | ID NO. 16 |
| SH-A4 | QVQLVQSGSELKKPGASVKISCKASGY | LPVLTQPPSVSGTPGQRVTISCSG |
| | IFSDNGVNWVRQAPGQGLEWMGWIN | SSSNIGSNTVNWYQQLPGTAPKL |
| | TKIGNPTYAQGFTGRFVFSLDTSVSTTY | LIYTNNQRPSGVPDRFSGSKSGT |
| | LQISSLQAGDTAVYYCAREHDYYYGM | SASLAISGLQSEDEADYYCAAW |
| | DVWGQGTTVTVSS SEQ ID NO. 17 | DESLNGDVFGTGTKVTVL SEQ |
| | | ID NO. 18 |
| SH-A9 | QVQLVQSGAEVKKPGASVKVSCKASG | AIRMTQSPSFLSASVGDRVTITCR |
| | YTFTRYTMHWVRQAPGQRLEWMGWI | TSQNIYNYLNWYQQKPGKAPEL |
| | NAGNGNTKYSQKFQGRVTITRDTSAST | LIFVASSLQSGVPSRFSGSGSGTD |
| | AYMELSSLRSEDTAVYYCAKVSAGTE | FTLTISSLQPEDFATYFCLQDHSY |
| | SWFDPWGQGTLVTVSS SEQ ID NO. 19 | PYTFGQGTKVEIK SEQ ID NO. 20 |
| RG1B3 | QVQLVQSGAEVKKPGASVKVSCKASG | LPVLTQPPSVSEVPGQRVTISCSG |
| | YTFTSYGISWVRQAPGQGLEWMGWIS | GISNIGSNAVNWYQHFPGKAPKL |
| | AYNGNTNYAQKLQGRVTMTTDTSTST | LIYYNDLLPSGVSDRFSASKSGTS |
| | AYMELRSLRSDDTAVYYCARGLYGDE | ASLAISGLRSEDEADYYCAAWD |
| | DYWGQGTLVTVSS SEQ ID NO. 21 | DNLSAYVFATGTKVTVL SEQ ID |
| | | NO. 22 |
| RG1H10 | QVQLVQSGSELKKPGASVKISCKASGY | QSALTQPPSASGSPGQSVTISCTG |
| | IFSDNGVNWVRQAPGQGLEWMGWIN | TSSDVGGYNYVSWYQHHPGKA |
| | TKIGNPTYAQGFTGRFVFSLDTSISTTY | PKLMIYEVSKRPSGVPDRFSGSK |
| | LQISSLQAGDTAVYYCAREHDYYYGM | SAITASLTISGLLTEDEADYYCSA |
| | DVWGQGTTVTVSS SEQ ID NO. 23 | WDDSLNADVFGGGTKVTVL |
| | | SEQ ID NO. 24 |
| RG1H11 | QMQLVQSGAEVKKPGESLKISCKGSG | DIQMTQSPSSLSASVGDRVTITCR |
| | YSFTSYWIGWVRQMPGKGLEWMGIIY | ASQSISTYLNWYQQKPGKAPKV |
| | PGDSDTRYSPSFQGQVTISADKSISTAY | LITDASSLETGVPSRFSGSGSGTD |
| | LQWSSLKASDTAMYYCASGVTRKRYS | FTFTISSLQPEDTATYFCQQYDDL |
| | SSWPPFDYWGQGTLVTVSS SEQ ID | PPTFGQGTKLEIK SEQ ID NO. 26 |
| | NO. 25 | |
| RG2H7 | QVQLQQWGAGLLKSSETLSLSCAVYG | QAGLTQPRSVSGSPGQSVTISCT |
| | GTFRDDHWSWIRQPPGKGLEWIGESH | GTSSDVGGYNYVSWYQQHPGK |
| | HTGRTIYNPSLRSRVTMSIDTSKNEFSLI | APKLMIYDVTKRPSGVSNRFSGS |
| | LRSVTAADTATYFCARGNNYVWGNQ | KSGNTASLTISGLQAEDEADYYC |
| | EDFWGQGTLVTVSS SEQ ID NO. 27 | SSYTSSSTYVFGTGTKVTVL SEQ |
| | | ID NO. 28 |
| RG2H10 | QVQLQQSGPGLVRPSQTLSLSCDISGDS | QPVLTQPASVSGSPGQSITISCTG |
| | VSSNSATWNWIRQSPSRGLEWLGRTF | TSSDVGGYNYVSWYQQHPGKA |
| | YRSKWYHDYALSVKSRITINPDTSKNQ | PKLMIYDVSNRPSGVSNRFSGSK |
| | FSLQLNSVSPGDTAVYFCVREDIDGRL | SGNTASLAITGLQSDDDADYYCS |
| | DYWGQGTLVTVSS SEQ ID NO. 29 | AWDDSLNADVFGGGTKLTVL |
| | | SEQ ID NO. 30 |
| RG3E12 | EVQLVESGGALVQPGGSLRLSCAVSGF | QAGLTQPPSVSKGLRQTATLTCT |
| | TFSDHYMDWVRQAPGKGLEWVARSR | GNSNNIGDQGAAWLQQHQGHPP |
| | NKGNSYTTEYAASVRGRFTISRDDSKN | RLLSYRNNNRPSGISERLSASRSG |
| | SLYLQMNSLKTEDTAVYYCVRVGVVP | NIASLTITGLQPEDEADYYCSAW |
| | ALDGMDVWGQGTTVTVSS SEQ ID | DSSLSVWVFGGGTKLTVL SEQ |
| | NO. 31 | ID NO. 32 |
| RG4A6 | EVQLVESGGGLVQPGGSLRLSCAASGF | QAVLTQPPSASGTPGQRVTISCS |
| | TFSSYWMHWVRQAPGKGLEWVSSISS | GSSSNIGSNTVNWYQQLPGTAPK |
| | SSSYIYYADSVKGRFTISRDNAKNSLYL | LLIYSNNQRPSGVPDRFSGSKSG |
| | QMNSLRAEDTAVYYCARDTLEYYGSG | TSASLAISGLQSEDEADYYCAA |
| | ILENAMGYYGMDVWGQGTTVTVSS | WDDSLNGYVFGTGTKLTVL |
| | SEQ ID NO. 33 | SEQ ID NO. 34 |
| RG5D9 | EVQLLESGGGVVQTGRSLRLSCSDSGS | AIRMTQSPSTLSASVGDRVTITCR |
| | TFRSQAMHWVRQTPGKGLEWLAVTS | ASENIRNLLAWYQQKPGKAPEL |
| | HDGSKTYYADSVKGRFTISRDNSKNTL | LIHGASTLGTGVPSRFSGGGSGT |
| | YLQMNSLRGEDTAVYYCARGGRGYT | EFTLTISSLQPDDFATYYCQQYES |
| | YDHSFFDYWGQGTLVTVSS SEQ ID | YFNTFGQGTKVEIK SEQ ID NO. |
| | NO. 35 | 36 |
| RG1H10- | QVQLVQSGSELKKPGASVKISCKASGY | QSALTQPPSASGSPGQSVTISCTG |
| H2A-22-1S | IFSDNGVNWVRQAPGQGLEWMGWIN | TSSDVGGYNYVSWYQHHPGKA |
| | TKDGNPTYAQGFTGRFVFSLDTSISTTY | PKLMIYEVSKRPSGVPDRFSGSK |
| | LQISSLQAGDTAVYYCAREHDYYYGM | SAITASLTISGLLTEDEADYYCSA |
| | DVWGQGTTVTVSS SEQ ID NO. 37 | WDDSLNADVFGGGTKVTVL |
| | | SEQ ID NO. 24 |
| RG1H10- | QVQLVQSGSELKKPGASVKISCKASGY | QSALTQPPSASGSPGQSVTISCTG |
| H2A-27-2S | IFSDNGVNWVRQAPGQGLEWMGWIN | TSSDVGGYNYVSWYQHHPGKA |
| | TKTGNPTYAQGFTGRFVFSLDTSISTTY | PKLMIYEVSKRPSGVPDRFSGSK |
| | LQISSLQAGDTAVYYCAREHDYYYGM | SAITASLTISGLLTEDEADYYCSA |
| | DVWGQGTTVTVSS SEQ ID NO. 38 | WDDSLNADVFGGGTKVTVL |
| | | SEQ ID NO. 24 |
| RG1H10- | QVQLVQSGSELKKPGASVKISCKASGY | QSALTQPPSASGSPGQSVTISCTG |
| 3C | TFSDNGVNWVRQAPGQGLEWMGWIN | TSSDVGGYNYVSWYQHHPGKA |
| | TKDGNPTYAQGFTGRFVFSLDTSISTTY | PKLMIYEVSKRPSGVPDRFSGSK |
| | LQISSLQAGDTAVYYCAREHDYYYGM | SAITASLTISGLLTEDEADYYCSA |
| | DVWGQGTTVTVSS SEQ ID NO. 39 | WDDSLNADVFGGGTKVTVL |
| | | SEQ ID NO. 24 |
| RG1H10- | QVQLVQSGSELKKPGASVKISCKASGY | QSALTQPPSASGSPGQSVTISCTG |
| 16C | TFSDNGVNWVRQAPGQGLEWMGWIN | TSSDVGGYNYVSWYQHHPGKA |
| | TKTGNPTYAQGFTGRFVFSLDTSISTTY | PKLMIYEVSKRPSGVPDRFSGSK |
| | LQISSLQAGDTAVYYCAREHDYYYGM | SAITASLTISGLLTEDEADYYCSA |
| | DVWGQGTTVTVSS SEQ ID NO. 40 | WDDSLNADVFGGGTKVTVL |
| | | SEQ ID NO. 24 |
| RG1H10- | QVQLVQSGSELKKPGASVKISCKASGY | QSALTQPPSASGSPGQSVTISCTG |
| 17C | KFSDNGVNWVRQAPGQGLEWMGWIN | TSSDVGGYNYVSWYQHHPGKA |
| | TKSGNPTYAQGFTGRFVFSLDTSISTTY | PKLMIYEVSKRPSGVPDRFSGSK |
| | LQISSLQAGDTAVYYCAREHDYYYGM | SAITASLTISGLLTEDEADYYCSA |
| | DVWGQGTTVTVSS SEQ ID NO. 41 | WDDSLNADVFGGGTKVTVL |
| | | SEQ ID NO. 24 |
| RG1H10- | QVQLVQSGSELKKPGASVKISCKASGY | QSALTQPPSASGSPGQSVTISCTG |
| 19C | KFSDNGVNWVRQAPGQGLEWMGWIN | TSSDVGGYNYVSWYQHHPGKA |
| | TKTGNPTYAQGFTGRFVFSLDTSISTTY | PKLMIYEVSKRPSGVPDRFSGSK |
| | LQISSLQAGDTAVYYCAREHDYYYGM | SAITASLTISGLLTEDEADYYCSA |
| | DVWGQGTTVTVSS SEQ ID NO. 42 | WDDSLNADVFGGGTKVTVL |
| | | SEQ ID NO. 24 |
| RG1H10- | QVQLVQSGSELKKPGASVKISCKASGY | QSALTQPPSASGSPGQSVTISCTG |
| 21C | TFSDNGVNWVRQAPGQGLEWMGWIN | TSSDVGGYNYVSWYQHHPGKA |
| | TKSGNPTYAQGFTGRFVFSLDTSISTTY | PKLMIYEVSKRPSGVPDRFSGSK |
| | LQISSLQAGDTAVYYCAREHDYYYGM | SAITASLTISGLLTEDEADYYCSA |
| | DVWGQGTTVTVSS SEQ ID NO. 43 | WDDSLNADVFGGGTKVTVL |
| | | SEQ ID NO. 24 |
| RG1H10- | QVQLVQSGSELKKPGASVKISCKASGY | QSALTQPPSASGSPGQSVTISCTG |
| 23C2 | KFSDNGVNWVRQAPGQGLEWMGWIN | TSSDVGGYNYVSWYQHHPGKA |
| | TKDGNPTYAQGFTGRFVFSLDTSISTTY | PKLMIYEVSKRPSGVPDRFSGSK |
| | LQISSLQAGDTAVYYCAREHDYYYGM | SAITASLTISGLLTEDEADYYCSA |
| | DVWGQGTTVTVSS SEQ ID NO. 44 | WDDSLNADVFGGGTKVTVL |
| | | SEQ ID NO. 24 |

### Examples

The following examples are provided for illustrative purposes only. They are not intended to and should not be interpreted as disclosing the entire breadth of the invention of the present disclosure. Furthermore, although the Examples include specific details, their teaching are applicable to and combinable with other details of the disclosure from other examples or other portions of this specification unless such combinations are clearly mutually exclusive.

### Example 1. SOFUSA® Lymphatic Infusion Device Administration of ICG Dye to LNs in Mice in Preclinical Studies

SOFUSA® DoseConnect™ is a microneedle drug delivery device with a nanotopographical imprinted polyether, ether, ketone film heat-formed over each microneedle on the array (FIG. 1; FIG. 2). The nanotopographical film-microneedle combination has been found to increase permeability through the skin epidermis layer by remodeling tight junction proteins initiated via integrin binding to the nanotopography.

Twenty-four hours prior to SOFUSA® DoseConnect™ administration, mice were anesthetized with isofluorane and the dorsal region was shaved and covered with depilatory cream (Nair Sensitive) for 8 minutes. The cream was then wiped off with warm, wet paper towels, followed by alcohol wipes. SOFUSA® DoseConnect™ was then applied to the dorsal region using a plastic shell with a skin adhesive. A hand-held applicator was then placed over the plastic shell to insert the microneedles into the skin. The operation of the device was as follows. The applicator strikes the microneedles with a post traveling at a velocity of 6 m/s. There is a total of 100 microneedles over the area of 66 mm². With the microneedles inserted in the skin, the syringe pump was started to deliver indocyanine green (ICG).

50 μL of 0.5 mg/mL ICG was infused over an hour on the right dorso-lateral side of isoflurane anesthetized healthy mice. Lymphatic imaging was performed using non-invasive near-infrared fluorescence (NIRF) imaging as described in Sevick-Muraca E M, Kwon S, Rasmussen J C. Emerging lymphatic imaging technologies for mouse and man. J Clin Invest. 2014; 124:905-14.

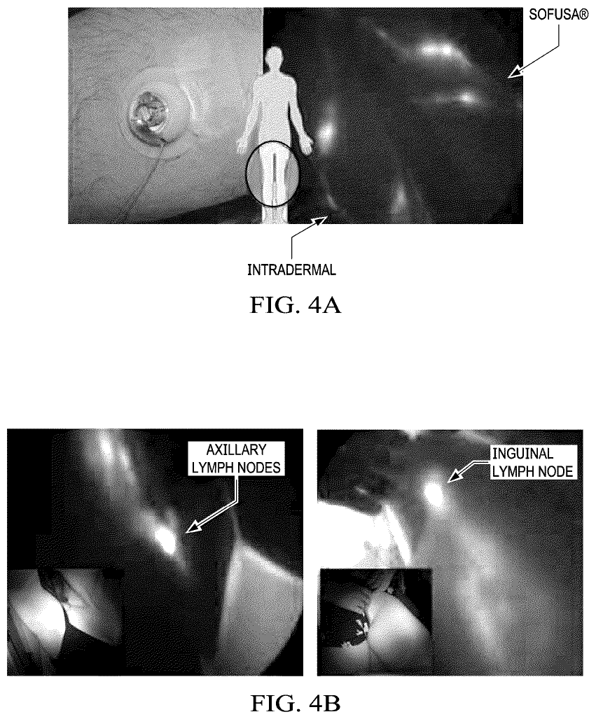

NIRF imaging showed that SOFUSA® can effectively infuse 100 μL/h of ICG into the epidermal spaces wherein the initial lymphatics take up the agent enabling visualization of the propulsion of ICG-laden lymph into the brachial LNs (FIG. 3).

### Example 2. SOFUSA® Lymphatic Infusion Device Administration of ICG Dye in Clinical Studies: Near-Infrared Fluorescence Imaging of ICG Shows Lymphatically Directed Delivery to Draining LNs

Before SOFUSA® technology can be considered for infusion of checkpoint blockade immunotherapies in cancer patients, its feasibility for lymphatic delivery needs to be assessed in human subjects. In a pilot study of 12 human volunteers, this Example shows SOFUSA® is capable of delivering drug to LNs as shown through NIRF lymphatic imaging of ICG.

In a pilot study of 12 normal humans, 0.25 mg/mL solution of indocyanine green (ICG) was infused lymphatically for a period of 60 min using a calibrated infusion pump (Model 4100, Atlanta BioMedical Corporation), and the nanotopographical device positioned on the dorsal aspect of feet, lateral aspect of ankles, medial aspect of calf, and/or the wrist. The uptake of ICG was monitored using a custom built near-infrared fluorescence imaging system employing a Gen III GaAs intensifier coupled to a sCMOS (Zhu B, Rasmussen J C, Litorja M, Sevick-Muraca E M. Determining the Performance of Fluorescence Molecular Imaging Devices Using Traceable Working Standards With SI Units of Radiance. IEEE Trans Med Imaging. 2016; 35:802-11) to visualize delivery to inguinal and axillary LNs, and to quantify lymphatic propulsion and transport of ICG-laden lymph at contralateral sites after intradermal injection. Optimization of device placement and microneedle tissue penetration was performed on the first 8 subjects. In the last four subjects, infusion rates were varied between 0.2-1 mL/h and afterwards, lymphatic propulsion was analyzed from acquired images by counting the number of ICG-laden lymphatic “packets” that crossed a chosen anatomical landmark. Lymphatic pumping rates resulting from infusion were compared to those from the contralateral locations where ICG was administered intradermally. Contralateral intradermal (i.d.) injections were made using a conventional insulin syringe and 31 gauge needle to deliver 0.1 mL of 0.25 mg/mL ICG solution and were often made following desensitization with cold spray for those volunteers who were sensitive to needle prick. Cold spray was not used for application of the SOFUSA® infusion device and pain was assessed via a visual analog scale (VAS) questionnaire for each infusion device applied. Up to five different devices were placed on each volunteer for simultaneous infusion.

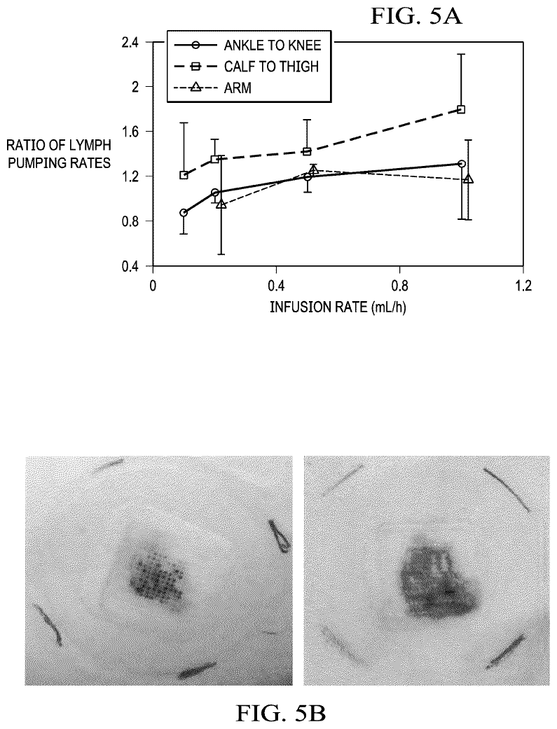

In the first 8 subjects, device placement and microneedle penetration were optimized for infusions on the dorsal aspect of the wrist, lateral aspect of ankles, and medial aspect of calf and could be achieved with less success on the dorsal foot. In subjects of low BMI, the placement on the foot and wrist were often complicated with incomplete penetration of microneedles into the dermis as visualized by ICG leakage and impaired uptake. FIG. 4A shows the lymphatic vessels imaged following SOFUSA® infusions and contralateral i.d. injections in the medial aspect of the calf and the expected symmetry of functional lymphatic vessels that pump ICG-laden lymph to the regional axillary and inguinal LNs (FIG. 4B). With subjects sitting upright, it was found that lymphatic pumping rates expectedly ranged between 0.4-3.3 pulses/min, consistent with past work (Tan I C, Maus E A, Rasmussen J C, Marshall M V, Adams K E, Fife C E, et al. Assessment of lymphatic contractile function after manual lymphatic drainage using near-infrared fluorescence imaging. Arch Phys Med Rehabil. 2011; 92:756-64 el).

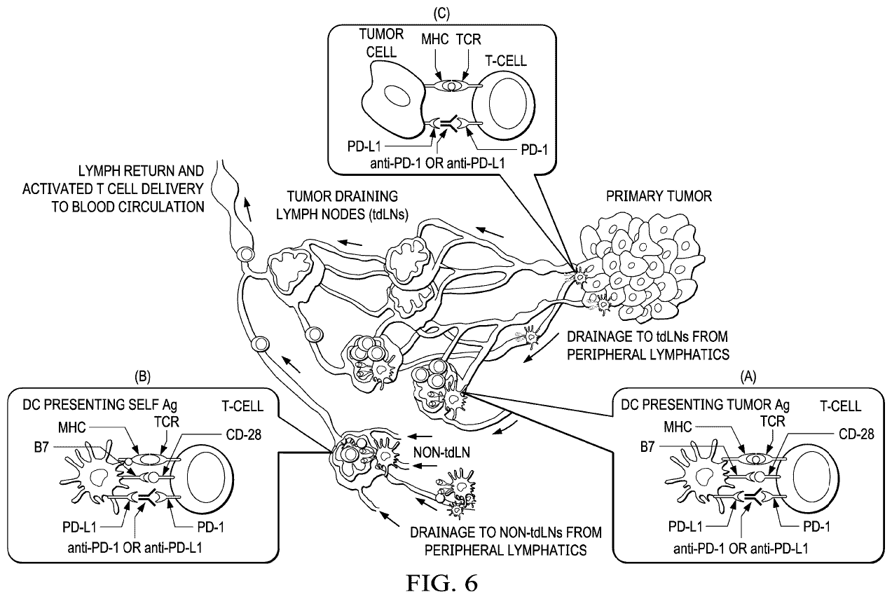

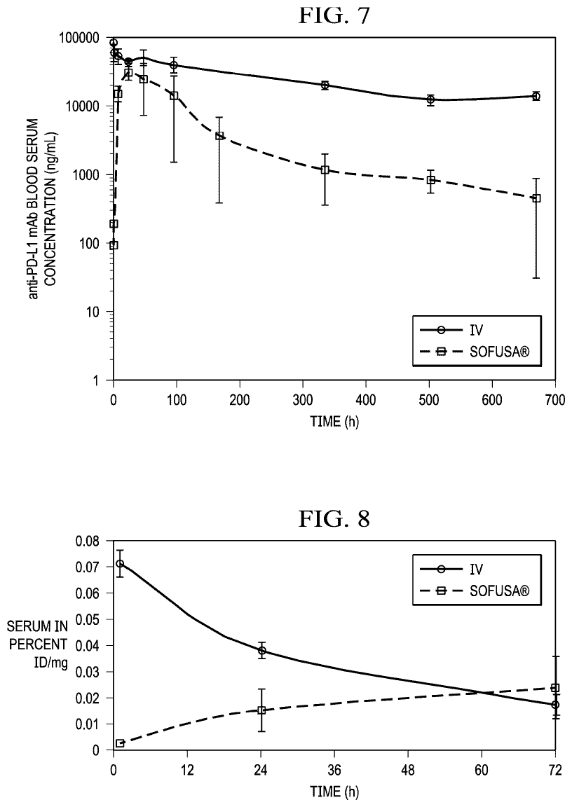

The lymphatic pumping rates of ICG-laden lymph resulting from SOFUSA® infusion were consistently faster when ICG was infused at rates >0.2 mL/h with SOFUSA® than when delivered via i.d. injection at the contralateral site. In addition, as shown in FIG. 5A, the ratio of lymphatic pumping rates resulting from SOFUSA® infusion to that resulting from i.d. administration also tended to increase with SOFUSA® infusion rates, but, due to the small sample size, no further analysis or statistical significance could be determined. Further investigation of whether the nanotopographic features on the microneedle array are responsible for the enhanced filling and increased active transport remain will be performed. Interestingly, previous data in animals showed the nanostructured microneedle array devices cumulatively delivered significantly more etanercept than microneedle array devices without nanostructured coating by measuring the appearance of drug in the serum of rats and rabbits (Walsh L, Ryu J, Bock S, Koval M, Mauro T, Ross R, et al. Nanotopography facilitates in vivo lymphatic delivery of high molecular weight therapeutics through an integrin-dependent mechanism. Nano lett. 2015; 15:2434-41). At SOFUSA® infusion rates of 1 mL/hr, ICG “pooling” at the infusion site was visualized after removal of the device, indicating that the lymphatic uptake in the initial lymphatics in the sub-epidermal space was slower than the infusion rate. FIG. 5B shows the transient demarcations of ICG left by the microneedle array immediately after removal. After 24 hours, these demarcations disappear.

Subjective assessment of pain owing to application of SOFUSA®, infusion, and removal of SOFUSA® device was performed for each device application using a visual analog scale (VAS) questionnaire with a range of 0-100 with the value of 0 associated with no discomfort sensation and with the value of 100 associated with extreme pain. The average±SD VAS pain score for application, infusion, and removal of the SOFUSA® was 8±9, 5±8, and 1±4, respectively, indicating that the device caused between no pain to mild pain (Jensen M P, Chen C, Brugger A M. Interpretation of visual analog scale ratings and change scores: a reanalysis of two clinical trials of postoperative pain. J Pain. 2003; 4:407-14). There were no adverse events associated with the ICG or the SOFUSA® device during the time of the study or at study follow-up, which occurred 24 hours after the study.

### Example 3. Pharmacokinetics and Biodistribution Results of Anti-PD-L1 mAb in Healthy Mice

This Example describes pharmacokinetics and biodistribution of SOFUSA® DoseConnect™ intra-lymphatic delivery of an anti-PD-L1 mAb in healthy mice versus intravenous delivery.

It is expected that the results described in this Example in relation to an anti-PD-L1 antibody will be equally applicable to an anti-PD-1 antibody.

It is now well established that anti-PD-1 or anti-PD-L1 monoclonal antibodies (mAb) that block the interaction between PD-1 on T cells and PD-L1 on tumor cells can boost T cell activity and proliferation, leading to enhanced antitumor immunity and durable remissions in a proportion of patients. However, up to 70% of patients or more do not respond to these therapeutic agents (www.immuneoncia.com). It is expected that responsiveness may be increased by using an improved intra-lymphatic drug delivery route that would enable anti-PD-L1 mAB or anti-PD-1 mAb to access the immune system more directly and increase antitumor immunity.

The purpose of this study was to assess whether increased intra-lymphatic delivery from the SOFUSA® DoseConnect™ hollow microneedle device could improve the biodistribution of an anti-PD-L1 mAb in the lymphatic system versus systemic administration. In addition, this Example compares the pharmacokinetics of anti-PD-L1 mAb between SOFUSA® DoseConnect™ intra-lymphatic delivery versus systemic administration. The animal model was a healthy C57/BL6 intact male mouse using both an ELISA technique and an ⁸⁹Zr-Labelled anti-PD-L1 mAb.

The study design used is shown in Table 1 below.

TABLE 1
Study Design matrix for anti-PD-L1 mAb mouse studies:

| | | | | Study 2 PK and |
|---|---|---|---|---|
| | | | | Biodistribution |
| | Study 1 | | Study 2 | using ⁸⁹Zr-Labelled |
| Routes of | Dose (mg/kg) per | Study 1 PK - | Dose (mg/kg) per | anti-PD-L1 mAb - |
| Administration | Administration | Single Dose | Administration | Single Dose |
| Group 1 | 10 | N = 30 (3 animals | 2 mg/kg | N = 18 (6 animals |
| SOFUSA ® | | per timepoint) | (40% ⁸⁹Zr-Labelled | per timepoint) |
| DoseConnect ™ | | | anti-PD-L1 mAb and | |
| Intra-Lymphatic | | | 60% anti-PD-L1 mAb) | |
| Delivery (Dorsal) | | | | |
| Group 2 | 10 | N = 30 (3 animals | 4 mg/kg | N = 18 (6 animals |
| Intravenous | | per timepoint) | (40% ⁸⁹Zr-Labelled | per timepoint) |
| Delivery (Tail | | | anti-PD-L1 mAb and | |
| Vein) | | | 60% anti-PD-L1 mAb) | |
| | | Response | | Response |
| | | Measurements | | Measurements |
| | | PK (anti-PD-L1 | | 1. PK (anti-PD-L1 |
| | | mAb drug levels | | mAb drug levels |
| | | in serum) | | in serum) |
| | | | | 2. Anti-PD-L1 organ |
| | | | | and Lymph Node |
| | | | | Concentrations |
| | | | | (axillary and |
| | | | | inguinal) |
| | | Measurement | | Measurement |
| | | Technique | | Technique |
| | | ELISA | | γ - Counts of |
| | | | | ⁸⁹Zr-Labelled |
| | | | | anti-PD-L1 mAb |

The Following Experimental Procedures were Used in Study 1: Pharmacokinetics of Anti-PD-L1 in C57/BL6 Healthy Mice.

Group 1: SOFUSA® DoseConnect™ Administration. Twenty-four hours prior to SOFUSA® DoseConnect™ administration, mice were anesthetized with isofluorane and the dorsal region was shaved and covered with depilatory cream (Nair Sensitive) for 8 minutes. The cream was then wiped off with warm, wet paper towels, followed by alcohol wipes. SOFUSA® DoseConnect™ was then applied to the dorsal region using a plastic shell with a skin adhesive. A hand-held applicator was then placed over the plastic shell to insert the microneedles into the skin. The operation of the device was as follows. The applicator strikes the microneedles with a post traveling at a velocity of 6 m/s. There is a total of 100 microneedles over the area of 66 mm². With the microneedles inserted in the skin, the syringe pump is started to deliver the drug. In these studies, the syringe pump was set at a constant rate of 150 μL/h and was run for on average 11 minutes to deliver the 10 mg/kg anti-PD-L1 mAb. The anti-PD-L1 mAb concentration was 10 mg/mL. There were 11 groups with 3 animals each for the blood collections. The timepoints were 15 minutes, 1, 8, 24 (1d), 48 (2d), 96 (4d), 168 (7d), 336 (14d), 504 (21d), or 672 (28d) hours post dose. All animals were euthanized on their timepoints and the blood collected using a cardiac draw. The blood samples were placed in EDTA tubes and spun at 2,500 rpm to collect the serum. The serum was evaluated for anti-PD-L1 mAb levels using an ELISA technique and the concentration at each timepoint was calculated as the average of the 3 animals.

Group 2: Intravenous Administration. In all animals, 25 μL of 10 mg/mL solution of anti-PD-L1 was injected in the tail vein. There were 11 groups with 3 animals each for the blood collections. The timepoints were 15 minutes, 1, 8, 24 (1d), 48 (2d), 96 (4d), 168 (7d), 336 (14d), 504 (21d), or 672 (28d) hours post dose. All animals were euthanized on their timepoints and the blood collected using a cardiac draw. The blood samples were placed in EDTA tubes and spun at 2,500 rpm to collect the serum. The serum was evaluated for anti-PD-L1 mAb levels using an ELISA technique and the concentration at each timepoint was calculated as the average of the 3 animals.

The Following Experimental Procedures were Used in Study 2—Pharmacokinetics and Biodistribution of 89Zr-Labelled Anti-PD-L1 mAb in C57/BL6 Healthy Mice.

Conjugation and 89Zr Labelling of anti-PD-L1 mAb. The anti-PD-L1 mAb was obtained from Sorrento Therapeutics, Inc. and conjugated with p-SCN-deferoxamine using method previously reported (Kam, K., Walsh, L. A., Bock, S. M., Fischer, K. E., Koval, M., Ross, R. F., and Desai, T. A., “Nanostructure-Mediated Transport of Biologics across Epithelial Tissue: Enhancing Permeability via Nanotopography”, Nano Lett. 2013, 13, 164-171; Vosjan M J, Perk L R, Visser G W, et al., “Conjugation and radiolabeling of monoclonal antibodies with zirconium-89 for PET imaging using the bifunctional chelate p-isothiocyanatobenzyl-desferrioxamine”, Nat Protoc. 2010; 5:739-743). Radiolabeling of Df-anti-PD-L1 mAb with ⁸⁹Zr was accomplished using traditional methods previously described and purified with PD-columns as described in Kam, K., Walsh, L. A., Bock, S. M., Fischer, K. E., Koval, M., Ross, R. F., and Desai, T. A., “Nanostructure-Mediated Transport of Biologics across Epithelial Tissue: Enhancing Permeability via Nanotopography”, Nano Lett. 2013, 13, 164-171.

Detailed ⁸⁹Zr Labeling Procedure. Coupling of the bifunctional chelate was performed as follows: (1) Pipette the required amount of mAb solution (maximum 1 ml; by preference between 2 and 10 mg/ml mAb) into an Eppendorf tube. Adjust the reaction mixture to a total volume of 1 ml by adding a sufficient amount of normal saline into the tube. Concentrations lower than 2 mg/ml will decrease the efficiency of the conjugation reaction, resulting in lower Df-mAb molar ratio. (2) Adjust pH of the mAb solution to pH 8.9-9.1 with 0.1 M Na2CO3 (maximum 0.1 ml). Alternatively, the desired pH for the reaction can be obtained by carrying out a buffer exchange of the mAb stock solution against 0.1 M sodium bicarbonate buffer (pH 9.0). (3) Dissolve Df-Bz-NCS in DMSO at a concentration between 2 and 5 mM (1.5-3.8 mg/ml) depending on the amount of mAb used. Add this to the protein solution to give a three-fold molar excess of the chelator over the molar amount of mAb and mix immediately. Keep the DMSO concentration below 2% in the reaction mixture. Typically, 20 μL (in steps of 5 μL) 2-10 mM Df-Bz-NCS (40-200 nmol) in DMSO is added to 2-10 mg intact antibody (13.2-66 nmol). In those cases, between 0.3-0.9 Df moieties will be coupled per antibody molecule. (4) Incubate the reaction for 30 min at 37° C. using a Thermomixer at 550 r.p.m. (5) In the meantime rinse a PD-10 column with 20 ml 5 mg/ml gentisic acid in 0.25 M sodium acetate (pH 5.4-5.6). (6) Pipette the conjugation reaction mixture onto the PD-10 column and discard the flow-through. (7) Pipette 1.5 ml of 5 mg/ml gentisic acid in 0.25 M sodium acetate (pH 5.4-5.6) onto the PD-10 column and discard the flow-through. (8) Pipette 2 ml of 5 mg/ml gentisic acid in 0.25 M sodium acetate (pH 5.4-5.6) onto the PD-10 column and collect the Df-protein. the Df-Bz-NCS-mAb can be stored at −20° C. until the day of radiolabeling for at least 2 weeks. Radiolabeling was performed as follows: (9) Pipette the required volume (=a) of [89Zr]Zr-oxalic acid solution (maximum 200 μL, typically 37-185 MBq) into a glass ‘reaction vial’. (10) While gently shaking, add 200 μL-a μ L (see Step 9) 1 M oxalic acid into the reaction vial. Subsequently, pipette 90 μL 2 M Na2CO3 into the reaction vial and incubate for 3 min at room temperature. (11) While gently shaking, pipette successively 0.30 ml of 0.5 M HEPES (pH 7.1-7.3), 0.71 ml of pre-modified mAb (typically 0.7-3.0 mg), and 0.70 ml 0.5 M HEPES (pH 7.1-7.3) into the reaction vial. The pH of the labeling reaction should be in the range of 6.8-7.2 for optimal labeling efficiency. (12) Incubate for 1 h at room temperature while gently shaking the reaction vial. Radiolabeling efficiency (typically >85%) can be determined by ITLC using chromatography strips and 20 mM citric acid (pH 4.9-5.1) (ITLC eluent) as solvent. A 0.5-2.0 μL aliquot of the reaction solution can be directly applied to the ITLC strip. Radiolabeled mAb (Rf=0.0-0.1). Any radioactivity Rf>0.1 represents radioactivity not bound to the mAb. Radiolabeling efficiency=CPM Rf 0.0-0.1 (CPM radiolabeled mAb)/CPM Rf 0.0-1.0 (CPM total)×100%. (13) Meanwhile, rinse a PD-10 column with 20 ml of 5 mg/ml gentisic acid in 0.25 M sodium acetate (pH 5.4-5.6). (14) After 1 h of incubation, pipette the reaction mixture onto the PD-10 column and discard the flow-through. (15) Pipette 1.5 ml of 5 mg/ml gentisic acid in 0.25 M sodium acetate (pH 5.4-5.6) onto the PD-10 column and discard the flow-through. (16) Pipette 2 ml of 5 mg/ml gentisic acid in 0.25 M sodium acetate (pH 5.4-5.6) onto the PD-10 column and collect the purified radiolabeled mAb. (17) Calculate the overall labeling yield: MBq 89Zr product vial (see Step 16)/MBq 89Zr starting activity (see Step 9)×100% and analyze the purified radiolabeled mAb by ITLC, HPLC and SDS-PAGE. When the radiochemical purity is greater than 95% it is ready for storage at 4° C. or dilution in 5 mg/ml gentisic acid in 0.25 M sodium acetate (pH5.4-5.6) for in vitro or in vivo studies. When the purity is <95% the PD-10 column purification should be repeated. The radiolabeled mAb is stable upon storage for 48 h (0.9%±0.4% dissociation of the initial bound 89Zr in a 37 MBq ml-1 89Zr-mAb solution at t=48 h, but presence of Cl-ions should be avoided). Gentisic acid is introduced during labeling and storage to minimize deterioration of the mAb integrity by radiation. The use of Cl-ions should be avoided, as radiation and subsequent radiolysis of water molecules form OCl-ions, which very specifically react with the SH-group of the enolized thiourea unit. Thus, the formed intermediary sulphenyl chloride bonds, and the sulphonyl chloride bonds arising upon further oxidation, are known to undertake a series of reactions, among which are coupling reactions and cleavage of methionyl peptide bonds. Therefore, the use of a 0.25 M sodium acetate buffer is strongly recommended.

Group 1: SOFUSA® DoseConnect™ Administration 89Zr-Labelled anti-PD-L1 mAb. Twenty-four hours prior to SOFUSA® DoseConnect™ administration, mice were anesthetized with isofluorane and the dorsal region was shaved and covered with depilatory cream (Nair Sensitive) for 8 minutes. The cream was then wiped off with warm, wet paper towels, followed by alcohol wipes. SOFUSA® DoseConnect™ was then applied to the dorsal region using a plastic shell with a skin adhesive. A hand-held applicator was then placed over the plastic shell to insert the microneedles into the skin. The operation of the device was as follows. The applicator strikes the microneedles with a post traveling at a velocity of 6 m/s. There is a total of 100 microneedles over the area of 66 mm². With the microneedles inserted in the skin, the syringe pump is started to deliver the drug. The syringe pump was set at a constant rate of 125 L/h and was run for an average 25 minutes to deliver the 2 mg/kg anti-PD-L1 mAb. The anti-PD-L1 mAb concentration was 1 mg/mL. The drug solution was 40% ⁸⁹Zr-Labelled anti-PD-L1 mAb and 60% anti-PD-L1 mAb. There were 3 groups with 6 animals each for the blood collections and necropsy. The timepoints were 1, 24 and 72 hours post dose and all animals were euthanized on their timepoints and the blood collected using a cardiac draw and necropsy performed. The blood samples, organs and lymph nodes were collected, and radioactivity measured using a 7-counter to calculate the concentration of anti-PD-L1 mAb versus the initial radioactive dose. The concentration at each timepoint was calculated as the average of the 6 animals.

Group 2: Intravenous Administration ⁸⁹Zr-Labelled anti-PD-L1 mAb. In all animals, 100 μL of 1 mg/mL solution of anti-PD-L1 was injected in the tail vein (4 mg/kg). The drug solution was 40% ⁸⁹Zr-Labelled anti-PD-L1 mAb and 60% anti-PD-L1 mAb. There were 3 groups with 6 animals each for the blood collections and necropsy. The timepoints were 1, 24 and 72 hours post dose and all animals were euthanized on their timepoints and the blood collected using a cardiac draw and necropsy performed. The blood samples, organs and lymph nodes were collected, and radioactivity measured using a γ-counter to calculate the concentration of anti-PD-L1 mAb versus the initial radioactive dose. The concentration at each timepoint was calculated as the average of the 6 animals.

Data analysis was performed as follows for Study 1—Pharmacokinetics of anti-PD-L1 in C57/BL6 Healthy Mice. In Study 1, there were 11 groups with 3 animals each for the blood collections. The timepoints were 15 minutes, 1, 8, 24 (1d), 48 (2d), 96 (4d), 168 (7d), 336 (14d), 504 (21d), or 672 (28d) hours post dose. The serum anti-PD-L1 mAb levels were determined using an ELISA technique. For each blood sample, triplicates were run and averaged for each animal. The concentration at each timepoint was then calculated using an average of the 3 animals.

Data analysis was performed as follows for Study 2—Pharmacokinetics and Biodistribution of ⁸⁹Zr-Labelled anti-PD-L1 mAb in C57/BL6 Healthy Mice. In Study 2, there were 3 timepoint groups with 6 animals each for both SOFUSA® DoseConnect™ lymphatic and intravenous delivery. The timepoints were 1, 24 and 72 hours post dose. At each timepoint all animals in the group were euthanized, and the radioactivity counted on the serum organs and lymph nodes. The concentration of anti-PD-L1 in the serum, organs, and lymph nodes was reported as a percentage of the initial dose (% ID) per unit mass of serum, organ tissue collected, or lymph node tissue collected. The values at each timepoint was than calculated as an average across all 6 animals. The total lymphatic delivery at each timepoint was and average across the axillary, inguinal, and brachial lymph nodes.

The Following Results were Observed for Study 1—Pharmacokinetics of Anti-PD-L1 in C57/BL6 Healthy Mice.

FIG. 7 is a graph reporting example PK curves for the SOFUSA® DoseConnect™ and intravenous administrations of anti-PD-L1 mAb in C57/BL6 mice. The dose given was 10 mg/kg and the average mouse weight was 25 g. The formulation concentration of anti-PD-L1 mAb was 10 mg/mL. The SOFUSA® DoseConnect® intra-lymphatic infusion was pumped at 150 μL/hr for an average of 11 minutes. The intravenous dose was injected through the tail vein. The serum concentrations at 24 and 48 hours from SOFUSA® DoseConnect™ delivery were not statistically different from the intravenous delivery.

The PK parameters for the curves in FIG. 7 are provided in Table 1. The bioavailability of the SOFUSA® DoseConnect™ intra-lymphatic delivery was calculated to be 20%. Bioavailability correlated to the infusion time and longer durations could be used to increase the value if necessary to achieve tumor inhibition.

TABLE 1
Comparison of anti-PD-L1 mAb PK parameters for SOFUSA ®
DoseConnect ™ and intravenous (IV) delivery:

| | | SOFUSA ® |
|---|---|---|
| PK Parameters | IV | DoseConnect ™ |
| Cₘₐₓ (ng/ml) | 85,000 | 31,000 |
| Tₘₐₓ | 5 mins | 24 hours |
| AUC₀₋₆₇₂ (ng-hr/ml) | 14,680,000 | 3,414,000 |
| BA (%) | N/A | 20 |

The Following Results were Observed for Study 2 Pharmacokinetics and Biodistribution of ⁸⁹Zr-Labelled Anti-PD-L1 mAb in C57/BL6 Healthy Mice.

FIG. 8 is a graph reporting example PK curves for the SOFUSA® DoseConnect™ and intravenous administrations of anti-PD-L1 mAb labelled with 89Zr radioisotope in C57/BL6 mice. The dose given was 2 mg/kg for SOFUSA® DoseConnect™ and 4 mg/kg for intravenous administration and the average mouse weight was 25 g. The formulation concentration of anti-PD-L1 mAb was 1 mg/mL and contained 40% ⁸⁹Zr-anti-PD-L1 and 60% anti-PD-L1 mAb. The SOFUSA® DoseConnect® intra-lymphatic infusion was pumped at 125 μL/hr for an average of 25 minutes. The intravenous dose of 100 μL was injected through the tail vein.

The serum concentrations were measured at 1, 24 and 72 hours. At 72 hours the PK curves from SOFUSA® DoseConnect™ and intravenous delivery are not statistically different. These results were slightly different from the PK curves obtained from the ELISA measurements. Specifically, the radiolabeling results shifted the intersection out almost another 24 hours. Other PK parameters were not calculated because the timepoints only went out to 72 hours.

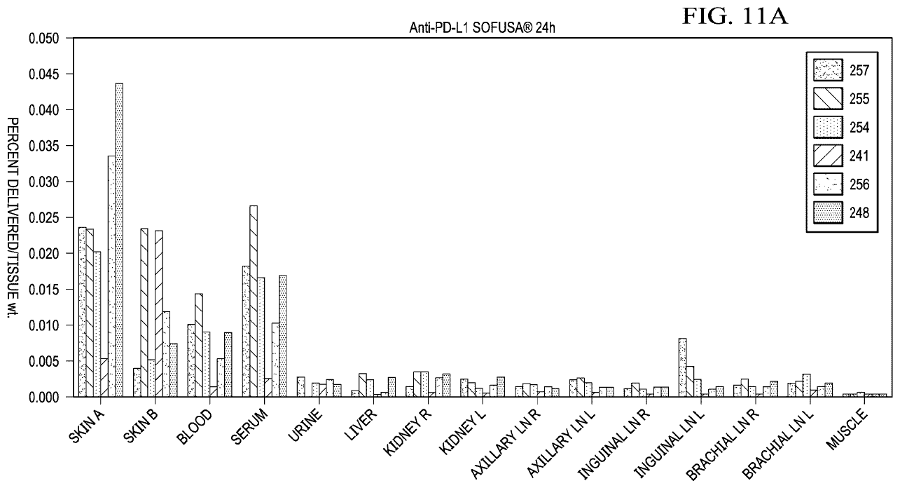

The biodistributions included the systemic organs (liver and kidneys) and lymph nodes (axial, inguinal, and brachial). The tissue was collected at 1, 24, and 72 hours. In FIG. 9, are the average lymph node levels across all 6 axial, iguinal, and brachial lymph nodes for SOFUSA® DoseConnect™ and intravenous delivery. The lymph node biodistributions showed that 184% more anti-PD-L1 was delivered on average across the axillary, inguinal, and brachial lymph nodes versus intravenous administration. The SOFUSA® DoseConnect™ treated lymph nodes also had the greatest levels at the 1-hour timepoint which turned out to be the lowest level for IV. The intravenous levels increased marginally over 72 hours while the SOFUSA® DoseConnect™ lymph node levels remained statistically constant.

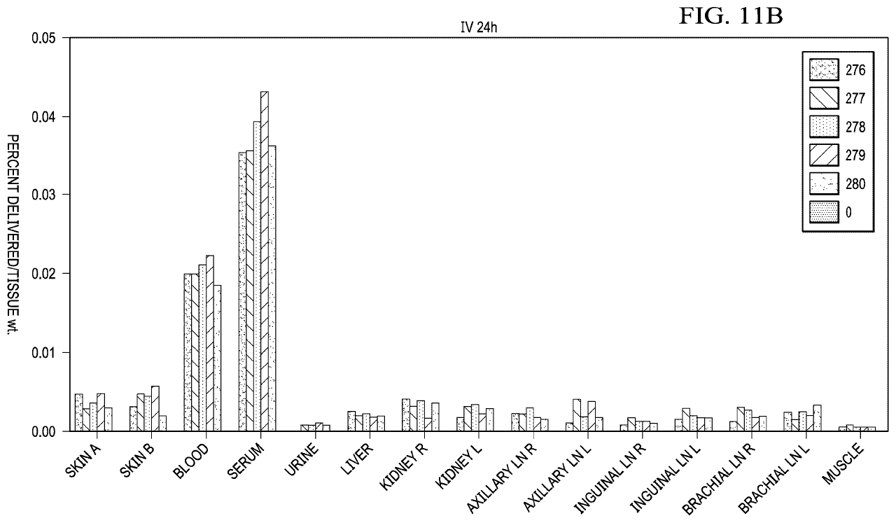

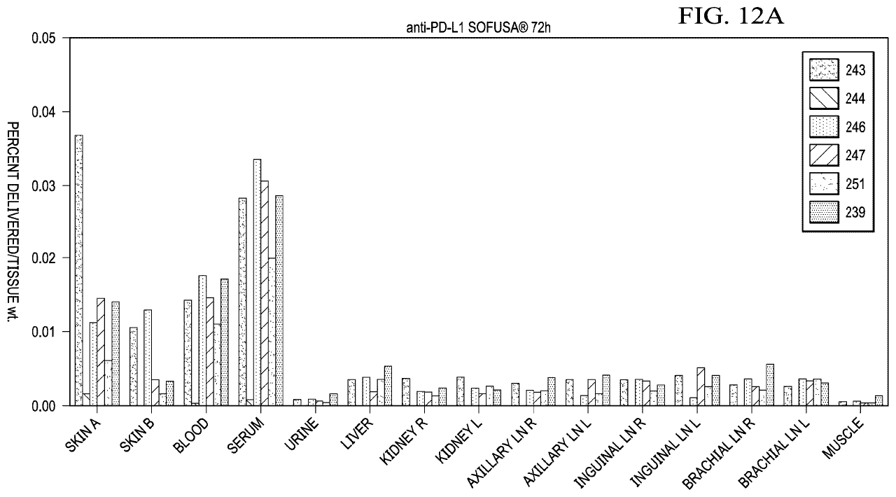

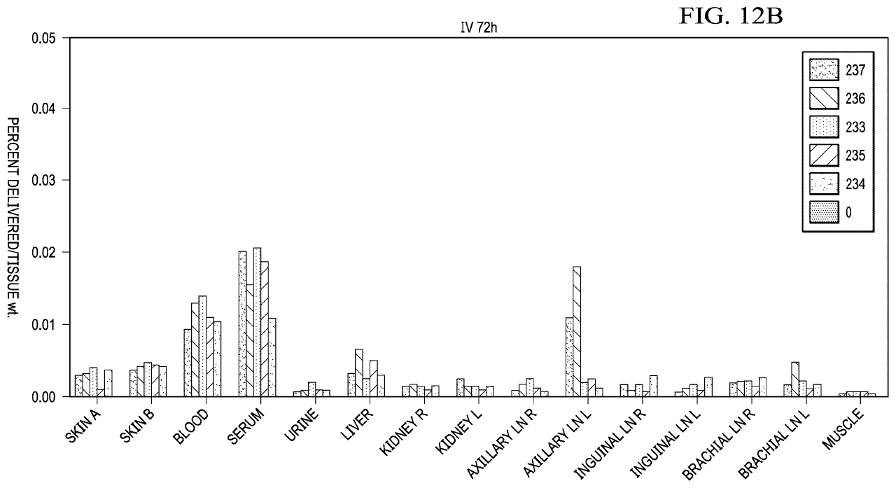

In FIG. 10A to FIG. 12B are the biodistribution results for ⁸⁹Zr-anti-PD-L1 mAb delivery in healthy C57/BL6 mice for SOFUSA® DoseConnect™ and intravenous administration. The systemic organs in the biodistribution study (liver and kidneys) had low concentrations at 1-hour from SOFUSA® DoseConnect™ delivery that increased over 72 hours to match intravenous levels. The intravenous organ concentrations were opposite with high values at 1-hour.

The results in this Example show that anti-PD-L1 mAb can be effectively delivered through regional intra-lymphatic delivery using the SOFUSA® DoseConnect™ microneedle device. The bioavailability was greater than 20% and could be increased with longer infusion times if required. The regional intra-lymphatic delivery showed real time delivery of anti-PD-L1 mAb to all the major lymph nodes at substantially higher concentration than intravenous administration with potentially lower systemic toxicity based on kidney and liver levels.

It is expected that optimized PK and intra-lymphatic delivery with SOFUSA® DoseConnect™ will translate into better tumor inhibition in mouse models and in human patients.

### Summary of Results Related to Pharmacokinetics:

With SOFUSA® the drug concentration rises to Cmax within approximately 24 hours and more than 99% is cleared in 28 days. With intravenous almost 83% is cleared in 28 days but the levels remain high and above 14 μg/mL.

No mice were lost during the study from SOFUSA® or intravenous delivery.

The mice skin tolerated the anti-PD-L1 mAb delivery from SOFUSA® without any significant erythema or edema.

For SOFUSA®, Tmax=24 Hours, Cmax=31,000 ng/mL, and BA=20%.

For intravenous Cmax=85,000 ng/mL.

### Summary of Results Related to Biodistribution:

SOFUSA® DoseConnect™ delivery of anti-PD-L1 mAb to the lymph system and lymph nodes was found to start immediately upon initiation of the SOFUSA® infusion.

SOFUSA® DoseConnect™ delivery demonstrated higher lymph node-to-blood concentrations for 30 hours post administration time of 1 hour.

Area Under the Curve measurements over 72 hours showed SOFUSA® DoseConnect™ delivered 184% more drug to the lymphatic system than intravenous administration.

Biodistribution results demonstrated sustained higher lymphatic concentrations with lower systemic exposure in other organ systems.

No mice were lost during the study from SOFUSA® DoseConnect™ or intravenous delivery.

The mice skin tolerated the anti-PD-L1 mAb delivery from SOFUSA® without any significant erythema or edema.

### Prophetic Example 4. Prophetic Pre-Clinical Study on Efficacy of SOFUSA® Delivery of Anti-PD-1 or Anti-PD-L1

The following methods may be used:

Orthotopic 4T1 animal model and immunotherapy treatment. 5×10⁵ luciferase-transfected 4T1 (4T1-luc) mouse mammary tumor cells (Li C W, Lim S O, Xia W, Lee H H, Chan L C, Kuo C W, et al. Glycosylation and stabilization of programmed death ligand-1 suppresses T-cell activity. Nat Commun. 2016; 7:12632) in 0.1 mL of PBS and Matrigel will be injected into the right caudal mammary fat pad of BALB/C mice. At day 11, animals will be separated into one of five treatment groups that received (1) 10 mg/kg anti-PD-1 mAb in 0.05 mL PBS intraperitoneal injection (i.p.); (2) 10 mg/kg anti-PD-1 mAb in 0.05 mL PBS infused via SOFUSA® DoseConnect™; and (3) 10 mg/kg anti-PD-L1 mAb in 0.05 mL PBS intraperitoneal injection (i.p.); (2) 10 mg/kg anti-PD-L1 mAb in 0.05 mL PBS infused via SOFUSA® DoseConnect™; and (5) 10 mg/kg isotype control antibody on days 11, 15, 19, and 23 p.i. All the cohorts will have similar tumor volumes at start of dosing on day 11. Animals with tumor volumes that are statistically different from the group at day 11 will not be included in the analysis. Study endpoint will be 30 days post-implant or the tumor exceeds 20 mm in any dimension, whichever comes first.

SOFUSA® DoseConnect™ lymphatic infusion device. In animals, 50 μL of 4.5 mg/mL solution of anti-PD-1 mAb or anti-PD-L1 mAb will be infused over an hour on the right dorso-lateral side of isoflurane anesthetized animals. Lymphatic imaging will be performed non-invasive near-infrared fluorescence imaging as previously described (Sevick-Muraca E M, Kwon S, Rasmussen J C. Emerging lymphatic imaging technologies for mouse and man. J Clin Invest. 2014; 124:905-14).

Assessment of tumor burden. At days 4, 8, 11, 15, 19, 23, 26, and 30 days post-implant (p.i.), short (D1) and long (D2) tumor dimensions will be assessed from caliper measured and volumes (V) computed from 0.5×D1²×D2 (Faustino-Rocha A, Oliveira P A, Pinho-Oliveira J, Teixeira-Guedes C, Soares-Maia R, da Costa R G, et al. Estimation of rat mammary tumor volume using caliper and ultrasonography measurements. Lab Anim (NY). 2013; 42:217-24). At days 16, 23, and 30 days p.i. tumor burden will be assessed in a subset of animals using bioluminescence with a custom build, bioluminescence device. In vivo bioluminescence images will be acquired 10 min after i.p. administration of D-luciferin (150 mg/kg in 200 μL of PBS; Goldbio). For ex vivo bioluminescence imaging at 30 days p.i., organs will be removed immediately after the second D-luciferin administration (approximately 20 min after the first D-luciferin injection), incubated in D-luciferin solution, and imaged. Tissues will be subsequently evaluated through gross examination and histology.

Immunohistochemical staining. Tissue samples will be embedded in paraffin and 4 m sections used in all staining procedures. Following paraffin removal and antigen retrieval using citrate buffer, tissues will be incubated with H₂O₂, blocked with 5% normal goat serum albumin, and stained with rat anti-mouse CD8 antibody (eBioscience™) and biotin-anti rat secondary antibody (Vector Labs). The Vectastain Elite ABC system for peroxidase and DAB as chromogens will be used before tissues were counter-stained with hematoxylin (Vector Labs). CD8 expression will be examined at ×63 magnification (Zeiss Axio).

Data analysis and statistics. Tumor growth data may be presented as average volumes±standard error (SE). Statistical analysis may be performed with Microsoft Excel and volume data from individual time points may be analyzed by unpaired 1-tailed Student's t-test with the significance level set at p<0.05. Upon euthanasia, tissues will be collected and examined for lung, liver, and LN metastases, and each animal will be assessed for the number of lung lesions. Differences between the numbers of animals with and without metastases will be statistically evaluated by z-test with the level of significance set at p<0.05.

The following results are expected:

### Lymphatic Delivery of Anti-PD-1 or Anti-PD-L1 is Expected to Improve Anti-Tumor Response in an Orthotopic Mammary Carcinoma Murine Model.

In BALB/C mice with orthotopic implants of 4T1-luc mouse mammary in the right caudal mammary fat pad, SOFUSA® DoseConnect™ will be used to infuse 10 mg/kg anti-PD-1 or anti-PD-L1 in 0.05 mL PBS on the right lateral side with infusion rates of 100 μL/h on days 11, 15, 19, and 23 post implant (p.i). Tumor growth rates and, in a subset of animals, bioluminescence imaging of tumor burden will be compared to additional groups of tumor bearing animals receiving either 10 mg/kg anti-PD-1 or anti-PD-L1 or isotype control antibody through intraperitoneal (i.p.) injection on days 11, 15, 19, and 23 p.i.

Bioluminescence imaging in a subset of animals is expected to show anti-PD-1 or anti-PD-L1 will arrest, or slow, or stop, tumor growth and LN, bone, and lung metastases, such as at day 23 and 30 p.i., and from ex-vivo imaging at the 30 day study endpoint, the amount of distant metastases. Of the animals dosed with SOFUSA® DoseConnect™ infusion, it is expected that a proportion of animals, such as at least, or at least about, 30%, 40%, 50%, 60%, 70%, 80%, 90%, 95%, or 99%. will show a complete response, such as determined by undetectable primary tumor volume by caliper measurement, or by any other method identifiable in the art. No animals receiving i.p. administration of anti-PD-1 or i.p. administration of anti-PD-L1 or isotype control are expected to show a complete response, consistent with lymphatic delivery of anti-PD-1 or anti-PD-L1 to TDLNs potentiating early and robust anti-tumor activity. In addition, statistically significantly fewer animals, such as up to 20%, 25%, 30%, 35%, 40%, 45%, 50%, 55%, 60%, 65%, 70%, or 75% fewer animals are expected to show distant metastases, such as lung, bone, or LN metastases, in SOFUSA® DoseConnect™ infused animals compared to systemically dosed and isotype control groups. These expected results suggest that despite the regional delivery of drug, there is an effective abscopal anti-tumor response from regional SOFUSA® DoseConnect™ dosing in comparison to systemic dosing. This expected result is consistent with (i) the greater drug exposure to naïve T cells in TDLNs with lymphatic delivery and (ii) a more effective activation of T-cells against tumor Ags which once educated, leave TDLNs to mount a robust systemic anti-tumor response.

Tumor bearing animals receiving SOFUSA® DoseConnect™ infusion of anti-PD-1 or anti-PD-L1 are expected to exhibit significantly reduced tumor growth that occurs from an earlier time point when compared to that observed in animals receiving i.p. injection of anti-PD-1 or anti-PD-L1. For example, SOFUSA® DoseConnect™ infusion of anti-PD-1 or anti-PD-L1 is expected to exhibit significantly reduced tumor growth over a period of time, such as at day 15 p.i. and onwards, when compared to animals dosed with isotype control antibody. In comparison, tumor bearing animals receiving i.p. injection of anti-PD-1 or anti-PD-L1 are expected to exhibit statistically significant reduced tumor growth from a later time point, such as on day 19 p.i. and onwards when compared to animals administered with isotype control antibody. Beginning from a time point, such as at day 15 p.i., the tumor volumes of animals having received a first round of anti-PD-1 or anti-PD-L1 via SOFUSA® DoseConnect™ infusion are expected to be significantly smaller than tumor volumes in animals that received anti-PD-1 or anti-PD-L1 systemically. This expected result is consistent with an earlier anti-tumor response in animals dosed regionally with SOFUSA® DoseConnect™ infusion into the lymphatics as compared to those receiving drug systemically.

IHC staining of the subset of SOFUSA® DoseConnect™ anti-PD-1-dosed or anti-PD-L1-dosed animals that have a residual primary tumor at study endpoint, are expected to show statistically greater number, e.g. an increase of up to 1.5-fold, 2-fold, 2.5-fold, or more, of tumor-infiltrating lymphocytes (TILs) in the primary tumors compared to controls or to animals dosed systemically. For example, it may be expected that an increase of 50% TILs in the primary tumors is observed following intravenous administration of an anti-PD-1 or anti-PD-L1 therapeutic agent, compared to an increase of 100% TILs in the primary tumors following lymphatic administration.

The impact of lymphatic delivery on tumor responses expected in this preclinical study may be attenuated in the small, quadrupedal preclinical tumor models as compared to bipedal non-human primates or patients. Systemic administration of monoclonal antibodies in rodent studies are commonly performed with i.p. injection for effective uptake by the plentiful lymphatics in the peritoneal cavity that promptly empties into the venous system. As a result, i.p. administration largely approximates the same pharmacokinetic and pharmacodynamic profiles seen with i.v. injection. While i.p. administration may escape the exposure to tumor draining LNs seen in this lymphatic delivery study, the i.p. route of administration nonetheless uses the truncal lymphatics to deliver drug to the blood circulation. As a result of the exposure in the lymphatic compartment, the anti-tumor responses from i.p. administration in rodents may be expected to overpredict those from i.v. administration in humans. In addition to the attenuated response in rodent models, adverse immune responses to immunotherapies are generally non-existent in rodents, further requiring other pre-clinical and clinical investigation, described in the present disclosure, to understand whether lymphatic delivery can ameliorate irAEs.

### Example 5. A Multi-Phase Exploratory Toxicity and Toxicokinetics Study of Anti-PD1 Monoclonal Antibody STI-A1110 Following Lymphatic Administration in Cynomolgus Monkeys (Non-GLP)

The objectives of this two-phase study were to 1) evaluate the responses in cynomolgus monkeys to lymphatic drug delivery of anti-PD1 Monoclonal Antibody STI-A1110 at various locations and exposure levels at 1 hour postdose (Phase I) and 2) determine the potential toxicity and compare toxicokinetic profiles in naïve cynomolgus monkeys at various time points following intravenous (IV) and lymphatic drug delivery (Phase II). SOFUSA® DoseConnect™ was used for lymphatic delivery.

It is expected that the results described in this Example in relation to an anti-PD1 antibody may be equally applicable to an anti-PD-L1 antibody.

The following experimental procedures were used.

Testing Facility: Testing was performed at Altasciences Preclinical Seattle LLC, Everett, WA.

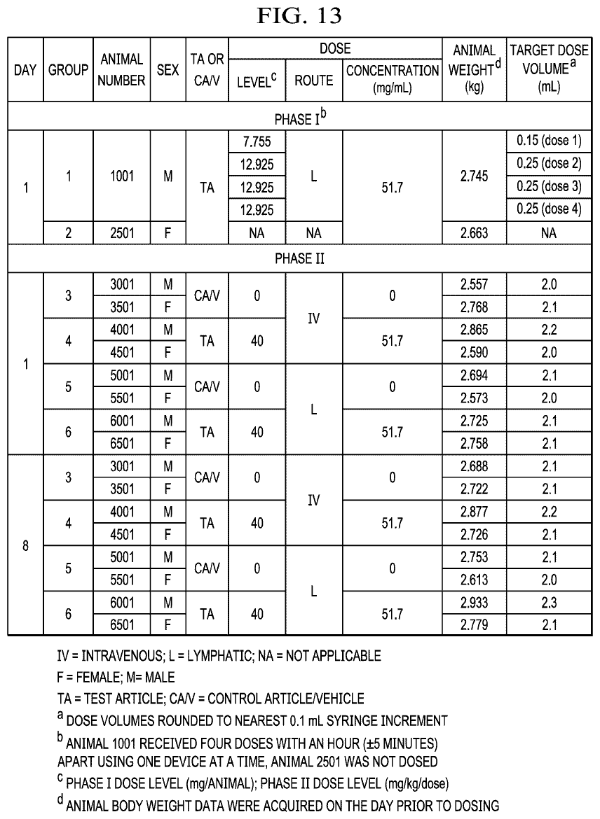

Animals. One male and one female cynomolgus monkeys (Macaca fascicularis), and five male and five female cynomolgus monkeys (Macaca fascicularis), which were naïve and confirmed to have no known exposure to monoclonal antibodies and acceptable results in veterinary physical examinations, were assigned to Phase I and Phase II, respectively. Animals were selected from the stock colony at Testing Facility and were randomly assigned to 6 treatment groups, as shown in FIG. 13. The remaining male and female served as the spares.

Animals were acclimated to the study room for 14 days prior to lymphatic drug delivery on Day 1 in Phase I. Animals were also acclimated to the study room for 14 days prior to intravenous (IV) infusion as well as lymphatic dose administration on Day 1 and Day 8 in Phase II. The in-life phase of the study was completed on Day 2 in Phase I, and Day 22 in Phase II. The spare animals were returned to the stock colony after dosing on Day 1, followed by the remaining study animals after the completion of the in-life portion of the study.

Dose preparation. The test article was anti-PD-1 monoclonal antibody STI-A1110 (Sorrento Pharmaceuticals). The control article/vehicle was 20 mM sodium phosphate, 100 mM sodium chloride, 200 mM sucrose, 0.05% PS80, pH 7.2 (±0.2]).

For Phase I, on Day 1, 2 mL of test article was removed from 2 to 8° C. and maintained at ambient temperature for at least 30 minutes prior to use.

For Phase II, on Day 1, 16 mL of test article and 19.8 mL of control article/vehicle were removed from 2 to 8° C. and maintained at ambient temperature for at least 30 minutes prior to use. On Day 8, 20 mL of test article and 21.5 mL of control article/vehicle were removed from 2 to 8° C. and maintained at ambient temperature for at least 30 minutes prior to use.

Target dose volumes were calculated based on the body weights measured on the day prior to dosing.

Dose administration. For Phase I: On Day 1, test article was administered to Animal 1001 (Group 1 male) at four different lymphatic locations, as indicated in FIG. 13. The animal was anesthetized for dose administration. The four doses were administered to the upper right forelimb (dose 1, 0.15 mL/hr), right thigh (lateral, dose 2, 0.25 mL/hr), left medial thigh (dose 3, 0.25 mL/hr) and left medial thigh (dose 4, closer to the knee than dose 3, 0.25 mL/hr), respectively, with an hour (±5 minutes) apart using one device at a time. The selected sites for application of the drug delivery device were clipped prior to dosing. Animal 2501 (Group 2 female), was not dosed.

For Phase II: Animals in Groups 3 and 4 were dosed into a peripheral vein via an 8-minute (±30 seconds) intravenous (IV) infusion using a pump at dose levels of 0 or 40 mg/kg once daily on Days 1 and 8 as indicated in FIG. 13. Infusion lines were flushed with 1 mL of control article/vehicle (20 mM sodium phosphate, 100 mM sodium chloride, 200 mM sucrose, 0.05% PS80, pH 7.2 [±0.2]) to ensure complete delivery of the intended dose volume. The dosing volume was constant among groups, at 0.77 mL/kg.

Control or test articles were administered to anesthetized animals in Groups 5 or 6 using 4 lymphatic drug delivery devices simultaneously on Days 1 and 8, as indicated in FIG. 13. Dose duration for animals in Groups 5 and 6 was over 2.5 hours. On Day 1, the left shoulder, upper left back and two upper left hindlimbs were used as the four dosing sites in Animals 5001 (Group 5 male) and 6001 (Group 6 male); the left hip, left lower back directly above the left hip site, the left shoulder along the back and the upper left back were used in Animal 5501 (Group 5 female); and the left hip, upper left hindlimb, left upper forelimb and upper left back were dosed in Animal 6501 (Group 6 female). On Day 8, doses were administered on different limbs and/or dorsal back using four devices simultaneously.

Sample Collection, Processing, Storage, and Transfer. Animals were fasted for at least 4 hours prior to each series of collections that included specimens for serum chemistry. In these instances, associated clinical pathology evaluations were from fasted animals. All blood specimens were collected via a single draw from a peripheral vein of anesthetized or restrained, conscious animals using a butterfly infusion set and disposable syringe.

Approximately 0.5 mL was placed in K2EDTA BD MAP tubes for hematology, 1 mL was placed into serum separator tubes (SSTs) for serum chemistry, 0.9 mL was placed into 3.2% sodium citrate for coagulation analyses, and 1 mL was placed in serum separator tubes for serum toxicokinetic (TK) analysis.

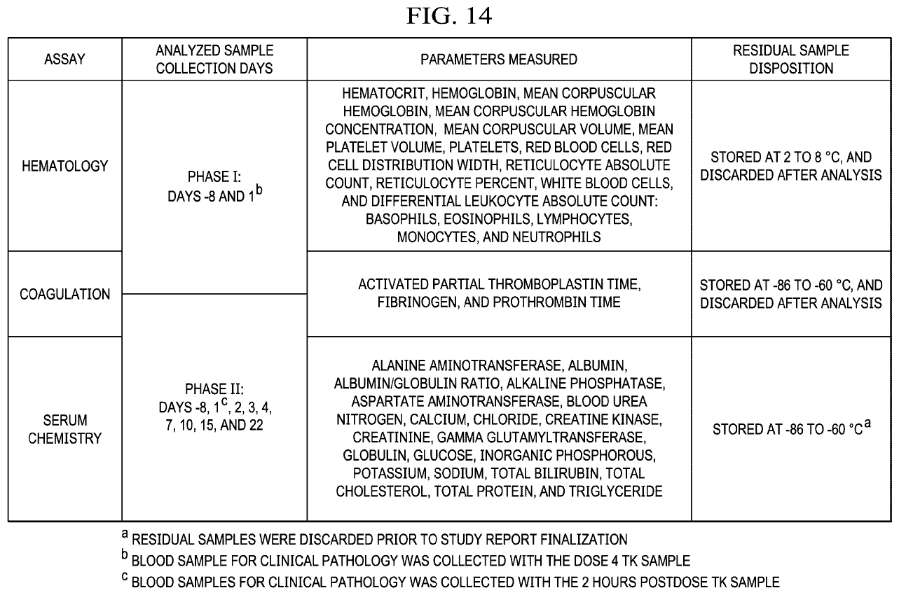

Clinical Pathology. Hematology, coagulation, and serum chemistry were analyzed. For each assay, the analyzed sample collection days, parameters measured, and sample disposition are reported in FIG. 14.

Toxicokinetic (TK) samples. For Phase I: Blood samples were collected once on Day −8, and just prior to device removal at 1 hour post start of each dose administration.

For Phase II: Blood samples were collected once on Day −8, and at 0.5, 1, 2, 4, 8, 24, 48, 72, 144, 216, 336 and 504 hours post the end of intravenous infusion on Day 1 in Groups 3 and 4. Blood samples were collected once on Day −8, and at 2, 4, 8, 24, 48, 72, 144, 216, 336 and 504 hours post the start of Day 1 dose for Animals 5001 and 6001. Blood samples were collected once on Day −8, and at 0.5, 1, 2, 4, 8, 24, 48, 72, 144, 216, 336 and 504 hours post the start of Day 1 dose for Animals 5501 and 6501.

Approximately 1 mL of blood for TK analysis was deposited into serum separator tubes (SSTs) and allowed to stabilize at room temperature for at least 30 minutes before centrifugation at 2000×g in a refrigerated centrifuge (2 to 8° C.) for 15 minutes to obtain serum. Approximately 250 μL of serum were transferred to a polypropylene tube (aliquot 1), and the remaining serum was transferred to a different tube (aliquot 2), and placed immediately on dry ice prior to storage at −86 to −60° C.

Serum (aliquot 1) samples for TK analyses collected in Phase I and Phase II were shipped to Sorrento Therapeutics Inc. The second set of serum samples were stored at −86 to −60° C. and were shipped to Sorrento Therapeutics Inc.

The following results were observed.

Clinical Observations and Veterinary Interventions. There were no test article-related observations in this study.

Body Weights. There was no test article-related change in body weight.

Hematology, Coagulation, and Serum Chemistry. Hematology, coagulation and serum chemistry samples were taken from all animals and were processed according to the Testing Facility SOPs.

For Phase I, no changes in clinical pathology were attributed to the test article administration.

For Phase II intravenous infusion dosing, test article-related changes in clinical pathology consisted of moderately to markedly decreased platelet counts, mildly increased fibrinogen, and minimally increased globulin in animals dosed at 40 mg/kg. Decreased platelet counts occurred following the Day 1 and Day 8 dosing with nadir occurring two to three days postdose (Days 3, 4, and/or 10). Concurrent increased mean platelet volume and large platelets (female only) were consistent with release of less mature platelets (early regenerative response) and were suggestive of increased platelet loss. The magnitude of the decreased platelet counts was considered adverse, but was transient, did not result in increased tendency towards bleeding (lack of petechiae, purpura or hemorrhage from any orifice) and had evidence of reversibility by Day 22. Increased fibrinogen and globulin were consistent with an inflammatory response and had evidence of partial or complete reversibility by Day 22.

For Phase II lymphatic dosing, test article-related changes in clinical pathology consisted of mildly increased fibrinogen in the male dosed at 40 mg/kg, were consistent with an inflammatory response, and had evidence of reversibility by Day 22. No hematology or clinical chemistry changes were attributed to the test article in animals administered 40 mg/kg/dose via lymphatic delivery.

Changes attributed to test article anti-PD-1 mAb STI-A1110 administration included transient, moderate to marked platelet count decrease in the intravenous (IV) infusion males and females (Group 4) that was not associated with increased bleeding (petechiae, purpura or overt hemorrhage from any orifice). In addition, transient, mildly increased fibrinogen and minimally increased globulin in animals dosed at 40 mg/kg via intravenous route, as well as mildly increased fibrinogen in the lymphatic administration (Group 6) male were consistent with an inflammatory response.

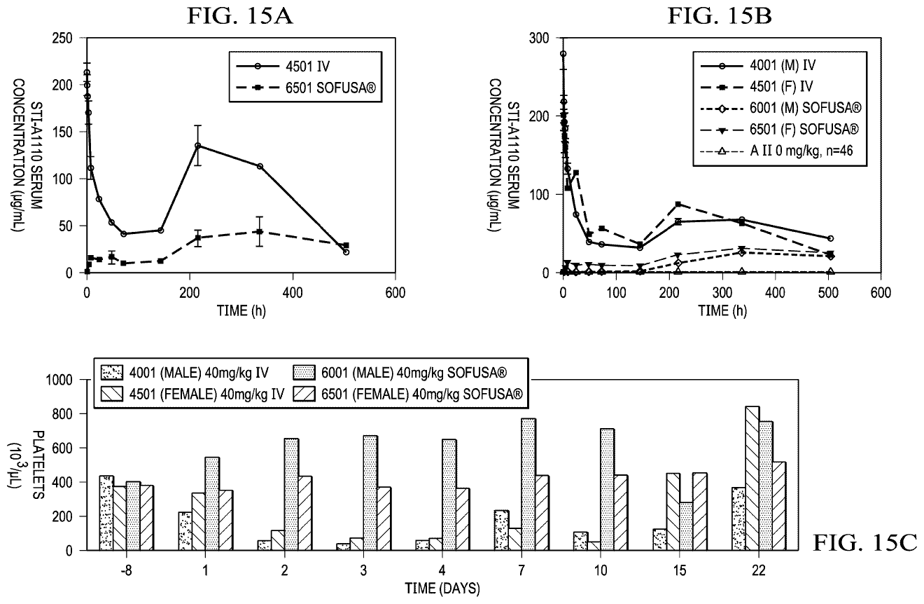

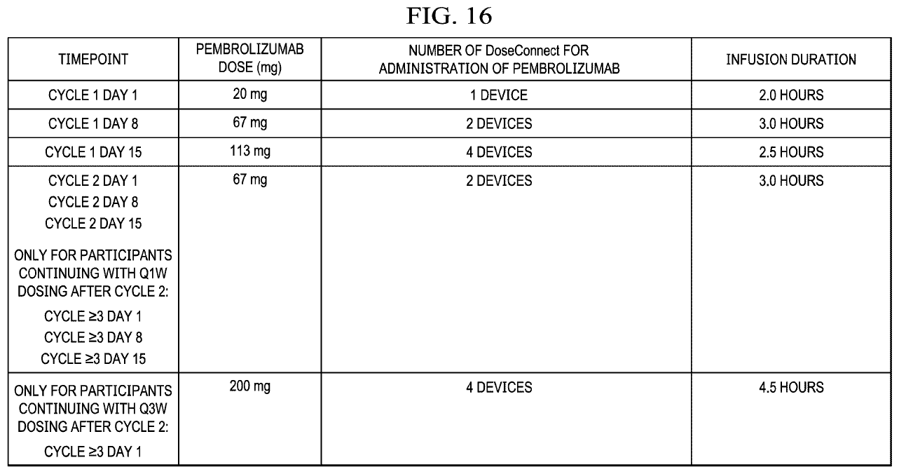

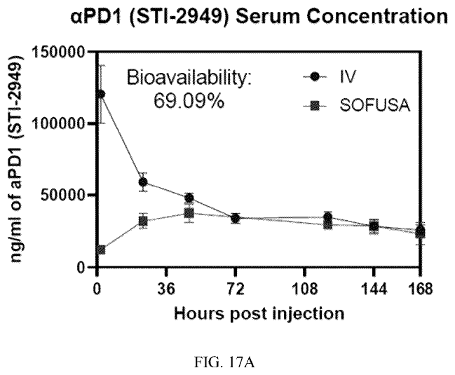

Consistent dosing/PK profiles were observed with SOFUSA® DoseConnect™ dosing without the platelet reduction seen with intravenous administration (FIG. 15C).

Pharmacokinetic (PK) enzyme-linked immunosorbent assay (ELISA) results of serum concentrations of anti-PD1 monoclonal antibody STI-A1110. FIG. 15A shows pharmacokinetic (PK) enzyme-linked immunosorbent assay (ELISA) results of serum concentrations of anti-PD1 monoclonal antibody STI-A1110 (Sorrento Pharmaceuticals) following intravenous dosing in monkey 4501 and lymphatic delivery using SOFUSA® DoseConnect™ in monkey 6501.

As shown in FIG. 15A and FIG. 15B, intravenous delivery of anti-PD1 monoclonal antibody STI-A1110 resulted in an immediate increase in concentration of serum anti-PD1 monoclonal antibody STI-A1110, followed by a rapid decrease, particularly following administration on Day 1. In contrast, lymphatic delivery of anti-PD1 monoclonal antibody STI-A1110 using SOFUSA® DoseConnect™ resulted in more consistent serum levels throughout the serum sampling period, with less variation in serum concentration of anti-PD1 monoclonal antibody STI-A1110.

AUC₀₋₅₀₀ hours was 41,300 ug/hr/ml for intravenous administration compared to 14,550 ug/hr/ml for lymphatic delivery. Bioavailability of anti-PD1 monoclonal antibody STI-A1110 administered lymphatically was 35%. Following administration on Day 1, Tmax was increased from 5 minutes following intravenous administration on Day 1, to 48 hours following lymphatic administration on Day 1.

### Prophetic Example 6. A Phase 1B, Pilot Study to Assess the Pharmacodynamics, Pharmacokinetics, Safety, and Activity of KEYTRUDA® (Pembrolizumab) (Merck Sharp & Dohme Corp., New Jersey) Administered Intra-Lymphatically Using SOFUSA® DoseConnect™ in Patients with Relapsed or Refractory Cutaneous T-Cell Lymphoma (CTCL)

This prophetic example describes an example of a planned phase 1B, pilot study to assess the pharmacodynamics, pharmacokinetics, safety, and activity of an anti-PD-1 antibody, e.g. pembrolizumab administered intra-lymphatically using the SOFUSA® DoseConnect™ device in patients with relapsed or refractory cutaneous T-cell lymphoma (CTCL).

It is expected that the results described in this Example in relation to an anti-PD-L1 antibody may be equally applicable to an anti-PD-1 antibody.

Cutaneous T-cell lymphomas (CTCLs) is a group of non-Hodgkin's Lymphomas of mature T-cells that are primarily present in the skin, and at times progress to involve the lymph nodes, blood and visceral organs (Swerdlow S H, Campo E, Pileri S A, et al. The 2016 revision of the World Health Organization classification of lymphoid neoplasms. Blood 2016; 127:2375-2390). The two most common subtypes of CTCL are mycosis fungoides (MF) and Sezary syndrome (SS), which constitute the majority of diagnoses. Mycosis fungoides is the most common subtype of CTCL with primary cutaneous involvement, accounting for 50%-70% of CTCLs (Paulli M, Berti E, Rosso R, et al. CD30/Ki-1-positive lymphoproliferative disorders of the skin-clinicopathologic correlation and statistical analysis of 86 cases: a multicentric study from the European Organization for Research and Treatment of Cancer Cutaneous Lymphoma Project Group. J Clin Oncol 1995; 13:1343-1354; Vergier B, Beylot-Barry M, Pulford K, et al. Statistical evaluation of diagnostic and prognostic features of CD30+ cutaneous lymphoproliferative disorders: a clinicopathologic study of 65 cases. Am J Surg Pathol 1998; 22:1192-1202.). Sezary syndrome is a rare erythrodermic, leukemic variant of CTCL characterized by significant blood involvement and lymphadenopathy and only accounts for about 1% to 3% of CTCLs (Id.). As a whole, CTCL is quite rare, constituting ˜4% of NHL diagnoses in the United States (Korgavkar, K., M. Xiong and M. Weinstock (2013). “Changing incidence trends of cutaneous T-cell lymphoma.” JAMA Dermatol 149(11): 1295-1299.).

Study rationale. Initial treatment in patients with patch/plaque CTCL disease consists of skin-directed therapies (localized or generalized including PUVA) with the addition of milder systemic therapies for refractory, persistent or progressive disease. Patients who do not respond to biologic therapy or those with very aggressive or extracutaneous disease may be treated with combination chemotherapy. With the potential exception of allogeneic stem-cell transplantation, there are no curative therapies for CTCL. Long-term curative treatment options have long been a challenge in CTCL. For relapsed or refractory disease, participation in clinical trials is recommended (National Comprehensive Cancer Network. Primary Cutaneous Lymphomas (Version 2.2019). https://www.nccn.org/professionals/physician_gls/pdf/primary_cutaneous.pdf.

In a study of biopsies taken from CTCL patients, CD4+ CTCL populations contained more T cells expressing PD-1, CTLA-4, and LAG-3 compared to normal skin. CTCL populations also contained more T cells expressing the inducible T-cell costimulator (ICOS), a marker of T-cell activation. Advanced T3/T4-stage samples expressed higher levels of mRNA from checkpoint inhibition genes compared with T1/T2 stage patients or healthy controls. Exhaustion of activated T cells is therefore a hallmark of both CD4+ and CD8+ T cells isolated from the lesioned skin of patients with CTCL (Querfeld C, Zain J M, Wakefield D L, et al. Phase 1/2 Trial of Durvalumab and Lenalidomide in Patients with Cutaneous T Cell Lymphoma (CTCL): Preliminary Results of Phase I Results and Correlative Studies, Blood 2018a 132:2931; Querfeld C (2018b, December). Phase 1/2 Trial of Durvalumab and Lenalidomide in Patients with Cutaneous T Cell Lymphoma (CTCL): Preliminary Results of Phase I Results and Correlative Studies. Oral presentation at the annual meeting of the American Society of Hematology. San Diego, CA). A phase 2, single-arm study led by the Cancer Immunotherapy Trials Network (CITN) evaluating pembrolizumab, an antibody to PD-1, showed promising results in people with higher stages of relapsed or refractory MF and SS (Khodadoust M, Rook A H, Porcu P, et al. Pembrolizumab for treatment of relapsed/refractory mycosis fungoides and Sezary syndrome: clinical efficacy in a CITN multicenter phase 2 study. Blood. 2016; 128(22):181). In this study 24 patients with MF/SS stages IB-IV treated with at least 1 prior systemic therapy were treated with intravenous pembrolizumab 2 mg/kg every 3 weeks for up to 2 years. The ORR was 38% (n=24) with a median time to treatment response of 11 weeks. One patient had a complete response, 8 patients had a partial response, 9 had stable disease, and 6 patients had PD. At 32 weeks, 8 of the 9 responses were ongoing. Based upon these results, although not approved, pembrolizumab is now listed in national guidelines as a treatment option for CTCL treatment. In a separate small clinical trial of durvalumab (anti-PD-L1) plus lenalidomide in patients with CTCL, it has been reported that this combination is also active in improving skin disease and producing partial responses. Strong PD-L1 and ICOS expression is observed from non-responders. Detectable levels of PD-L1, but low levels of ICOS is observed in responding patients. Quantitative super-resolution microscopy detected nanoscale clusters of PD-1 in T cells from responders and no PD-1 clustering was observed in T cells from non-responders (Querfeld C, Zain J M, Wakefield D L, et al. Phase 1/2 Trial of Durvalumab and Lenalidomide in Patients with Cutaneous T Cell Lymphoma (CTCL): Preliminary Results of Phase I Results and Correlative Studies, Blood 2018a 132:2931). The combination appeared to suppress the expression of PD-L1 and ICOS, possibly through down regulation of STAT1 and STAT3 (Querfeld C (2018b, December). Phase 1/2 Trial of Durvalumab and Lenalidomide in Patients with Cutaneous T Cell Lymphoma (CTCL): Preliminary Results of Phase I Results and Correlative Studies. Oral presentation at the annual meeting of the American Society of Hematology. San Diego, CA).

Regional delivery using a nanotopography-based microneedle array is expected to improve checkpoint blockade immunotherapy by reducing systemic drug exposure and maximizing drug delivery to the tumor bed in the skin and to tumor draining LNs where tumor antigens are present. In this pilot study, pembrolizumab will be administered via DoseConnect in patient with CTCL to assess through pharmacodynamic assessment in the tumor tissue if lymphatic delivery of pembrolizumab using SOFUSA® is feasible. The choice of CTCL is based on accessibility of tumor cells for pharmacodynamic measurements.

In clinical studies of single agent pembrolizumab, the most common AEs (reported in ≥20% of patients) were fatigue, musculoskeletal pain, decreased appetite, pruritus, diarrhea, nausea, rash, pyrexia, cough, dyspnea, constipation, pain, and abdominal pain (KEYTRUDA® (pembrolizumab) [package insert]. Merck & Co., Inc., Whitehouse Station, NJ; 2019). These common AEs were generally Grade 1 to 2.

Pembrolizumab can cause immune-related AEs. A wide range of immune related AEs, often in the form of autoimmune disease, is expected to occur at a low incidence (Id.). In the CITN experience of pembrolizumab in CTCL, AEs were consistent with those seen in prior studies of pembrolizumab with the exception of an immune-related skin flare reaction seen in 6 patients (2 patients with grade 2 and 4 patients with grade 3) which occurred exclusively in patients with SS (6/15; 40%) (Khodadoust M, Rook A H, Porcu P, et al. Pembrolizumab for treatment of relapsed/refractory mycosis fungoides and Sezary syndrome: clinical efficacy in a CITN multicenter phase 2 study. Blood. 2016; 128(22):181). Participants enrolled in this study may be monitored for signs and symptoms of immune-related AEs and treated with steroid and other supportive measures as required in accordance with published guidelines (Brahmer J R, Lacchetti C, Schneider B J et al. Management of Immune-Related Adverse Events in Patients Treated with Immune Checkpoint Inhibitor Therapy: American Society of Clinical Oncology Clinical Practice Guideline. Journal of Clinical Oncology 2018 36:17, 1714-1768.). Pembrolizumab can cause severe or life-threatening infusion-related reactions, including hypersensitivity and anaphylaxis, which have been reported in 6 (0.2%) of 2799 patients receiving pembrolizumab (KEYTRUDA® (pembrolizumab) [package insert]. Merck & Co., Inc., Whitehouse Station, NJ; 2019). Participants enrolled in this study will be monitored for signs and symptoms of infusion-related reactions including rigors, chills, wheezing, pruritus, flushing, rash, hypotension, hypoxemia, and fever.

The primary objective in this study is to assess the pharmacodynamic effects of pembrolizumab administered by the SOFUSA® DoseConnect™ device (DoseConnect) in participants with relapsed or refractory cutaneous T-cell lymphoma (R/R CTCL). Endpoints include T-cell exhaustion markers (e.g., PD-1, Lag-3, Tim-3, and ICOS in malignant CD4+ and tumor-infiltrating CD8 T-cells in tumor tissue); detection of pembrolizumab in tumor tissue; and Ki67 expression in tumor tissue.

Secondary objectives include (1) to assess the safety of pembrolizumab administered by the DoseConnect in participants with R/R CTCL, with endpoints including types, frequencies, and severities of adverse events (AEs) and the relationships of AEs to study intervention; includes serious adverse events (SAEs); and (2) to assess the PK of pembrolizumab administered by the DoseConnect in participants with R/R CTCL, with endpoints including PK parameters Cmax, Tmax, AUC, and t½ of pembrolizumab.

Exploratory objectives include (1) to assess the activity of pembrolizumab administered via SOFUSA® DoseConnect™ in participants with R/R CTCL, with endpoints including Objective Response Rate (ORR) as assessed by the Investigator per Global Response Score (GRS) (Olsen E A, Whittaker S, Kim Y H, et al. Clinical end points and response criteria in mycosis fungoides and Sézary syndrome: a consensus statement of the International Society for Cutaneous Lymphomas, the United States Cutaneous Lymphoma Consortium, and the Cutaneous Lymphoma Task Force of the European Organisation for Research and Treatment of Cancer. J Clin Oncol. 2011; 29(18):2598-2607, incorporated herein by reference); Duration of Response (DOR) as assessed by Investigator per the GRS; ORR in the skin based on Modified Severity Weighted Assessment Tool (mSWAT) for response in skin; ORR in the skin based on the Composite Assessment of Index Lesion Severity (CAILS) score; and Reduction in peripheral Sezary count in those participants with detectable Sezary count at baseline; (2) To assess additional pharmacodynamic effects of pembrolizumab administered by the DoseConnect in participants with CTCL, with endpoints including Ki67 expression in blood; receptor occupancy of pembrolizumab in blood; and analysis of lymphatic flow in relation to response, safety, PK; (3) To evaluate any pain associated with use of the DoseConnect, with endpoints including assessment of pain with use of DoseConnect using the Visual Analog Scale (VAS); and (4) To evaluate skin irritation associated with use of the DoseConnect, with endpoints including assessment of skin irritation at DoseConnect application site using a Modified Draize Scale.

The overall design will be an open-label, single-center pilot study to investigate the pharmacodynamics, pharmacokinetics (PK), safety, and activity of pembrolizumab administered intra-lymphatically using the DoseConnect in participants with relapsed or refractory cutaneous T-cell lymphoma (CTCL). All participants will receive the study intervention of pembrolizumab administered intra-lymphatically using the SOFUSA® DoseConnect™. The study will consist of a Screening Period, a Treatment Period and an Extended Treatment Period. The Screening Period for eligibility determination begins upon a participant's written informed consent. All screening assessments must be completed within 28 days prior to start of Cycle 1. The Treatment Period begins with the first dose of study intervention. Each cycle will be 21 days/3 weeks. Eligible participants will receive the pembrolizumab administered intra-lymphatically using the DoseConnect every week (Q1W) for the first 2 cycles, and then, per Investigator discretion, either continue the pembrolizumab Q1W dosing or switch to pembrolizumab every 3 weeks (Q3W) dosing starting at Cycle 3. Participants who complete 8 cycles of treatment may elect either of the following: a. discontinue study and receive standard of care treatment which may include an anti-PD-1 antibody agent given intravenously or b. upon agreement with the sponsor, enter the Extended Treatment Period and continue to receive the study intervention, pembrolizumab Q1W or pembrolizumab Q3W administered via the DoseConnect until PD, unacceptable toxicity, death, lost to follow-up, withdrawal of consent or termination of the study by the Sponsor.

All participants will return to the site for an End of Study (EOS) visit at 28 days (+3 days) after the last dose of study intervention.

Skin punch/core needle biopsy will be performed at Cycle 1 Day 1 predose and Cycle 2 Day 1 postdose of: 1) target CTCL lesion (skin or lymph node [LN]) that is proximal to the intended DoseConnect placement and 2) if present, lesion located either distal to intended device placement or on an extremity that is opposite of the respective arm/leg intended for device placement. The preferred DoseConnect placement is on the upper or lower extremities, except the thighs. At these biopsy timepoints, it is preferred the DoseConnect be placed on the same location and the same two lesions are biopsied. However, if the DoseConnect must be placed on a different location at the post baseline timepoint, then biopsies should be performed based on the location of the device at that timepoint (one lesion downstream of the lymphatic flow from the DoseConnect placement and, if present, one lesion which is non-downstream).

Prior to the study intervention dosing, lymphatic imaging using indocyanine green (ICG) solution administered via the DoseConnect will be performed and recorded. The lymphatic imaging is being performed to determine the lymphatic pumping rates to and from the targeted tumor lesion that will biopsied.

This study will use commercially available pembrolizumab (Keytruda®) (Merck).

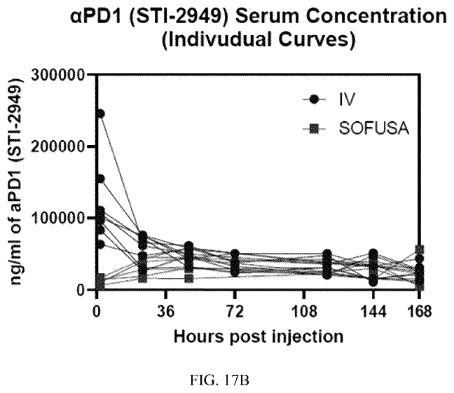

Dosing of pembrolizumab using the DoseConnect will be as shown in FIG. 16.

Key efficacy assessments will include (1) Modified Severity Weighted Assessment Tool (mSWAT) for response in skin, (2) Composite Assessment of Index Lesion Severity for response in skin, (3) Flow cytometry for Sezary cell count for response in blood, (4) PET/CT scan for participants with stage IB disease with >30% skin involvement, stage IIB-IVB disease, Sezary Syndrome (SS), or transformed Mycosis Fungoides (MF). Scans not needed for stage IB participants or participants with <30% skin involvement, and (5) Global Response Score for assessment of response.

Key safety assessments will include (1) Adverse Events attributable to drug, device or both, (2) Clinical laboratory assessments, (3) Physical Examination, (4) Vital signs, (5) Electrocardiogram (ECG), (6) Ophthalmologic exam (If clinically indicated due to signs or symptoms of uveitis), (7) Monitoring for infusion-related reactions, and (8) Assessment of pain/skin irritation due to DoseConnect.

Key pharmacokinetic assessment will include blood sampling for PK parameters.

Key pharmacodynamic assessments will include (1) Blood and tumor tissue sampling for pharmacodynamic parameters, and (2) Lymphatic imaging with the ICG solution using DoseConnect to assess lymphatic flow.

In this study, pembrolizumab will be administered via the DoseConnect in patient with CTCL to assess through pharmacodynamic assessment in the tumor tissue if lymphatic delivery of pembrolizumab is feasible. The choice of CTCL is based on accessibility of tumor cells for pharmacodynamic measurements.

The planned sample size is 10 participants. For safety, for the first 5 participants, the enrollment rate will be one participant every three weeks or longer. The additional 5 participants will only be enrolled if the DRC determines it is acceptable based on review of data from the initial 5 participants. If at any time during study conduct. a ≥Grade 3 AE attributable to either drug or device or ≥Grade 2 AE lasting ≥1 week and attributable to device is reported, the enrollment will be halted, and the DRC will be convened to review the data and determine the course of the study conduct.

This clinical trial is a pilot study intended to assess the feasibility of drug administration via SOFUSA® and yield preliminary data on the biological effects of lymphatic administration of an anti-PD-1 agent. The primary endpoint of biomarker assessment has been chosen to provide preliminary evidence of biological activity when an anti-PD-1 agent is given via the DoseConnect. The biomarkers chosen, T-cell exhaustion markers PD-1, Lag-3, Tim-3, and ICOS in malignant CD4+ and tumor-infiltrating CD8 T-cells in tumor tissue, have been previously studied in many malignancies including CTCL (Querfeld C, Zain J M, Wakefield D L, et al. Phase 1/2 Trial of Durvalumab and Lenalidomide in Patients with Cutaneous T Cell Lymphoma (CTCL): Preliminary Results of Phase I Results and Correlative Studies, Blood 2018a 132:2931; Querfeld C (2018b, December). Phase 1/2 Trial of Durvalumab and Lenalidomide in Patients with Cutaneous T Cell Lymphoma (CTCL): Preliminary Results of Phase I Results and Correlative Studies. Oral presentation at the annual meeting of the American Society of Hematology. San Diego, CA). In addition, detection of pembrolizumab in tumor tissue is based on previous observations in preclinical models that SOFUSA® administration can lead to higher levels of drug in the lymph nodes and tumor tissue compared to intravenous or other systemic administration methods. Ki67 in the tumor bed and peripheral blood can be detected after anti-PD-1 therapy (Kamphorst A O, Pillai R N, Yang S, Nasti T H, Akondy R S, Wieland A, Sica G L, Yu K, Koenig L, Patel N T, Behera M, Wu H, McCausland M, Chen Z, Zhang C, Khuri F R, Owonikoko T K, Ahmed R, Ramalingam S S. Proliferation of PD-1+CD8 T cells in peripheral blood after PD-1-targeted therapy in lung cancer patients. Proc Natl Acad Sci USA. 2017 May 9; 114(19):4993-4998) and is chosen as one measure of systemic and possibly abscopal effects of pembrolizumab treatment using the DoseConnect.

Although the delivery of pembrolizumab will be via the lymphatics using the DoseConnect, since the lymphatic system is connected to the venous system, it is expected the intra-lymphatically administered drug will eventually appear in the blood and therefore allow for pharmacokinetic and pharmacodynamic measurements in the blood.

### Example 7. SOFUSA® DoseConnect™ Delivery of Anti-PD-1 (STI-2949) Antibody Results in 69% Bioavailability Compared to I.V. Administration in Sprague Dawley Rats

Sprague Dawley rats weighing ˜165 grams were administered with a single dose of 1 mg anti-PD1 monoclonal antibody STI-2949 (Sorrento Pharmaceuticals; also referred to herein as STI-A1110) intravenously (i.v.) or using SOFUSA® DoseConnect™.

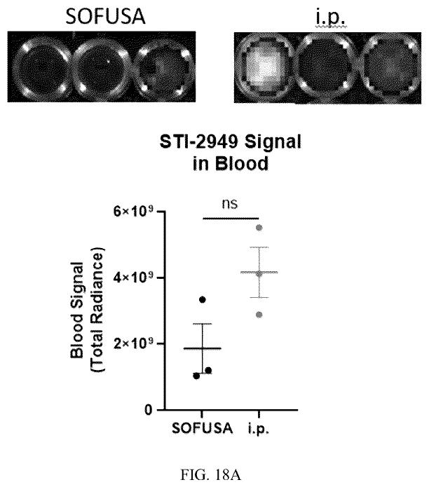

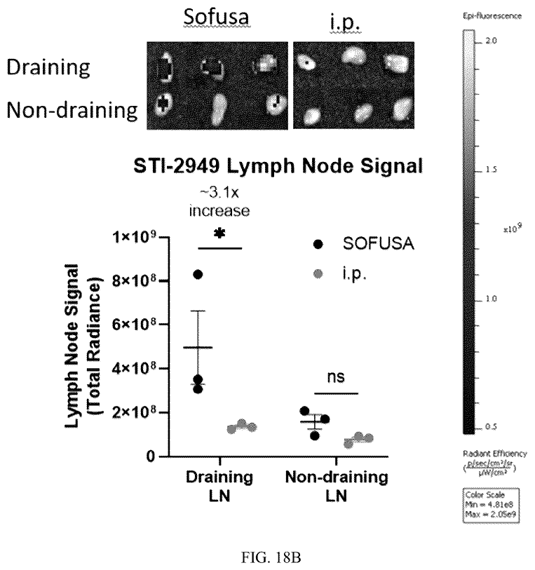

Following administration, serum was collected at the time points indicated in FIG. 17A for 7 days.

Anti-PD1 concentration in serum was determined using an ELISA with PD1 coated plates all at the same serum dilutions.

As shown in FIG. 17A, following SOFUSA® DoseConnect™ administration, average anti-PD1 serum bioavailability was 69.09% over the time period assessed. FIG. 17B shows the data for all individual animals.

### Example 8. SOFUSA® DoseConnect™ Delivery of Fluorescently Tagged Anti-PD-1 (STI-2949) Antibody Leads to Lower Blood Concentrations while Increasing Draining Lymph Node Concentrations 24 Hours Post Injection

Anti-PD1 antibody STI-2949 was fluorescently labeled with AlexaFluor 647 and mice were administered intraperitoneally (i.p.) and via SOFUSA® DoseConnect™. Twenty-four hours later, blood, draining lymph nodes and non-draining lymph nodes were taken from the mice. Radiance levels of the fluorescently labeled anti-PD1 antibodies were assessed in the blood, draining lymph nodes and non-draining lymph nodes.

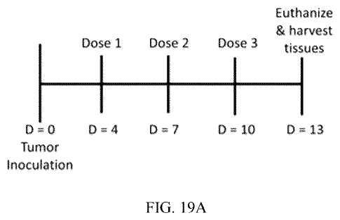

As shown in FIG. 18A, SOFUSA® DoseConnect™ delivery of fluorescently tagged anti-PD1 (STI-2949) antibody leads to approximately 2-fold lower blood concentrations compared to i.p. administration. Furthermore, as shown in FIG. 18A, SOFUSA® DoseConnect™ delivery of fluorescently tagged anti-PD1 (STI-2949) antibody leads to approximately 3-fold increased draining lymph node concentrations compared to i.p. administration.

### Example 9. SOFUSA® DoseConnect™ Mediated Delivery of Anti-PD-1 Antibody Towards Tumor-Draining Lymph Nodes Allows Dose Reductions in MC38 Colon Carcinoma Model

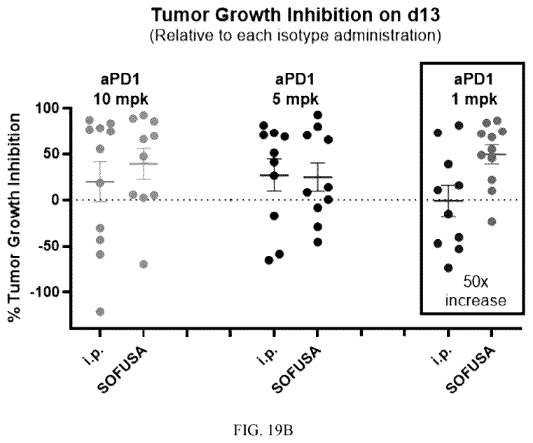

As shown schematically in FIG. 19A, mice were inoculated with MC38 tumor cells and thereafter administered at 4, 7, and 10 days post-implant with monoclonal anti-PD1 (BB9 clone, Sorrento Pharmaceuticals) at 10 mg/kg, 5 mg/kg, or 1 mg/kg, either intraperitoneally or via SOFUSA® DoseConnect™. The anti-PD1 BB9 clone is described in U.S. Provisional Application 63/044,808 filed Jun. 26, 2020, the disclosure of which is incorporated herein by reference in its entirety.

Mice were euthanized and tumor tissue harvested at 13 days post-implant.

As shown in FIG. 19B, efficacy of tumor growth inhibition was similar when mice were administered with anti-PD1 BB9 at 10 mg/kg or 5 mg/kg, either intraperitoneally or via SOFUSA® DoseConnect™. However, SOFUSA® DoseConnect™ administration of anti-PD1 at the lowest dose tested of 1 mg/kg increased tumor growth inhibition by approximately 50-fold compared to the same dose administered intraperitoneally. In the graph shown in FIG. 19B, higher tumor growth inhibition percentage results in smaller tumors.

Aspects of these various examples may all be combined with one another, even if not expressly combined in the present disclosure, unless they are clearly mutually exclusive. For example, a specific pharmaceutical formulation may contain amounts of components identified more generally or may be administered in any way described herein.

In addition, various example materials are discussed herein and are identified as examples, as suitable materials, and as materials included within a more generally-described type of material, for example by use of the term “including” or “such-as.” All such terms are used without limitation, such that other materials falling within the same general type exemplified but not expressly identified may be used in the present disclosure as well.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

Various patents, patent applications and publications are cited herein, the contents of which are hereby incorporated in their entireties.

1. A method of treating cancer in a patient, comprising:
placing a device comprising a plurality of microneedles on the skin of the patient proximate to a first position under the skin of the patient, wherein the first position is proximate to lymph vessels and/or lymph capillaries in the patient's lymphatic system, and wherein the microneedles have a surface comprising nanotopography;
inserting the plurality of microneedles into the patient to a depth whereby at least the epidermis is penetrated and an end of at least one of the microneedles is proximate to the first position; and
administering via the plurality of microneedles to the first position an effective amount of an anti-PD-1 therapeutic agent or an effective amount of an anti-PD-L1 therapeutic agent to treat cancer in a patient.

2. A method of preventing or reducing cancer metastasis in a patient, comprising:
locating at least one lymph node in the patient that intervenes in the lymphatic system between a solid cancer tumor and a draining duct;
placing a device comprising a plurality of microneedles on the skin of the patient proximate to a first position under the skin of the patient located between the intervening lymph node and the solid cancer tumor, wherein the first position is proximate to lymph vessels and/or lymph capillaries in the patient's lymphatic system, and wherein the microneedles have a surface comprising nanotopography;
inserting the plurality of microneedles into the patient to a depth whereby at least the epidermis is penetrated and an end of at least one of the microneedles is proximate to the first position; and
administering via the plurality of microneedles to the first position a therapeutically effective amount of an anti-PD-1 therapeutic agent or an anti-PD-L1 therapeutic agent that is effective for preventing or reducing cancer metastasis in the patient.

3. A method of preventing or reducing cancer metastasis in a patient, comprising:
locating a solid cancer tumor in the patient;
locating at least one lymph node in the patient that intervenes in the lymphatic system between the solid cancer tumor and a draining duct;
placing a device that comprises a plurality of microneedles on the skin of the patient at a first location on the skin of the patient that is proximate to lymph capillaries and/or lymph vessels that flow into the intervening lymph node, wherein the microneedles have a surface comprising nanotopography;
inserting the plurality of microneedles into the patient to a depth whereby at least the epidermis is penetrated; and
administering via the plurality of microneedles to the lymph capillaries and/or lymph vessels that flow into the intervening lymph node a therapeutically effective amount of an anti-PD-1 therapeutic agent or an anti-PD-L1 therapeutic agent that is effective in preventing or reducing cancer metastasis in the patient.

4. The method of claim 1, wherein the cancer comprises a tumor.

5. The method of claim 1, wherein the lymph node is a tumor draining lymph node.

6. The method of claim 1, wherein the cancer is a cancer susceptible to treatment with an anti-PD-1 therapeutic agent or anti-PD-L1 therapeutic agent.

7. The method of claim 1, wherein serum bioavailability of the anti-PD-1 therapeutic agent or the anti-PD-L1 therapeutic agent is up to 10%, 15% 20%, 25%, 30%, 35%, 40%, 45%, 50%, 55%, 60%, 65%, or 70%.

8. The method of claim 1, wherein a serum Tmax of the anti-PD-1 therapeutic agent or the anti-PD-L1 therapeutic agent is from 10 to 100 hours.

9. The method of claim 1, wherein a serum Cmax of the anti-PD-1 therapeutic agent or the anti-PD-L1 therapeutic agent is decreased up to 1.5-fold, 2-fold, 2.5-fold, 3-fold, 3.5-fold, or 4-fold, as compared to a serum Cmax following an equivalent amount of the anti-PD-1 therapeutic agent or the anti-PD-L1 therapeutic agent administered by an intravenous delivery route.

10. The method of claim 1, wherein a serum AUC₀₋ₜ of the anti-PD-1 therapeutic agent or the anti-PD-L1 therapeutic agent is decreased up to 1.5-fold, 2-fold, 2.5-fold, 3-fold, 3.5-fold, or 4-fold as compared to a serum AUC₀₋ₜ following an equivalent amount of the anti-PD-1 therapeutic agent or the anti-PD-L1 therapeutic agent administered by an intravenous delivery route.

11. The method of claim 1, wherein delivery of the anti-PD-1 therapeutic agent or the anti-PD-L1 therapeutic agent to one or more lymph nodes is increased up to 1.5-fold, 2-fold, 2.5-fold, 3-fold, 3.5-fold, or 4-fold as compared to delivery to one or more lymph nodes of an equivalent amount of the anti-PD-1 therapeutic agent or the anti-PD-L1 therapeutic agent administered by an intravenous delivery route.

12. The method of claim 1, wherein levels of the anti-PD-1 therapeutic agent or anti-PD-L1 therapeutic agent in one or more systemic organs is decreased 10-75% over a period of time as compared to levels in one or more systemic organs following an equivalent amount of the anti-PD-1 therapeutic agent or the anti-PD-L1 therapeutic agent administered by an intravenous delivery route over a same period of time.

13. The method of claim 12, wherein the organ is a liver or a kidney.

14. The method of claim 12, wherein the period of time is up to 12 hours, 24 hours, 36 hours, 48 hours, 60 hours, or 72 hours.

15. The method of claim 1, wherein the anti-PD-1 therapeutic agent or the anti-PD-L1 therapeutic agent is at least 90%, 95%, 99%, or 99.9% cleared from the patient's serum by 28 days after administration.

16. The method of claim 1, wherein the administering results in an undetectable primary tumor and/or an undetectable secondary tumor.

17. The method of claim 16, wherein an incidence or probability of an undetectable primary tumor and/or an undetectable secondary tumor is at least 30%, 40%, 50%, 60%, 70%, 80%, 90%, 95%, or 99%.

18. The method of claim 1, wherein exposure of the anti-PD-1 therapeutic agent or the anti-PD-L1 therapeutic agent to T-cells in the patient's lymphatic system is increased up to 1.5-fold, 2-fold, or 2.5-fold, as compared to exposure to T-cells in the patient's lymphatic system following an equivalent amount of the anti-PD-1 therapeutic agent or the anti-PD-L1 therapeutic agent administered by an intravenous delivery route.

19. The method of claim 1, wherein exposure of the anti-PD-1 therapeutic agent or the anti-PD-L1 therapeutic agent to one or more solid cancer tumors in the patient's lymphatic system is increased up to 1.5-fold, 2-fold, or 2.5-fold, as compared to exposure to one or more solid cancer tumors in the patient's lymphatic system following an equivalent amount of the anti-PD-1 therapeutic agent or the anti-PD-L1 therapeutic agent administered by an intravenous delivery route.

20. The method of claim 1, wherein tumor-infiltrating lymphocytes are increased up to 1.5-fold, 2-fold, or 2.5-fold as compared to tumor-infiltrating lymphocytes following an equivalent amount of the anti-PD-1 therapeutic agent or the anti-PD-L1 therapeutic agent administered by an intravenous delivery route.

21. The method of claim 1, wherein incidence or severity of one or more immune-related Adverse Events is reduced as compared to one or more immune-related Adverse Events following an equivalent amount of the anti-PD-1 therapeutic agent or the anti-PD-L1 therapeutic agent administered by an intravenous delivery route.

22. The method of claim 1, wherein tumor growth inhibition is increased up to 10-fold, 20-fold, 30-fold, 40-fold, or 50-fold compared to tumor growth inhibition following an equivalent amount of the anti-PD-1 therapeutic agent or the anti-PD-L1 therapeutic agent administered by an intravenous delivery route.

23. The method of claim 1, wherein tumor growth inhibition is equal to or better than tumor growth inhibition following up to 1.5-fold, 2-fold, 2.5-fold, 3-fold, 3.5-fold, 4-fold, 4.5-fold, or 5-fold greater amount of the anti-PD-1 therapeutic agent or the anti-PD-L1 therapeutic agent administered by an intravenous delivery route.
